(19) United States
(12) Patent Application Publication
(10) Pub. No.: US 2026/0225906 A1
(43) Pub. Date: Aug. 6, 2026

(54) METHOD FOR CONVERTING CARBON DIOXIDE INTO CARBONATE NANOMATERIALS USING BIOLOGICAL MATERIALS

(71) Applicant: West Virginia University Board of Governors on Behalf of West Virginia University, Morgantown, WV (US)

(72) Inventors: Bingyun LI, Morgantown, WV (US); Qingyang LI, Morgantown, WV (US); Kñeisha McDonald, Morgantown, WV (US)

(21) Appl. No.: 19/466,600

(22) Filed: Feb. 2, 2026

Related U.S. Application Data
(60) Provisional application No. 63/754,686, filed on Feb. 6, 2025.

(51) Int. Cl. C01F 11/18; A61L 15/18; C01G 5/00; H01F 27/32; H01G 4/12

(52) U.S. Cl. CPC C01F 11/183; A61L 15/18; C01F 11/187; C01F 11/188; C01G 5/00; H01F 27/32; H01G 4/1209; C01P 2002/72; C01P 2002/86; C01P 2004/03; C01P 2004/16; C01P 2004/62; C01P 2004/64

(57) ABSTRACT

In one aspect, the disclosure relates to metal carbonate nanoparticles, methods of making the same, and articles and compositions using the same. In an aspect the method can be a two-step method comprising bubbling a gas mixture containing carbon dioxide through a solution of a base and an amino acid, followed by addition of a metal salt and subsequent precipitation of the metal carbonate nanoparticles. In an alternative aspect, the method can be a one-step method comprising bubbling a gas mixture containing carbon dioxide through a solution of a base and an amino acid. In either of these methods, the amino acids can be recovered and re-used. In one aspect, the morphology and size of the nanoparticles can be tailored based on amino acid concentration and identity. In any of these aspects, the method is further useful for capturing and/or sequestering carbon dioxide from flue gases or the atmosphere.

### CROSS-REFERENCE TO RELATED APPLICATIONS

This application claims the benefit of U.S. Provisional Application No. 63/754,686 filed on Feb. 6, 2025, which is incorporated herein by reference in its entirety.

### STATEMENT REGARDING FEDERALLY SPONSORED RESEARCH OR DEVELOPMENT

This invention was made with government support under grant number FE0031707 awarded by the United States Department of Energy, grant number NIFA 2020-68012-31881 awarded by the United States Department of Agriculture, and R01Al177620 awarded by the National Institutes of Health. The government has certain rights in the invention.

### BACKGROUND

Atmospheric CO₂ levels are now at their highest in millions of years, driving a host of catastrophic effects, including extreme weather events, rising sea levels, ocean acidification, disrupted ecosystems, deteriorating air quality, respiratory health issues, heat-related illnesses, threats to food and water security, and escalating social and geopolitical tensions. Development of methods that effectively reduce CO₂ emission from power plants and other sources through carbon capture technologies is urgently needed. Ideally, such methods would convert the captured CO₂ into commercially valuable products and/or stable products that can be stored as needed.

Meanwhile, nanomaterials have many unique properties and promising applications including uses such as fillers, medicines, and catalysts, and in environmental, biomedical, and electronic applications, and in industries ranging from food and construction to paint and polymers. However, methods to produce nanomaterials in large quantities and at low cost are challenging. Similarly, methods that can produce various carbonate products at scale are presently lacking. Carbonate nanomaterials represent an area of particular interest. It is expected that specific morphologies of nanomaterials may be important for specific applications; thus, the ability to tailor the morphology of carbonate nanomaterials would be particularly useful. An ideal method to produce carbonate nanomaterials would take advantage of the thermodynamic favorability of their formation from various precursors.

Despite advances in carbon capture and storage research, as well as nanomaterials research, there is still a scarcity of low-cost, scalable methods for producing carbonate nanomaterials from atmospheric CO₂ and/or flue gases containing CO₂. Ideally the nanomaterials would exhibit customizable morphologies and could be tailored to specific applications. These needs and other needs are satisfied by the present disclosure.

### SUMMARY

In accordance with the purpose(s) of the present disclosure, as embodied and broadly described herein, the disclosure, in one aspect, relates to metal carbonate nanoparticles, methods of making the same, and articles and compositions using the same. In an aspect the method can be a two-step method comprising bubbling a gas mixture containing carbon dioxide through a solution of a base and an amino acid or other biological compounds, including amines, followed by addition of a metal salt and subsequent precipitation of the metal carbonate nanoparticles. In an alternative aspect, the method can be a one-step method. In either of these methods, the amino acids can be recovered and re-used. In one aspect, the morphology and size of the nanoparticles can be tailored based on amino acid concentration and identity as well as other factors. In any of these aspects, the method is further useful for capturing and/or sequestering carbon dioxide from flue gases and the atmosphere.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a two-step scheme to convert CO₂ into carbonate nanomaterials.

FIGS. 2A-2D show (FIG. 2A) an innovative two-step process converting CO₂ into CaCO₃ nanoparticles. (FIG. 2B) NMR spectra of solvent and the precipitates after the two-step process. (FIG. 2C) XRD spectra of standard calcite and the nanoparticles produced. (FIG. 2D) Size distribution from SEM images of the CaCO₃ nanoparticles produced (480 particles in 4 images). Experimental conditions: glycine (Gly) (0.05 mol), NaOH (0.05 mol), CaCl₂) (0.05 mol), CO₂ gas flow rate (100 sccm), and CO₂ bubbling time (20 min).

FIGS. 3A-3D show (FIG. 3A) precipitates produced using different doses of Gly (0 mol, 0.025 mol, and 0.05 mol). (FIG. 3B) Schematic diagram describing the evolution with Gly dosed of micro- and nanoparticle formation. (FIG. 3C) Percentage of nanoparticles produced at different doses of Gly and carbamate amount at 20 min at different doses of Gly before adding CaCl₂). (FIG. 3D) CO₂ loading after bubbling CO₂ for 20 min. Experimental conditions: Gly (0-0.10 mol), NaOH (0.05 mol), CaCl₂) (0.05 mol), CO₂ gas flow rate (100 sccm), and CO₂ bubbling time (20 min).

FIGS. 4A-4D show CaCO₃ nanoparticles prepared from (FIG. 4A) asparagine (Asn), (FIG. 4B) alanine (Ala), and (FIG. 4C) cysteine (Cys). (FIG. 4D) XRD spectra of the nanoparticles prepared from various amino acids. Experimental conditions: Asn (0.05 mol), Ala (0.05 mol), Cys (0.05 mol), NaOH (0.05 mol), CaCl₂) (0.05 mol), CO₂ gas flow rate (100 sccm), and CO₂ bubbling time (20 min).

FIGS. 5A-5F show SEM images of precipitates produced (FIG. 5A) without and (FIG. 5B) with Gly. NMR spectra (FIG. 5C) without and (FIG. 5D) with Gly. (FIG. 5E) Gly and carbamate percentage at different CO₂ bubbling time with Gly. (FIG. 5F) XRD spectra without and with Gly. In the spectra in FIGS. 5C-5D, peaks 1 and 2 represent Gly/Gly⁻, peaks 3, 4, and 5 represent carbamate, and peak 6 represents CO₃²⁻/HCO₃⁻. 1,4-dioxane was used as the control reference (ref). Experimental conditions: Gly (0 or 0.05 mol), NaOH (0.05 mol), CaCl₂) (0.05 mol), CO₂ gas flow rate (100 sccm), and CO₂ bubbling time (20 min).

FIGS. 6A-6D show adding BaCl₂ to mineralize CO₂ in glycinate solvent (Gly+NaOH). (FIG. 6A) changes of CO₂ loading and pH vs CO₂ bubbling time before adding BaCl₂. (FIG. 6B) SEM image of the precipitates after BaCl₂ was added at 50 min. (FIG. 6C) XRD spectra of precipitates. (FIG. 6D) NMR spectra of the solvent at 0, 5, 10, 30, and 50 min of CO₂ bubbling before adding BaCl₂, and after adding BaCl₂ at 50 min. Experimental conditions: Gly (0.025 mol), NaOH (0.025 mol), BaCl₂ (0.0125 mol), CO₂ gas flow rate (50 sccm), and bubbling time (50 min).

FIGS. 7A-7C show SEM images of the precipitates where (FIG. 7A) Ser and (FIGS. 7B-7C) Lys were used. Experimental conditions: Ser or Lys (0.025 mol), NaOH (0.025 mol), BaCl₂ (0.0125 mol), CO₂ gas flow rate (50 sccm), and bubbling time (50 min).

FIGS. 8A-8C show two steps used to convert CO₂ into Ag₂CO₃ nanoparticles: (FIG. 8A) TEM image, (FIG. 8B) SEM image, and (FIG. 8C) XRD spectra of precipitates. Experimental conditions: Gly (0.0125 mol), NaOH (0.0125 mol), AgNO₃ (0.0125 mol), CO₂ gas flow rate (50 sccm), and CO₂ bubbling time (15 min).

FIG. 9 shows a one-step process to convert CO₂ into carbonate nanomaterials. An aqueous solution of an amino acid and a base like Ba(OH)₂ is bubbled with a CO₂-containing gas to form BaCO₃ precipitates in one-step. After removing the precipitates, the remaining solvent containing the amino acid is obtained and can be re-used while the base needs to be re-plenished.

FIGS. 10A-10F show BaCO₃ formed from five cycles by re-using Gly and Ba(OH)₂ for CO₂ absorption. (FIGS. 10A-10E) SEM images of precipitates produced in the 1st, 2nd, 3rd, 4th, and 5th cycles, respectively. (FIG. 10F) Conversion ratio of Ba²⁺ to nano-BaCO₃ over the five consecutive cycles. Experimental conditions: Gly (0.025 mol), Ba(OH)₂ (0.0125 mol), CO₂ gas flow rate (50 sccm), and CO₂ bubbling time (50 min).

FIGS. 11A-11D show capturing and converting CO₂ to BaCO₃ using different amino acids. SEM image of the precipitates using (FIG. 11A) Cys, (FIG. 11B) Ser, and (FIG. 11C) Lys. (FIG. 11D) XRD spectra of precipitates using different amino acids. Experimental conditions: Ba(OH)₂ (0.0125 mol), CO₂ gas flow rate (50 sccm), and bubbling time (50 min).

FIGS. 12A-12D show absorption and conversion of CO₂ to CaCO₃ nanomaterials: (FIG. 12A) Phase separation after 24-hour. (FIG. 12B) NMR spectra of upper solution and precipitates after phase separation. (FIG. 12C) SEM image and TEM image (inset) of precipitates. (FIG. 12D) XRD spectra of standard calcite and nanoparticle precipitates.

FIGS. 13A-13C show CaCO₃ formation when bubbling SFG of varying CO₂ concentrations (i.e., 4%, 12%, and 20%). FIG. 13A) Change of CO₂ absorption vs. flue gas bubbling time. FIG. 13B) Carbamate percentage from NMR data before CaCl₂) addition. FIG. 13C shows SEM images of nano-CaCO₃ produced from simulated flue gas (SFG) of different CO₂ concentration (i.e., 4%, 12%, and 20%). Experimental conditions: Gly (0.025 mol), NaOH (0.025 mol), and CaCl₂) (0.0125 mol).

FIGS. 14A-14B show CO₂ capture and conversion using real flue gas (RFG) as the CO₂ source. (FIG. 14A) XRD spectra of calcite and vaterite from the American Mineralogist Crystal Structure Database, and precipitates from SFG and RFG. (FIG. 14B) SEM (left) and TEM (right) images of the nanoparticles formed. Experimental conditions: Gly (0.025 mol), NaOH (0.025 mol), CaCl₂) (0.0125 mol), and gas flow rate (200 sccm).

FIGS. 15A-15D show a comparison of two solvents with and without Gly for CO₂ capture and mineralization. FIG. 15A) CO₂ absorption curve prior to the addition of CaCl₂). FIG. 15B) XRD spectra of precipitates after CaCl₂) was added. FIG. 15C) SEM images of precipitates produced. FIG. 15D) NMR spectra before and after the addition of CaCl₂) at 30 min. Each experiment was conducted with three replicates.

FIGS. 16A-16C show CaCO₃ formation when CaCl₂) was added at different SFG bubbling time points. FIG. 16A) Variations of CO₂ loading and carbamate amount over SFG bubbling time. FIG. 16B) Amount, and FIG. 16C) SEM images of CaCO₃ generated when CaCl₂) was added at different time points. p-value: ***<0.001. Each experiment was conducted with three replicates.

FIGS. 17A-17C show CaCO₃ formation using SFG with different CO₂ concentrations (4%, 12%, 20%). FIG. 17A) Change of CO₂ absorption vs. SFG bubbling time. FIG. 17B) Carbamate percentage from NMR data at 30 min before CaCl₂) addition. FIG. 17C) SEM images of CaCO₃ produced. p-value: ***<0.001. Each experiment was conducted with three replicates.

FIGS. 18A-18C show CaCO₃ formation when CO₂ was fixed with different N₂ flow rates to form flue gas. FIG. 18A) Change of CO₂ absorption vs. SFG bubbling time. FIG. 18B) Carbamate percentage from NMR data at 30 min before CaCl₂) addition. FIG. 18C) SEM images of CaCO₃ precipitates produced. Each experiment was conducted with three replicates.

FIGS. 19A-19B show cell viability upon exposure to CaCO₃ nanoparticles produced by FIG. 19A) RFG and FIG. 19B) SFG under different time and concentration. p-values: *p<0.05, **p<0.01, ***p<0.001, ****p<0.0001. All results were performed in duplicate with three replicates each. Data were expressed as the means±standard deviation.

FIGS. 20A-20C show a comparison of cell viability upon exposure to CaCO₃ nanoparticles produced by RFG and SFG with the concentration of FIG. 20A) 10 μg/mL, FIG. 20B) 100 μg/mL, and FIG. 20C) 400 μg/mL under different time. p-values: *p<0.05, **p<0.001, ***p<0.001, ****p<0.0001. All results were performed in duplicates with three replicates each. Data were expressed as means±standard deviation.

FIGS. 21A-21B show (FIG. 21A) CO₂ loading and pH change as a function of flue gas bubbling time. (FIG. 21B) NMR spectra of the amino acid salt solvent before and during flue gas bubbling. Experimental conditions: NaOH (0.025 mol), Gly (0.075 mol), flue gas (12% CO₂), bubbling time (30 min), and temperature (60° C.).

FIG. 22A shows NMR spectra of (top) solvent and (bottom) sample from washing the precipitates. FIG. 22B shows a TEM image of washed precipitates. Experimental conditions: NaOH (0.025 mol), Gly (0.075 mol), flue gas (12% CO₂), CaO (0.009 mol), bubbling time (15 min), and temperature (60° C.).

FIG. 23A shows relative concentration of Gly. FIG. 23B shows CO₂ loading. Experimental conditions: NaOH (0.025 mol), Gly (0, 0.025, 0.050, 0.075 mol), flue gas (12% CO₂), bubbling time (15 min), and temperature (60° C.). NS: not statistically significant.

FIGS. 24A-24C show (FIG. 24A) yields, (FIG. 24B) SEM images, and (FIG. 24C) XRD spectra of formed CaCO₃ using different Gly doses. Experimental conditions: NaOH (0.025 mol), Gly (0, 0.025, 0.050, and 0.075 mol), CaO (0.009 mol), flue gas (12% CO₂), bubbling time (15 min), and temperature (60° C.). p-value: *<0.05; **<0.01; ***<0.001.

FIGS. 25A-25B show solvent change during cycling. (FIG. 25A) The Gly amount changes during cycling. (FIG. 25B) The pH changes during cycling. Experimental conditions: NaOH (0.025 mol), Gly (0.075 mol), flue gas (12% CO₂), bubbling time (15 min), and temperature (60° C.).

FIGS. 26A-26B show product observation during cycling. (FIG. 26A) SEM images of CaCO₃ after each cycle. (FIG. 26B) CaO conversion rate of each cycle. Experimental conditions: NaOH (0.025 mol), Gly (0.075 mol), CaO (0.009 mol), flue gas (12% CO₂), bubbling time (15 min), and temperature (60° C.). p-value: ****<0.0001.

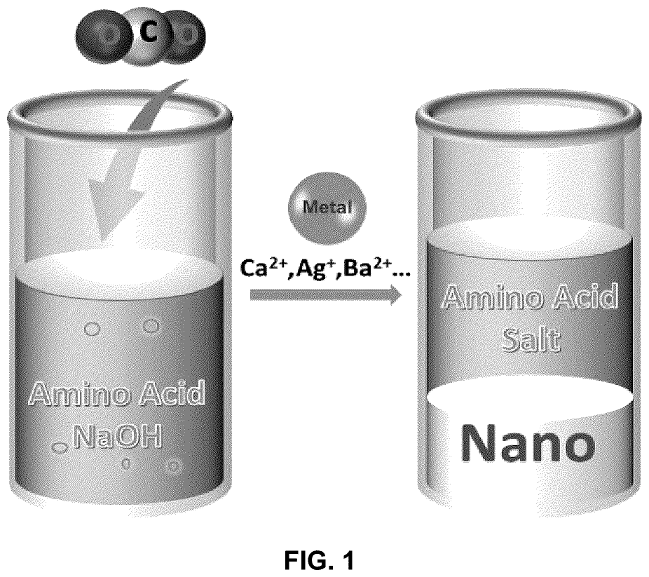

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The utilization and management of carbon dioxide (CO₂) have emerged as critical areas of focus for scientists worldwide. While CO₂ is prevalent in various industrial gas streams—typically ranging from 4-20 vol % in industrial flue gases—these sources collectively represent a significant and largely untapped carbon resource. In one aspect, a primary challenge in effectively harnessing this resource for beneficial applications, such as the production of chemicals, foods, and advanced materials, stems from the energy-intensive processes currently required for its efficient capture and purification. In another aspect, advancements in these areas are important for the broader implementation of CO₂ conversion technologies. In an aspect, flue gas primarily consists of nitrogen (N₂) and CO₂, along with trace amounts of particulate matter, sulfur oxide (SO_{X}), nitrogen oxides (NO_{C}), and carbon monoxide (CO). In a further aspect, the proportion of CO₂ in flue gas varies by the combustion source. For instance, coal-fired power plants typically emit flue gas with approximately 12-15% CO₂; natural gas turbines emit flue gas with around 3-5% CO₂; cement plants usually exhibit higher levels of CO₂ in flue gas, around 20-25%.

Carbon Capture and Storage (CCS) is a technology designed to separate and capture CO₂, typically from large point sources, to prevent atmospheric release, storing it instead in deep geological formations. Although geological storage has the highest CO₂ storage capacity and is the most commonly industrially used technology, it requires specific geological conditions and carries potential leakage risks which could have adverse effects on groundwater quality and soil fertility. In another aspect, CCS involves energy and financial costs with the sole purpose of mitigating CO₂, posing a substantial industrial burden. In contrast, Carbon Capture and Utilization (CCU) turns CO₂ emissions from flue gas into useful resources, thereby providing long-term stability and economic benefits. CO₂ can be used as it is without chemical reaction, such as production of supercritical CO₂. CO₂ can also serve as a raw material for chemical synthesis. Recently, various methods, including biological, photocatalytic, electrochemical, and thermal approaches, have been developed to convert CO₂ into valuable chemicals. For example, CO₂ can be electrochemically converted into CO, carboxylic acid, alcohols and alkenes, and thermally catalyzed to Syngas. However, in a further aspect, each of these methods presents drawbacks such as time consumption in biological processes, low yields in photocatalysis, and high energy requirements in thermal and electrochemical methods. Notably, both CCS and CCU often require capture and concentrate CO₂ first for further treatment. For instance, solvent offers rapid kinetics and cost-efficiency, but issues such as corrosion and toxicity cannot be ignored. Solid sorbents yield minimal waste during recycling and are adaptable across temperature ranges, however, they suffer from selectivity issues, large pore sizes, and subpar adsorption efficiency. Membrane separation involves no chemical usage, and may show stable performance over extended periods, however, its industrial scalability is limited by challenges in selectivity, permeability, and durability.

Post-combustion carbon capture (PCCC), a mature technology with established operational experience that captures CO₂ from point sources such as power plants, offers a more economical and feasible approach. Its widespread adoption across various industries underscores its reliability and effectiveness in CO₂ capture. Moreover, PCCC systems can be integrated into existing industrial facilities with minimal modifications and typically operate under near-ambient conditions.

To address the limitations of CCS and CCU, the present disclosure in one aspect identifies a method that is both efficient and cost-effective while being ecologically sound. Thus, disclosed herein is a technique for converting CO₂ from flue gas into CaCO₃ nanoparticles using glycinate-based solvent. It was hypothesized that CaCO₃ nanoparticles with consistent shape and size can be produced from flue gas with varying CO₂ concentrations by tuning the gas bubbling time and flow rate to achieve the same CO₂ loading and carbamate levels in the solvent. In some aspects, the solvent includes, but is not limited to, water.

In a further aspect, the CaCO₃ nanoparticles produced by the disclosed method may partially offset CO₂ management cost as CaCO₃ nanoparticles has been wildly used as fillers in plastics, rubbers, and coatings, carriers in drug delivery, and absorbents for water and air purification.

In one aspect, amines are the most widely used solvents for CO₂ capture. Compared to strong bases like NaOH, amines offer advantages such as lower corrosiveness, higher CO₂ selectivity, and significantly reduced energy requirements for regeneration. Monoethanolamine is a commonly used amine in industrial applications, but it has notable drawbacks, including poor stability (degrading into formate, acetate, and sulfate), high evaporation losses, and toxicity. In another aspect, strong bases like NaOH have high CO₂ absorption capabilities, but their considerable corrosiveness and complexity/challenges in recovery limit their practical applications.

In one aspect, the CO₂ captured is typically sequestered underground, which remains the most widely used method at scale. However, environmental risks and limited long-term stability raise concerns. Efforts to convert CO₂ into valuable products like formic acid, methanol, or other chemicals using electrochemical, thermal, or photochemical methods face challenges of high energy costs and low yields, impeding commercialization. Carbon mineralization, which involves converting CO₂ into stable carbonate minerals, is a promising approach due to its thermodynamic stability and environmental benefits. However, challenges such as mass transfer limitations and economic viability must be addressed. In an aspect, disclosed herein is a new approach that increases the molar ratio of amino acid to base in the amino acid salt solvent formulation. In an aspect, and without wishing to be bound by theory, this strategy offers several advantages: 1) pH control: The excess amino acid reduces the solvent pH, maintaining a pH of 8-10 and mitigating corrosion risks. 2) Tunable mineralization: Amino acids influence the nucleation and growth of mineral particles, potentially allowing for controlled product morphology. 3) Value-added products: By producing CaCO₃ nanoparticles, which have a significantly higher market value than large sized CaCO₃, the economic feasibility of the process is improved.

In one aspect, the disclosed process began with a glycine (Gly)-based salt solvent that was exposed to flue gas, enabling the selective absorption of CO₂. Subsequent addition of CaCl₂) facilitated the mineralization of the absorbed CO₂ into CaCO₃ nanoparticles. This technique has been confirmed to be suitable for flue gases with varying CO₂ concentrations. The mechanism for controlling the size and shape of CaCO₃ nanoparticles was discussed. Nanotoxicity is a worldwide concern, hence BEAS-2B cells derived from normal bronchial epithelial cells were exposed to the CaCO₃ nanoparticles to evaluate the potential health risk in industrial production.

Conventionally, CaO-based solid sorbents with cyclic CO₂ absorption-desorption processes are well studied for CO₂ management, relying on the following reversible carbonation and calcination reactions:

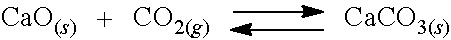

The above calcination process usually takes place at high temperature (e.g., 700° C.) and the carbonation reaction is a typical non-catalytic gas-solid reaction that occurs in two stages: an initial fast reaction stage followed by a substantially slower second reaction stage which is controlled by CO₂ diffusion through the CaCO₃ layer formed at the initial stage. As a result, the total carbonation time has been unrealistically long for any industrial applications therefore limiting the use of CaO sorbents in industry. To overcome such a key challenge related to CaO sorbents, In one aspect, herein is proposed an integrated CO₂ capture and conversion nanotechnology to directly convert CO₂ from flue gases into value-added carbonate nanomaterials. Integration of CO₂ capture and conversion may be cost- and energy-effective and may bypass limiting chemical equilibria to enable reactions to occur at low temperatures and pressures. Different from CaO solid sorbents, in this study, CaO is dissolved in H₂O, providing gas-liquid interface for CaO—CO₂ reactions to occur quickly. In a further aspect, the reactant CaO can be obtained from various natural sources like limestone; it has a low price and high availability, and various industrial alkaline residues are rich in CaO. In still another aspect, the product CaCO₃ is environmentally benign, and CaCO₃ nanoparticles have been widely used in industries such as construction, rubber, plastics, and paints to enhance properties like strength, durability, and resistance.

In some aspects, the disclosed process involved three steps: 1) Solvent preparation: Gly and NaOH were dissolved at specific molar ratios to prepare the solvent. 2) CO₂ mineralization: CaO was added to the solvent to produce CaCO₃ nanoparticles while regenerating the amino acid salt solvent. 3) Solvent recycling and reuse: The regenerated amino acid salt solvent was reused in multiple cycles to maximize cost efficiency. It was demonstrated that increasing the amino acid-to-base ratio effectively controls pH, enhances CO₂ mineralization, and yields high-valued CaCO₃ nanoparticles under industrially relevant conditions. The use of common raw materials and facilities further underscores the practicality and scalability of this method.

In one aspect, the present disclosure overcomes some limitations of the commonly used method for carbon storage (i.e., geological CO₂ sequestration/storage), which requires specific geological conditions and carries potential leakage risks and high energy consumption.

In another aspect, the disclosed carbonate nanomaterials are produced using amino acids, while the shapes of the nanomaterials can be tuned by the use of different amino acids. In a further aspect, the amino acids are not consumed while performing the method and can be re-used. Also disclosed are one- and two-step methods for producing the nanomaterials. In a further aspect, various carbonate nanomaterials with different metal cations are contemplated and should be considered disclosed, including, but not limited to, CaCO₃, BaCO₃, MgCO₃, SrCO₃, Ag₂CO₃, and combinations thereof.

In one aspect, CaCO₃ nanoparticles can be used in the plastics, rubber, cement, paint, and paper industries as enhancers and fillers. In another aspect, BaCO₃ nanoparticles can be used in the manufacture of electronic ceramic materials such as, for example, high dielectric constant ceramics in capacitors and inductors. In a further aspect, Ag₂CO₃ nanomaterials can be used in antibacterial coatings and medical materials. In a still further aspect, SrCO₃ nanomaterials can be used in the manufacture of certain drugs such as, for example, those for treating osteoporosis.

### Carbon Capture and Climate Change

Carbon dioxide (CO₂), the chief culprit of global warming, has been dramatically increasing, directly threatening the living environment of humans and animals. To keep global warming to no more than 1.5° C. higher than that of pre-industry—as called for in the Paris Agreement—emissions need to be reduced by 45% by 2030 and to reach net zero by 2050. In one aspect, converting CO₂ into nano-carbonates can help address this problem, since such a conversion process is thermodynamically favored—the Gibbs energy of carbonates is lower than that of CO₂, making the reaction energetically favorable.

Carbon Capture, Utilization, and Storage (CCUS) is the most common technology designed to capture and address CO₂ emissions, typically from large point sources (e.g., power plants). CO₂ is stored in oil and gas fields, coal beds, salt caverns or aquifers, or can be used as feedstock to be converted into value-added chemicals (e.g., methanol, ethanol) or used as-is (e.g., as supercritical CO₂ or as a raw material for chemical synthesis). The storage of CO₂ faces significant industrial challenges, including the need for specific sites and the risk of leakage. Utilizing CO₂ presents a more advantageous approach. In some cases, CO₂ can be electrochemically converted into CO, carboxylic acids, alcohols, and/or alkenes, or thermally catalyzed into Syngas. Despite this, each technology presents drawbacks such as time consumption in biological processes, low yields in photocatalysis, and high energy requirements in thermal and electrochemical methods.

Two-Step Method for Capturing CO₂ and Producing Metal Carbonate Nanoparticles: In one aspect, disclosed herein is a method for producing a metal carbonate nanoparticle, the method including at least the steps of:
(a) bubbling a gas comprising carbon dioxide through an aqueous solution containing an amino acid and a base to create a precursor solution; and
(b) admixing a metal salt into the precursor solution to form a nanoparticle solution;
wherein the metal carbonate nanoparticle precipitates from the nanoparticle solution following step (b).

In another aspect, the gas is concentrated CO₂ or is a gas mixture containing CO₂, such as, for example, real flue gas (RFG), simulated flue gas (SFG), air, or any combination thereof. In an aspect, the gas mixture includes from about 0.01% to about 100% CO₂, or from about 0.5% to about 15% CO₂.

In an aspect, the amino acid can be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof. In some aspects, non-proteinogenic amino acids can also be used. In another aspect, the amino acid is present in the precursor solution at a concentration of from about 0.1 M to about 5 M, or from about 1 M to about 2 M.

In still another aspect, the base can be sodium hydroxide (NaOH), potassium hydroxide (KOH), barium hydroxide (Ba(OH)₂), lithium hydroxide (LiOH), calcium hydroxide (Ca(OH)₂), Mg(OH)₂, Mn(OH)₂, Fe(OH)₂, AI(OH)₃, NH₄OH, K₂CO₃, KHCO₃, Na₂CO₃, NaHCO₃, or any combination thereof, and can be present in the precursor solution at a concentration of from about 0.1 M to about 5 M, or from about 1 M to about 2 M. Although certain bases are listed herein, other hydroxide bases are contemplated and should be considered disclosed.

In another aspect, the metal salt can be CaCl₂), BaCl₂, SrCl₂, MgCl₂, AgNO₃, Ca(NO₃)₂, Ba(NO₃)₂, Ag₂SO₄, CaO, BaO, SrO, MgO, Ag₂O, ZnO, Fe₂O₃, CuO, CaCl₂), Ca(NO₃)₂, Ca(CH₃COO)₂, CaBr₂, another water soluble salt of calcium, barium, strontium, magnesium, or silver, an oxide of one of these, or any combination thereof, and can be admixed into the precursor solution in a concentration of from about 0.1 M to about 5 M, or about 1 M to about 2 M.

In yet another aspect, the metal carbonate nanoparticles can be CaCO₃, BaCO₃, SrCO₃, MgCO₃, Ag₂CO₃, ZnCO₃, FeCO₃, CuCO₃, PbCO₃, CdCO₃, or any combination thereof. In one aspect, the metal carbonate has a low solubility in the solvent medium, which includes, but is not limited to, water. In a further aspect, the metal carbonate nanoparticles can have a diameter of from about 10 nm to about 150 nm, or of from about 25 nm to about 150 nm, or from about 50 nm to about 150 nm. In some aspects, the diameter can be lower than 10 nm or higher than 150 nm. In one aspect, the metal carbonate nanoparticles have a spherical morphology, a rod-like morphology, a fiber morphology, or a tube morphology. In another aspect, when the metal carbonate nanoparticles have a rod-like morphology, the metal carbonate nanoparticles can have a diameter-to-length ratio of from about 1:1 to about 1:6. However, other diameter-to-length ratios are also contemplated and should also be considered disclosed.

In one aspect, in the two-step method, nanoparticles can be removed by centrifugation, decanting, or filtration. Further in this aspect, the amino acid and/or other water-soluble compounds remain in the supernatant or filtrate solution, which can be re-used to perform the method one or more additional times.

One-Step Method for Capturing CO₂ and Producing Metal Carbonate Nanoparticles: In another aspect, disclosed herein is a method for producing a metal carbonate nanoparticle, the method including at least the step of bubbling a gas containing carbon dioxide through an aqueous solution comprising an amino acid, a base, and a metal salt to form a nanoparticle solution. Further in this aspect, the base and the metal salt can be the same compound (e.g. Ba(OH)₂).

In another aspect, the gas is concentrated CO₂ or is a gas mixture containing CO₂, such as, for example, real flue gas (RFG), simulated flue gas (SFG), air, or any combination thereof. In an aspect, the gas mixture includes from about 0.01% to about 100% CO₂, or from about 4% to about 20% CO₂, or from about 0.5% to about 15% CO₂.

In an aspect, the amino acid can be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof. In some aspects, non-proteinogenic amino acids can also be used. In another aspect, the amino acid is present in the aqueous solution at a concentration of from about 0.1 M to about 5 M, or from about 1 M to about 2 M. In some aspects, for higher concentrations, the concentration may be limited by amino acid solubility, which can vary by amino acid. In other aspects, low concentrations can be used but may result in a limited production of nanoparticles.

In still another aspect, the base can be sodium hydroxide (NaOH), potassium hydroxide (KOH), barium hydroxide (Ba(OH)₂), lithium hydroxide (LiOH), calcium hydroxide (Ca(OH)₂), Mg(OH)₂, Mn(OH)₂, Fe(OH)₂, AI(OH)₃, NH₄OH, K₂CO₃, KHCO₃, Na₂CO₃, NaHCO₃, or any combination thereof, and can be present in the aqueous solution at a concentration of from about 0.1 M to about 5 M, or from about 1 M to about 2 M. In some aspects, bases that have less solubility in water (e.g., Ca(OH)₂) may perform better in the two-step process as opposed to the one-step process. In another aspect, other metal hydroxides can also be used as the base. In some aspects, the amino acid and the base are present in the aqueous solution in a molar ratio of from about 3:1 to about 1:1.

In another aspect, the metal salt can be CaCl₂), BaCl₂, SrCl₂, MgCl₂, AgNO₃, ZnCO₃, FeCO₃, CuCO₃, PbCO₃, CdCO₃, or any combination thereof, or another carbonate that has low solubility in the solvent medium, which can be water or another solvent, and can be admixed into the aqueous solution in a concentration of from about 0.1 M to about 5 M, or about 1 M to about 2 M.

In some aspects, the metal salt and the base are the same compound (e.g. barium hydroxide (Ba(OH)₂), calcium hydroxide (Ca(OH)₂), or the like).

In yet another aspect, the metal carbonate nanoparticles can be CaCO₃, BaCO₃, SrCO₃, MgCO₃, Ag₂CO₃, or any combination thereof and have a diameter of from about 10 nm to about 150 nm, or of from about 25 nm to about 150 nm, or of from about 50 nm to about 150 nm. In some aspects, the diameter can be less than 10 nm or larger than 150 nm. In one aspect, the metal carbonate nanoparticles have a spherical morphology or a rod-like morphology, or can be fibers or tubes. In another aspect, when the metal carbonate nanoparticles have a rod-like morphology, the metal carbonate nanoparticles can have a diameter-to-length ratio of from about 1:1 to about 1:6. However, other diameter-to-length ratios are contemplated and should also be considered disclosed.

In some aspects, the aqueous solution has a pH of from about 8 to about 12, or from about 8 to about 10. In a further aspect, the pH of the aqueous solution causes no to minimal corrosion of a reactor containing the aqueous solution. In still another aspect, bubbling can be conducted at a temperature of from about 25° C. to about 60° C. In some aspects, the bubbling can be done at a temperature below 25° C. or above 60° C.

Recovery and Re-Use of Amino Acids: In some aspects, the method further includes removing the metal carbonate nanoparticles from the nanoparticle solution by a method such as, for example, centrifugation, decanting, or filtration. In a further aspect, a supernatant or filtrate comprising the amino acid is retained following centrifugation, decanting, or filtration and can be re-used in the disclosed method one or more times. Further in this aspect, the amino acid, which is soluble in water, will remain in solution, and the whole solution can be re-used after solids (i.e., nanoparticles) are removed.

### Applications of Carbonate Nanomaterials

Also disclosed herein are metal carbonate nanoparticles produced by the disclosed methods, and articles or compositions including the metal carbonate nanoparticles. The articles and compositions have numerous uses across a variety of industries, a non-exhaustive number of which are discussed below.

Medical Applications: In an aspect, the article or composition can be a drug, a drug-incorporated product, a wound care product, an engineered tissue, a surface coating, or any combination thereof. Wound infection and antibiotic resistance have become worldwide healthcare burdens, leading to high morbidity and mortality. According to the Centers for Disease Control and Prevention (CDC), infection from antibiotic-resistant “superbugs” affect nearly three million people and kill at least 35,000 people annually in the US. Nanoparticles have arisen as an alternative to traditional therapies and have been widely used for antibacterial treatments. Among them, silver nanoparticles (AgNPs) are the most widely explored, as they have shown robust antimicrobial effectiveness against various bacteria, viruses, and fungi. AgNPs can be applied to reduce infections caused by various microorganisms as well as to decontaminate medical supplies. Potential mechanisms of action of AgNPs include: 1) binding to cell membrane, membrane proteins, and DNA bases, 2) releasing Ag⁺ and 3) generating reactive oxidative species. Ag₂CO₃ nanoparticles produced from the CO₂ conversion methods described herein can reduce infection and may lead to improved clinical outcomes compared to the conventional AgNPs; the use of Ag₂CO₃ nanoparticles also affects the local pH, leading toward improved elimination of pathogens.

Wounds affect over 6 million people annually in the US at a cost of $25 billion. Wound infections are of particular concern. However, strategies for treating wound infections remain challenging due to the occurrence of biofilm colonization, delayed healing, and drug resistance.

Nanomaterials, recognized for their potential to dramatically alter the properties of bulk materials, are typically characterized by having at least one dimension smaller than 100 nm. Due to their distinctive properties, such as high surface area, reactivity, and customizability for specific functions, nanomaterials have become a focal point of scientific research and application across various fields.

CO₂ has previously been shown to enhance wound recovery, including recovery of skin wounds caused by accidental damage or surgery. Accelerating wound healing has recently become one of the most important aspects in terms of wound treatment, especially for the non-healing wound. Recent studies have revealed that local hypoxia caused by insufficient microcirculatory blood flow is one reason for wounds not healing properly, and adequate O₂ supply is a prerequisite for the growth and remodeling of wound tissue. Hence, in one aspect, increasing the O₂ content around wounds could promote healing. Expensive equipment and professional operation requirements for oxygen administration, as well as the risk of O₂ poisoning, limits the wide application of O₂ therapies for wound healing treatment. Clinical studies through transcutaneous or subcutaneous administration of CO₂ have demonstrated the efficacy in improving microcirculation and the rise of O₂ level due to the Bohr effect. Compared to the direct use of O₂, CO₂ acts more conveniently on the wound due to its high solubility in body fluid. Elevating tissue CO₂ levels pushes the tissue environment toward “thrive mode,” bringing increased blood flow and O₂, reduced inflammation, and enhanced angiogenesis. Hence, instead of the direct use of O₂ for wound treatment, CO₂ could be a useful alternative. However, the currently available CO₂ treatments are limited to direct administration of CO₂ gas through transdermal or subcutaneous delivery conducted by a device equipped with CO₂ gas, which may not be accessible in every clinic. For a course of treatment, multiple administrations may be needed to deliver CO₂ into wounds, causing the disadvantages of being time-consuming, increasing the risk of injection site infection, and causing pain associated with multiple injections. The potential risk of overdose causing CO₂ poisoning must also be considered. Therefore, on-demand and controlled CO₂ release at the wound site is important. Metal carbonates synthesized via the methods described here are used for local applications for controlled CO₂ release due to the dissociation of the carbonates in the biological environment, thereby enhancing wound recovery.

In an aspect, the drug can be an antibiotic, an antitumor agent, an antiviral agent, an osteoporosis drug, or a stimulator of angiogenesis. Further in this aspect, the drug can be an osteoporosis drug and wherein the metal carbonate nanoparticles can be or include SrCO₃. In another aspect, when the metal carbonate nanoparticles are or include Ag₂CO₃, the wound care product comprises a bandage, a gauze, a film, a foam, a hydrogel, or any combination thereof. In a further aspect, the disclosed wound healing materials and compositions can also stimulate angiogenesis.

Industrial Applications: In an aspect, the article or composition can be a filler, a catalyst, an electronic component, a ceramic material, a surface coating, or any combination thereof.

In an alternative aspect, the metal carbonate nanoparticles can be or include CaCO₃ and the filler can be a plastic, cement, rubber, paint, or paper filler. In still another aspect, the metal carbonate nanoparticles can be or include BaCO₃ and the ceramic material can be a high dielectric constant ceramic material. Further in this aspect, when the metal carbonate nanoparticles include BaCO₃ and the electronic component can be a capacitor or an inductor made from the high dielectric constant ceramic material.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an,” and “the” include plural referents unless the context clearly dictates otherwise. Thus, for example, reference to “a metal ion,” “a base,” or “an amino acid,” includes, but is not limited to, mixtures or combinations of two or more such metal ions, bases, or amino acids, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an amino acid refers to an amount that is sufficient to achieve the desired improvement in the property modulated by the formulation component, e.g., achieving the desired level of carbon dioxide capture and conversion to carbonate nanoparticles. The specific level in terms of wt % in a composition required as an effective amount will depend upon a variety of factors including the concentration and type of amino acid, CO₂ content of the gas mixture from which carbon capture occurs, concentration and identity of base used, and end use of the nanoparticles made according to the disclosed method.

As used herein, the term “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere). However, in one aspect, the present methods and systems can be active at pressures other than one atmosphere.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A method for producing a metal carbonate nanoparticle, the method comprising:
(a) bubbling a gas comprising carbon dioxide through an aqueous solution comprising an amino acid and a base to create a precursor solution; and
(b) admixing a metal salt into the precursor solution to form a nanoparticle solution;
wherein the metal carbonate nanoparticle precipitates from the nanoparticle solution following step (b).

Aspect 2. The method of aspect 1, wherein the gas comprises concentrated CO₂ or a gas mixture comprising CO₂.

Aspect 3. The method of aspect 2, wherein the gas mixture comprising CO₂ comprises real flue gas (RFG), simulated flue gas (SFG), air, or any combination thereof.

Aspect 4. The method of aspect 2 or 3, wherein the gas mixture comprises from about 0.01% to 100% CO₂.

Aspect 5. The method of any one of aspects 1-4, wherein the amino acid comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof.

Aspect 6. The method of aspect 5, wherein the amino acid is alanine, asparagine, or cysteine.

Aspect 7. The method of any one of aspects 1-6, wherein the amino acid is present in the precursor solution at a concentration of from about 0.1 M to about 5 M.

Aspect 8. The method of any one of aspects 1-7, wherein the base comprises sodium hydroxide (NaOH), potassium hydroxide (KOH), barium hydroxide (Ba(OH)₂), lithium hydroxide (LiOH), Mg(OH)₂, Mn(OH)₂, Fe(OH)₂, Al(OH)₃, NH₄OH, K₂CO₃, KHCO₃, Na₂CO₃, NaHCO₃, or any combination thereof.

Aspect 9. The method of aspect 8, wherein the base is NaOH or KOH.

Aspect 10. The method of any one of aspects 1-9, wherein the base is present in the precursor solution at a concentration of from about 0.1 M to about 5 M.

Aspect 11. The method of any one of aspects 1-10, wherein the metal salt comprises CaCl₂), BaCl₂, SrCl₂, MgCl₂, AgNO₃, Ca(NO₃)₂, Ba(NO₃)₂, Ag₂SO₄, CaO, BaO, SrO, MgO, Ag₂O, ZnO, Fe₂O₃, CuO, CaCl₂), Ca(NO₃)₂, Ca(CH₃COO)₂, CaBr₂, or any combination thereof.

Aspect 12. The method of aspect 11, wherein the metal salt is CaCl₂), BaCl₂, or AgNO₃.

Aspect 13. The method of any one of aspects 1-12, wherein the metal salt is admixed into the precursor solution in a concentration of from about 0.1 M to about 5 M.

Aspect 14. The method of any one of aspects 1-13, wherein the metal carbonate nanoparticles comprise CaCO₃, BaCO₃, SrCO₃, MgCO₃, Ag₂CO₃, ZnCO₃, FeCO₃, CuCO₃, PbCO₃, CdCO₃, or any combination thereof.

Aspect 15. The method of aspect 14, wherein the metal carbonate nanoparticles are CaCO₃, BaCO₃, SrCO₃, or Ag₂CO₃.

Aspect 16. The method of any one of aspects 1-15, wherein the metal carbonate nanoparticles have a diameter of from about 10 nm to about 150 nm.

Aspect 17. The method of aspect 16, wherein the metal carbonate nanoparticles have a diameter of from about 25 nm to about 150 nm.

Aspect 18. The method of any one of aspects 1-17, wherein the metal carbonate nanoparticles have a spherical morphology, a rod-like morphology, a fiber morphology, or a tube morphology.

Aspect 19. The method of aspect 18, wherein the metal carbonate nanoparticles have a rod-like morphology and a diameter-to-length ratio of from about 1:1 to about 1:6.

Aspect 20. A method for producing a metal carbonate nanoparticle, the method comprising: bubbling a gas comprising carbon dioxide through an aqueous solution comprising an amino acid, a base, and a metal salt to form a nanoparticle solution.

Aspect 21. The method of aspect 20, wherein the gas comprises concentrated CO₂ or a gas mixture comprising CO₂.

Aspect 22. The method of aspect 21, wherein the gas mixture comprising CO₂ comprises real flue gas (RFG), simulated flue gas (SFG), air, or any combination thereof.

Aspect 23. The method of aspect 21 or 22, wherein the gas mixture comprises from about 0.01% to 100% CO₂.

Aspect 24. The method of aspect 23, wherein the gas mixture comprises from about 4% to about 20% CO₂.

Aspect 25. The method of any one of aspects 20-24, wherein the amino acid comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof.

Aspect 26. The method of aspect 25, wherein the amino acid is alanine, asparagine, or cysteine.

Aspect 27. The method of any one of aspects 20-26, wherein the amino acid is present in the aqueous solution a concentration of from about 0.1 M to about 5 M.

Aspect 28. The method of any one of aspects 20-27, wherein the base comprises sodium hydroxide (NaOH), potassium hydroxide (KOH), barium hydroxide (Ba(OH)₂), lithium hydroxide (LiOH), or any combination thereof.

Aspect 29. The method of aspect 28, wherein the base is NaOH or KOH.

Aspect 30. The method of any one of aspects 20-29, wherein the base is present in the aqueous solution a concentration of from about 0.1 M to about 5 M.

Aspect 31. The method of any one of aspects 20-30, wherein the amino acid and the base are present in the aqueous solution in a molar ratio of from about 3:1 to about 1:1.

Aspect 32. The method of any one of aspects 20-31, wherein the metal salt comprises CaCl₂), BaCl₂, SrCl₂, MgCl₂, AgNO₃, or any combination thereof.

Aspect 33. The method of aspect 32, wherein the metal salt is CaCl₂), BaCl₂, or AgNO₃.

Aspect 34. The method of any one of aspects 20-33, wherein the metal salt is admixed into the aqueous solution in a concentration of from about 0.1 M to about 5 M.

Aspect 35. The method of aspect 20, wherein the base and the metal salt are the same compound.

Aspect 36. The method of aspect 35, wherein the base and the metal salt comprise barium hydroxide or calcium hydroxide.

Aspect 37. The method of aspect 35 or 36, wherein the base and the metal salt that are the same compound are admixed into the aqueous solution in a concentration of from about 0.1 M to about 5 M.

Aspect 38. The method of any one of aspects 20-37, wherein the metal carbonate nanoparticles comprise CaCO₃, BaCO₃, SrCO₃, MgCO₃, Ag₂CO₃, or any combination thereof.

Aspect 39. The method of aspect 38, wherein the metal carbonate nanoparticles are CaCO₃, BaCO₃, SrCO₃, or Ag₂CO₃.

Aspect 40. The method of any one of aspects 20-39, wherein the metal carbonate nanoparticles have a diameter of from about 10 nm to about 150 nm.

Aspect 41. The method of aspect 40, wherein the metal carbonate nanoparticles have a diameter of from about 25 nm to about 150 nm.

Aspect 42. The method of any one of aspects 20-41, wherein the metal carbonate nanoparticles have a spherical morphology, a rod-like morphology, a fiber morphology, or a tube morphology.

Aspect 43. The method of aspect 42, wherein the metal carbonate nanoparticles have a rod-like morphology and a diameter-to-length ratio of from about 1:1 to about 1:6.

Aspect 44. The method of any one of aspects 20-43, wherein the aqueous solution has a pH of from about 8 to about 12.

Aspect 45. The method of aspect 44, wherein the pH of the aqueous solution causes no to minimal corrosion of a reactor containing the aqueous solution.

Aspect 46. The method of any one of aspects 20-45, wherein the bubbling is conducted at a temperature of from about 25° C. to about 60° C.

Aspect 47. The method of any one of aspects 1-46, further comprising removing the metal carbonate nanoparticles from the nanoparticle solution.

Aspect 48. The method of aspect 47, wherein the metal carbonate nanoparticles are removed by centrifugation, decanting, or filtration.

Aspect 49. The method of aspect 48, wherein a supernatant or filtrate comprising the amino acid is retained following centrifugation, decanting, or filtration.

Aspect 50. The method of any one of aspects 47-49, further comprising re-using the amino acid in the supernatant or filtrate in the method.

Aspect 51. A metal carbonate nanoparticle produced by the method of any one of aspects 1-50.

Aspect 52. An article or composition comprising the metal carbonate nanoparticle of aspect 51.

Aspect 53. The article or composition of aspect 52, wherein the article or composition comprises a filler, a catalyst, a drug, an electronic component, a ceramic material, a wound care product, an engineered tissue, a surface coating, or any combination thereof.

Aspect 54. The article or composition of aspect 53, wherein the drug comprises an antibiotic, an antitumor agent, an antiviral agent, an osteoporosis drug, or a stimulator of angiogenesis.

Aspect 55. The article or composition of aspect 54, wherein the drug is an osteoporosis drug and wherein the metal carbonate nanoparticles comprise SrCO₃.

Aspect 56. The article or composition of aspect 53, wherein the metal carbonate nanoparticles comprise Ag₂CO₃ and the wound care product comprises a bandage, a gauze, a film, a foam, a hydrogel, or any combination thereof.

Aspect 57. The article or composition of aspect 53, wherein the metal carbonate nanoparticles comprise CaCO₃ and the filler comprises a plastic, cement, rubber, paint, or paper filler.

Aspect 58. The article or composition of aspect 53, wherein the metal carbonate nanoparticles comprise BaCO₃ and the ceramic material comprises a high dielectric constant ceramic material.

Aspect 59. The article or composition of aspect 53, wherein the metal carbonate nanoparticles comprise BaCO₃ and the electronic component comprises a capacitor or an inductor.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Conversion of CO₂ into CaCO₃ Nanomaterials

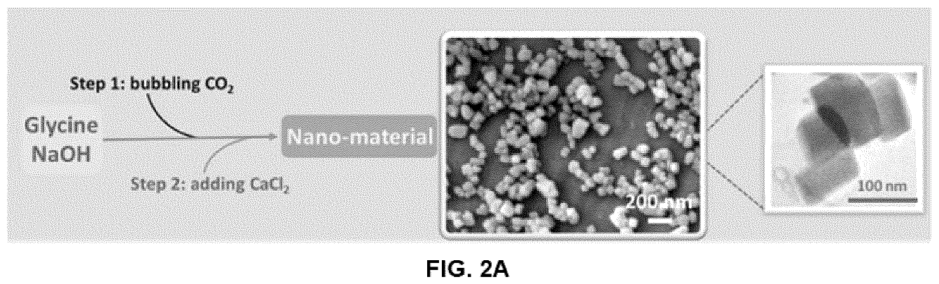

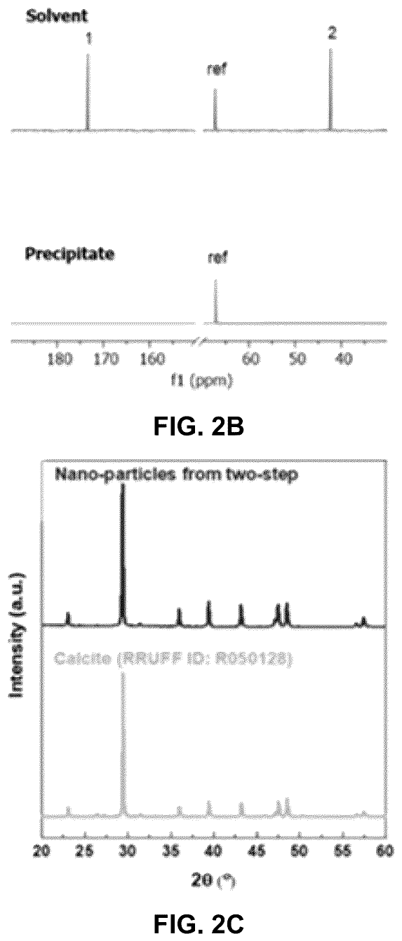

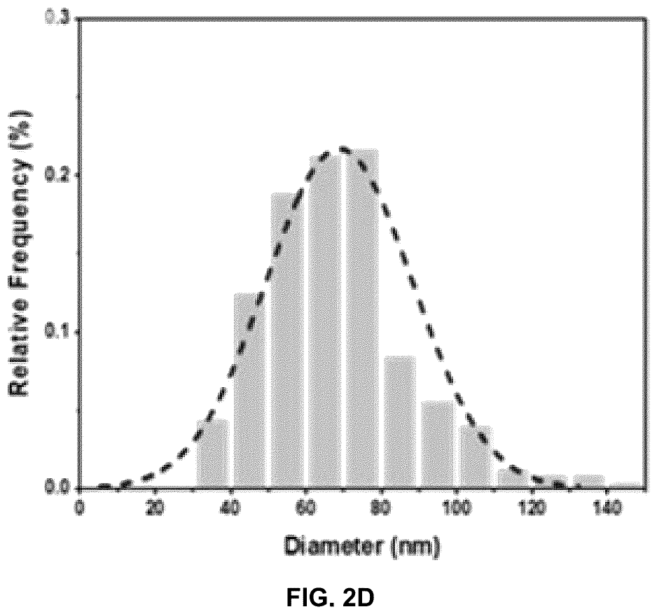

A two-step process was developed to convert CO₂ into CaCO₃ nanomaterials (FIG. 2A). In the first step, CO₂ was bubbled into an aqueous Gly salt solution. In the second step, CaCl₂) was added, which led to the formation of solid precipitates. Scanning electron microscope (SEM) and transmission electron microscopy (TEM) observations found that the precipitates were nanoparticles with sizes less than 100 nm (FIG. 2A). Nuclear magnetic resonance (NMR) data (FIG. 2B) showed no signals of organic carbon-containing species within the precipitates and only Gly was shown in the supernatant. X-ray diffraction (XRD) characterization of the precipitates found that the precipitates were pure calcite, the most stable form of CaCO₃ (FIG. 2C), and the precipitates had an average diameter of approximately 70 nm (FIG. 2D).

### Amino Acids Played a Key Role in the Competition Between the Formation of Nanoparticles and Microparticles.

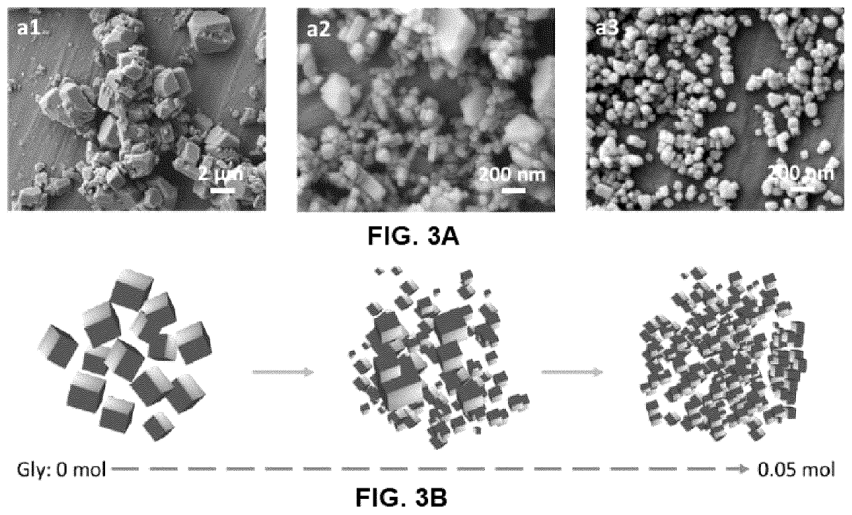

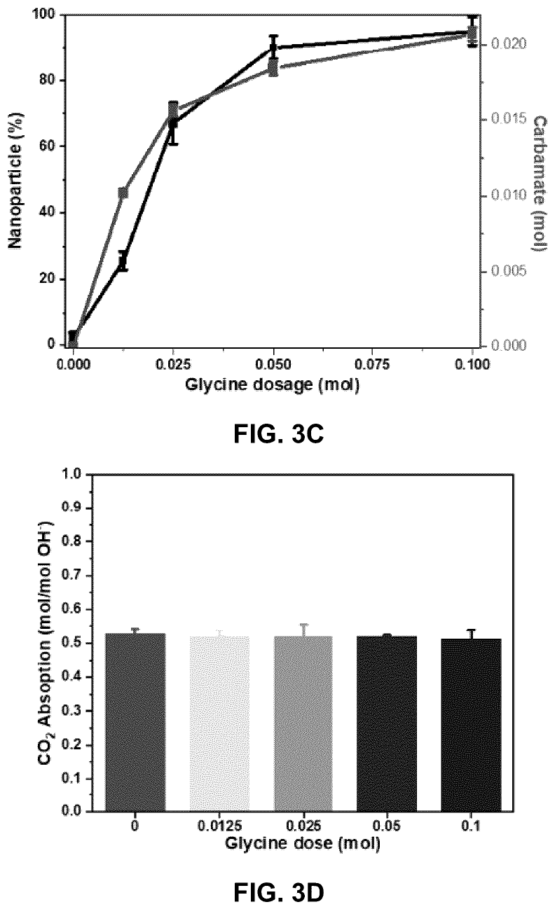

It was revealed that the morphology of particles changed dramatically with the increasing dose of Gly, as illustrated in FIG. 3A. It was shown in FIG. 3B that microparticles were formed in the absence of Gly and nanoparticles were formed at 0.05 mol Gly. Both microparticles and nanoparticles were formed and co-existed at 0.025 mol Gly (FIG. 3A), and the percentage of nanoparticles increased with increasing Gly concentration in the range of 0 to 0.10 mol Gly (FIG. 3C); note that the CO₂ conversion at 0 to 0.10 mol Gly was about the same (FIG. 3D). The formation of nanoparticles was directly correlated to the amount of carbamate formed during step 1 of the two-step process; the more carbamate formation, the more formation of nanoparticles (FIG. 3C). It was clear that in the absence of amino acids (i.e., Gly), microparticles were formed and it was the presence of Gly that led to the formation of nanoparticles. At relatively lower concentrations of Gly, there was a competition in the formation of microparticles and nanoparticles. The use of the amino acid like Gly led to the formation of CaCO₃ nanomaterials, and without the presence of Gly, CaCO₃ microparticles were obtained.

Impact of Amino Acid Type on Converting CO₂ into CaCO₃ Nanoparticles.

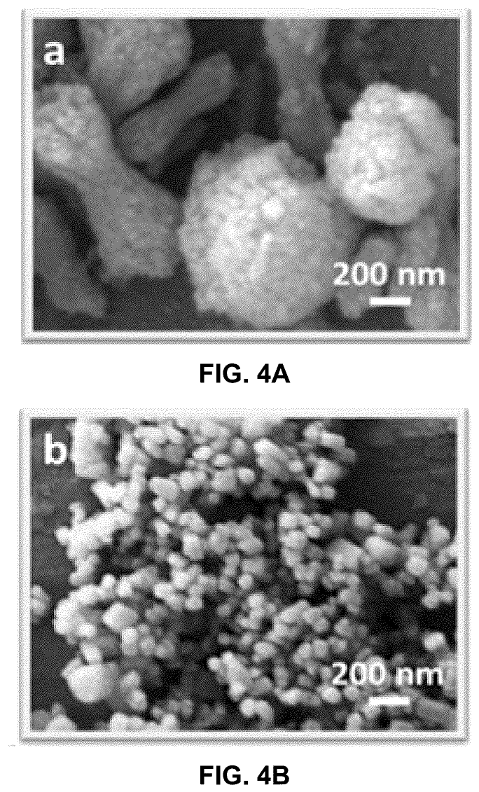

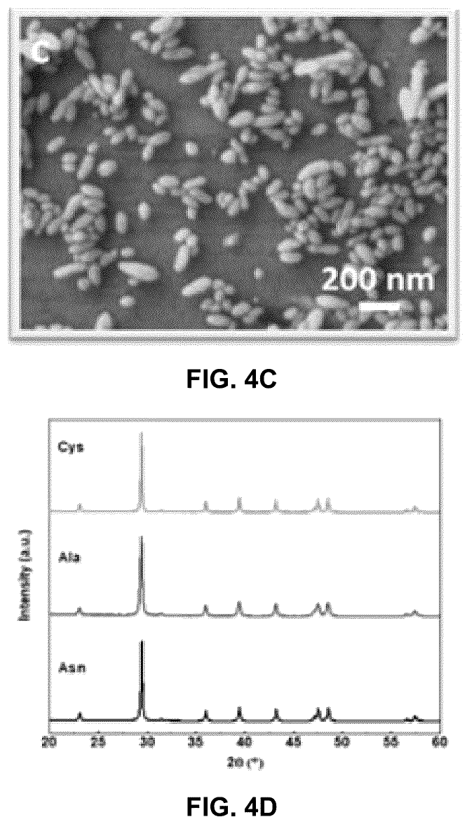

Amino acid type also influenced the conversion of CO₂ into CaCO₃. Besides Gly, three additional amino acids including asparagine (Asn), alanine (Ala), and cysteine (Cys) were assessed. These were selected to represent amino acids with polar uncharged side chains, amino acids with a hydrophobic side chain, and amino acid with unique side chains like —SH, respectively. All the amino acids studied resulted in the formation of CaCO₃ nanoparticles using the disclosed two-step process (FIGS. 4A-4D). Compared to the CaCO₃ nanoparticles from Gly (FIG. 2A), Asn and Ala formed similar shaped nanoparticles, although the nanoparticles from Asn further aggregated into micro-sized porous particles including bone-like particles (FIG. 4A) and the nanoparticles from Ala had a larger size distribution, ranging from 50 nm to 200 nm (FIG. 4B). Cys led to the formation of ellipsoidal nanoparticles whose diameter was in the range of 20-100 nm and the ratio of length to diameter was between 1 to 3 (FIG. 4C). XRD spectra data confirmed that all the nanoparticles were pure calcite (FIG. 4D).

Thus, the use of different amino acids leads to the formation of CaCO₃ nanomaterials having different morphologies.

### Synthesis Mechanism of CaCO₃ Nanomaterials.

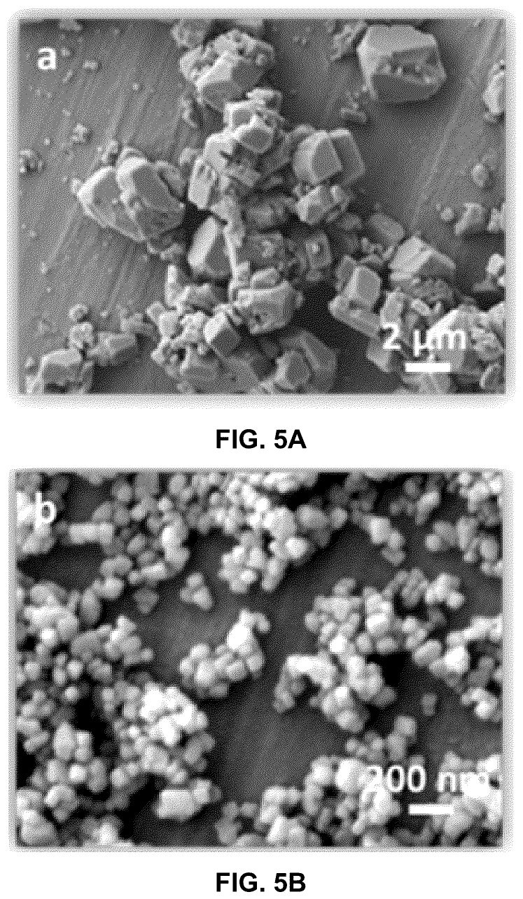

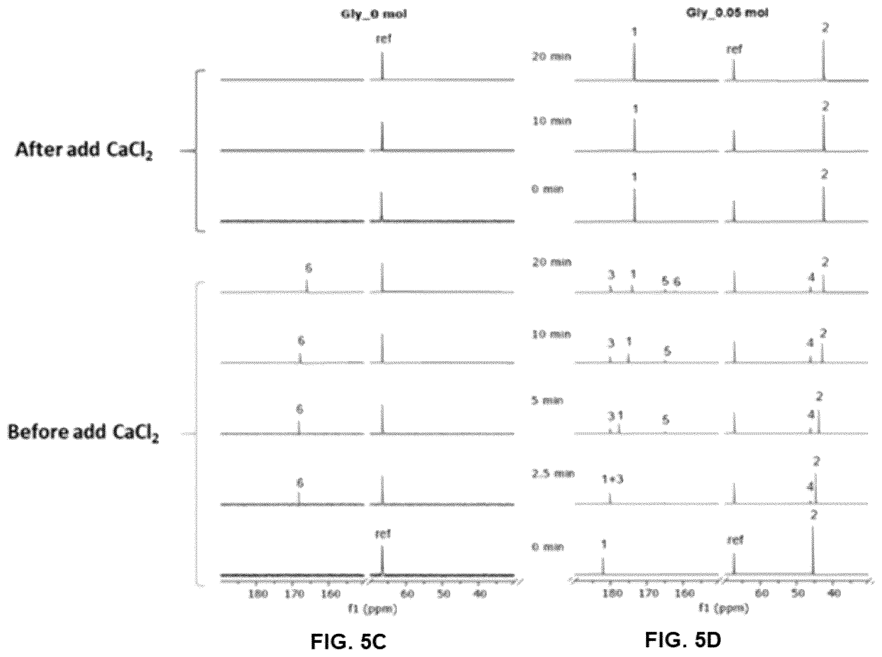

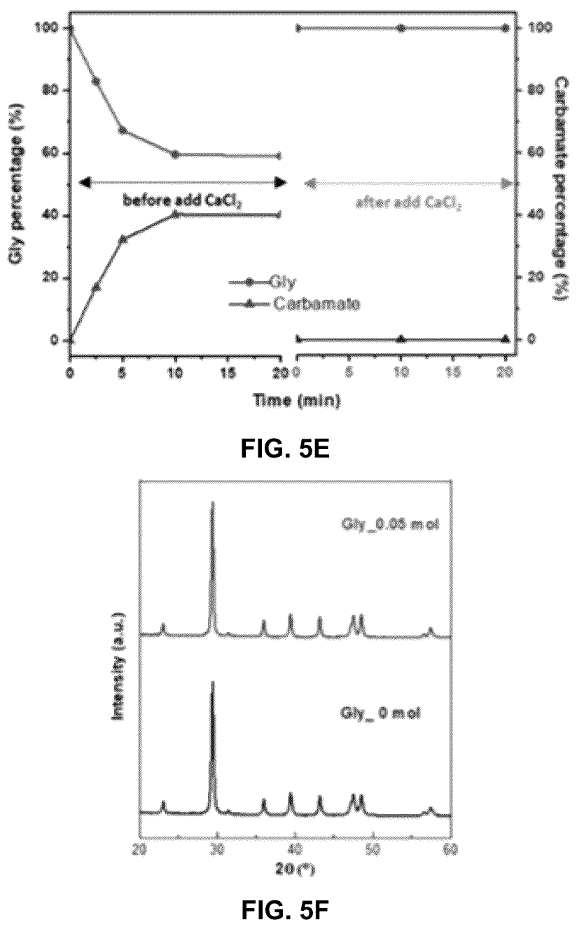

The mechanism of converting CO₂ into CaCO₃ particles in the two-step process was examined by comparing the process and final products with and without amino acids. It was found that, in the absence of Gly, micro-sized, dense CaCO₃ particles (~2 μm) were formed (FIG. 5A); in comparison, CaCO₃ nanoparticles (~70 nm) were formed in the presence of Gly (FIG. 5B). This finding suggests that Gly played a critical role in controlling the size of the final products. NMR data (FIG. 5C) showed that, in the absence of amino acids, CO₃²⁻/HCO₃⁻ was formed within 2.5 min of CO₂ bubbling and their peak position in the NMR spectra shifted slightly toward lower ppm with increasing CO₂ bubbling time in step 1, and the peak of CO₃²⁻/HCO₃⁻; disappeared immediately upon addition of CaCl₂) in step 2. In the presence of amino acids (i.e., Gly), by contrast, carbamate was first formed and CO₃²⁻/HCO₃⁻; appeared at 20 min, and upon addition of CaCl₂), both carbamate and CO₃²⁻/HCO₃⁻; disappeared and only the peaks for Gly/Gly. were observed (FIG. 5D). The percentage of carbamate increased sharply within the first 10 min and remained about the same flowed until the 20 min point in step 1, while it dropped to 0 upon addition of CaCl₂). In contrast, percentage of Gly decreased quickly within 10 min and then increased slightly at 20 min, and finally rose to 100% upon the addition of CaCl₂) (FIG. 5E). These NMR data clearly indicated that the chemical reactions during the two-step process were different between the solvents with or without amino acids (i.e., Gly), though XRD (FIG. 5F) confirmed that the converted solid products were pure calcites with or without amino acids. Meanwhile, dramatic differences in pH changes were observed between the two solvents with and without Gly. In the absence of Gly, the pH was 13.2 before CO₂ bubbling and the pH decreased with increasing CO₂ bubbling time and dropped to 10.1 at 20 min, and upon addition of CaCl₂), the pH immediately further dropped to 4.6 and no changes in pH were observed thereafter. In comparison, in the presence of Gly, the pH was 11.5 before CO₂ bubbling and it also decreased with increasing CO₂ bubbling time and became 8.7 at 20 min, and upon addition of CaCl₂), the pH immediately dropped to 5.5 and remained stable during the next 20 min. In addition, the CO₂ loading for the two solvents with and without Gly was almost identical; both were approximately 0.5 mol CO₂ loading/(mol OH⁻) after bubbling CO₂ for 20 min.

Based on the results, the following mechanism of CO₂ capture and conversion to CaCO₃ nanomaterials is proposed:

In the presence of amino acids, deprotonated Gly reacted with CO₂ to form carbamate, which subsequently hydrolyzed into HCO₃⁻. Upon addition of CaCl₂), all carbamate was hydrolyzed into HCO₃⁻ which reacted with Ca²⁺ to form CaCO₃ nanomaterials.

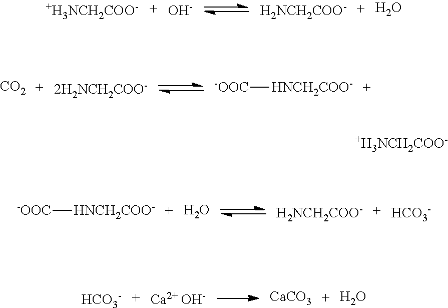

In the absence of amino acids (e.g., Gly), CO₂ reacted with OH⁻ to form HCO₃⁻ first. Upon addition of CaCl₂), HCO₃⁻; reacted with Ca²⁺ to form micro-CaCO₃⁻.

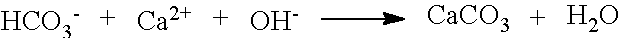

It is clear that the amino acid like Gly converted CO₂ into carbamate which further hydrolyzed into bicarbonate and led to the formation of CaCO₃ nanomaterials, and without the presence of Gly, CaCO₃ microparticles were obtained.

Other Carbonate Nanomaterials were Also Produced Via the Same Method

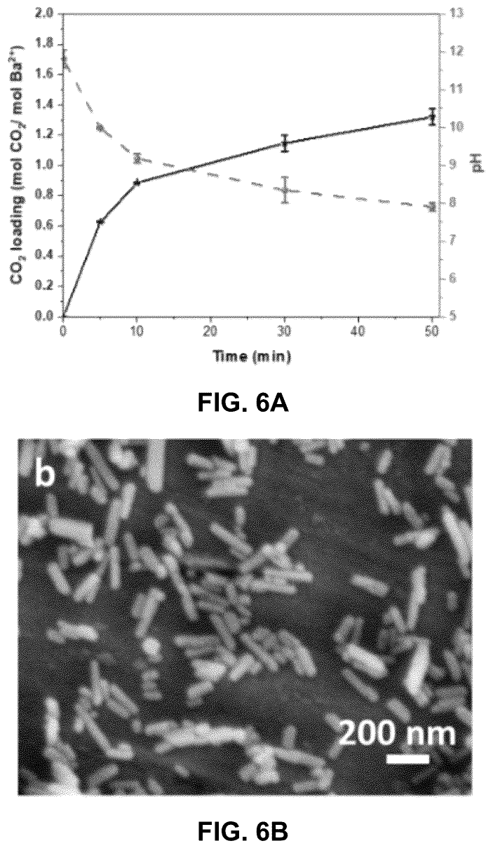

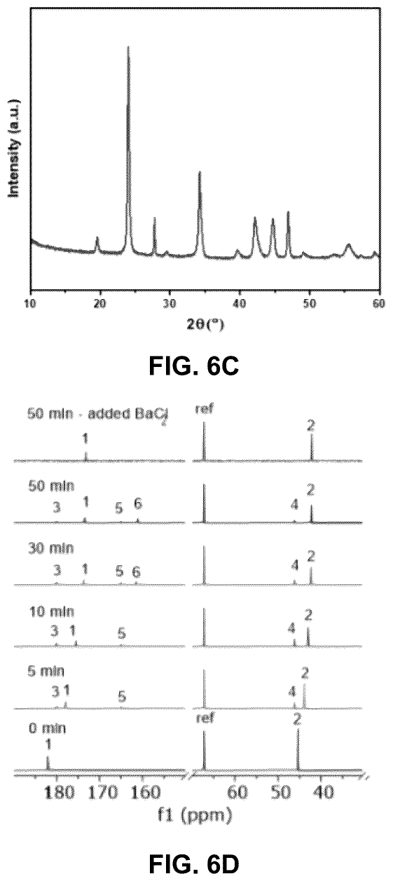

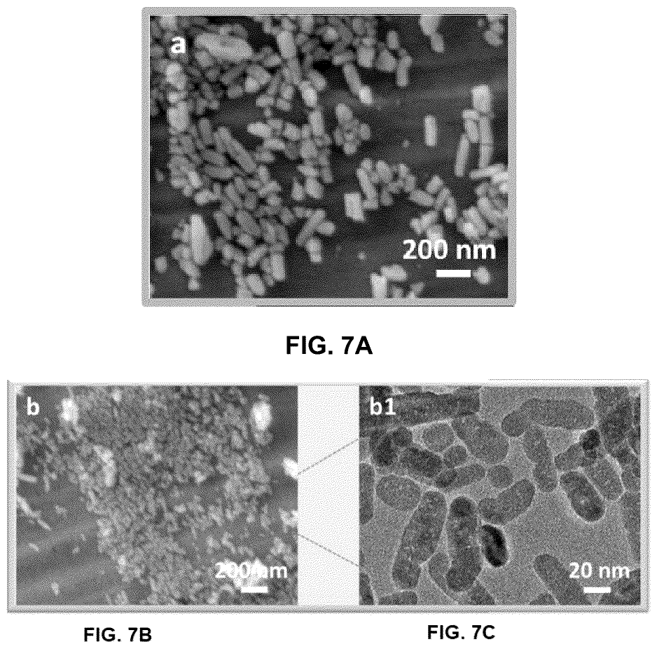

Production of BaCO₃ nanomaterials: Amino acid salts, produced by combining a strong base with an amino acid, have been widely studied for CO₂ capture due to their unique properties, such as lower evaporation rates and environmental friendliness. However, these salts typically require heating to release the captured CO₂. Inspired by the disclosed method, a process to mineralize CO₂ to BaCO₃ after bubbling CO₂ through an amino acid solution for a set duration has been proposed. These findings (FIG. 6A) revealed an increase in CO₂ loading from 0 to 50 min, reaching a maximum at approximately 1.3 mol/mol Ba²⁺ at 50 min. Concurrently, the pH of the solvent showed an inverse relationship with the CO₂ loading; as CO₂ loading increased, the pH decreased. Upon the addition of BaCl₂ at 50 min, uniformly sized particles with a diameter of ~50 nm were observed (FIG. 6B). XRD spectra (FIG. 6C) confirmed that these particles consisted of pure BaCO₃. Before adding BaCl₂, NMR (FIG. 6D) peaks (1,2) specific to Gly were detected at 0 min, and peaks (3, 4, 5) representing carbamate appeared as CO₂ was continuously bubbled into the solvent. By 30 min, peak (6) indicative of CO₃²⁻/HCO₃⁻ also emerged. After the addition of BaCl₂, peaks corresponding to carbamate (3, 4, 5) and CO₃²⁻/HCO₃⁻ (6) vanished, leaving only peaks related to Gly/Gly⁻ (1,2). These findings suggested that Gly participated in the reaction, partially converting to carbamate, and that CO₂ was effectively converted to BaCO₃ following the addition of BaCl₂. FIGS. 7A-7C show that the use of different amino acids led to the formation of BaCO₃ nanomaterials of different sizes.

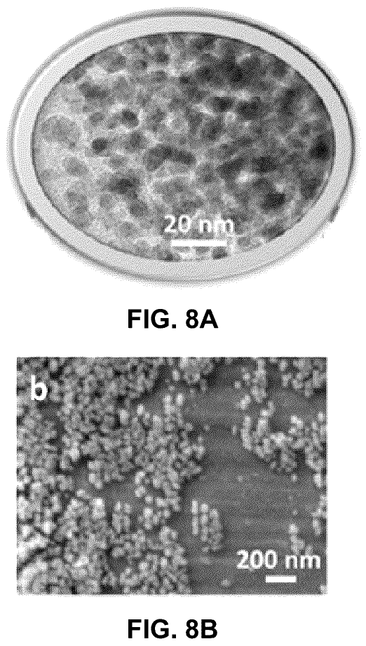

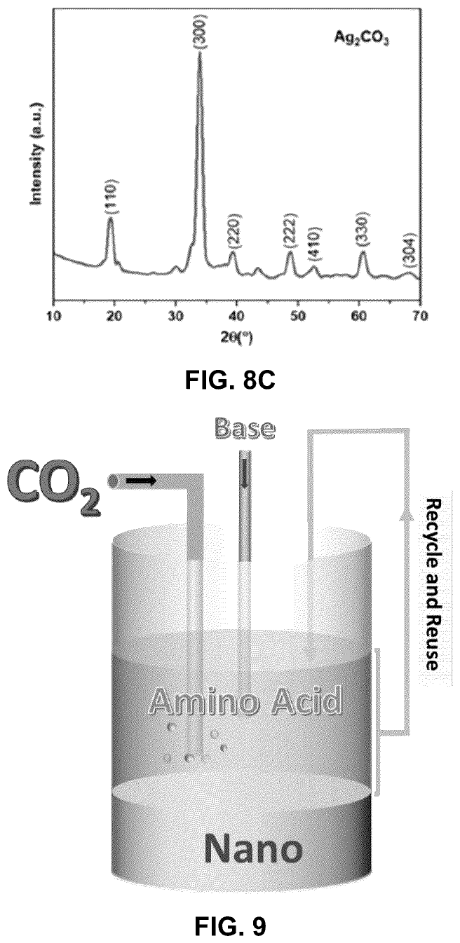

Production of Ag₂CO₃ nanomaterials: Ag₂CO₃ nanomaterials were achieved with the use of concentrated CO₂ based on the two-step method described herein. Inspired by the method and mechanism for CO₂ conversion into CaCO₃ nanomaterials, Ag₂CO₃ nanomaterials were obtained when AgNO₃ was used in the process. As shown by SEM and TEM images in FIGS. 8A-8B, the size of nanoparticles is about 10 nm. XRD spectra indicate the nanoparticles are pure Ag₂CO₃ nanomaterials (FIG. 8C).

### Example 2: One-Step Process to Convert CO₂ into Carbonate Nanomaterials

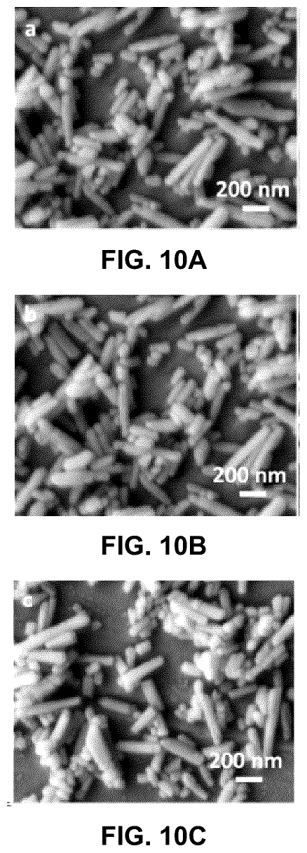

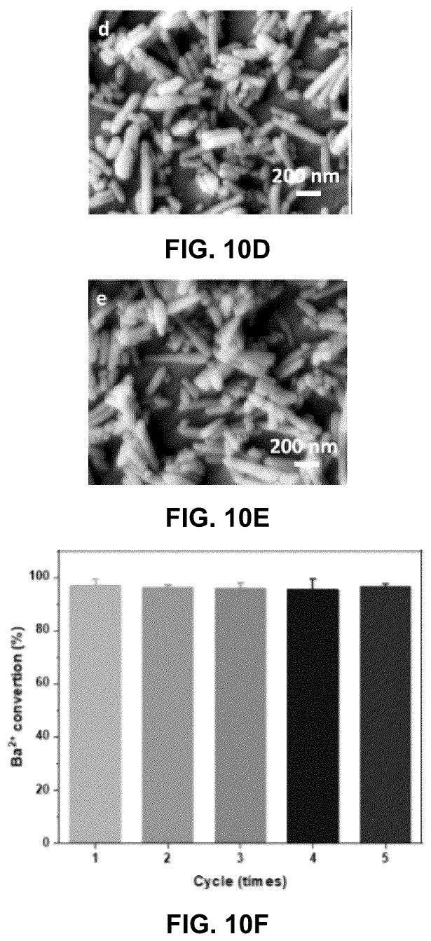

In the simplified process (FIG. 9), CO₂ or a gas mixture containing CO₂ was bubbled into a solution containing an amino acid and Ba(OH)₂. BaCO₃ nanomaterials were produced and amino acid was successfully recycled and re-used. In this case, Ba(OH)₂ is consumed and needs to be replenished for subsequent cycles. After bubbling CO₂ for 50 min in the solvent, the solid BaCO₃ precipitates are separated from the solvent via centrifugation and removed, and the remaining solvent with Gly is added with fresh Ba(OH)₂ followed by CO₂ bubbling for 50 min. This process is repeated multiple times. SEM images (FIG. 10A-10E) show that the morphology of the precipitates from the multiple cycles is similar. No significant differences in Ba²⁺ conversion are observed among the multiple cycles (FIG. 10F).

### Amino Acid Type Changed the Size and Shape of BaCO₃ Nanomaterials

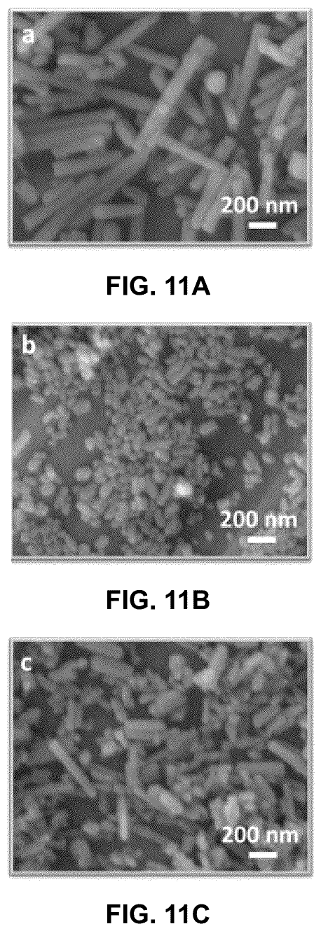

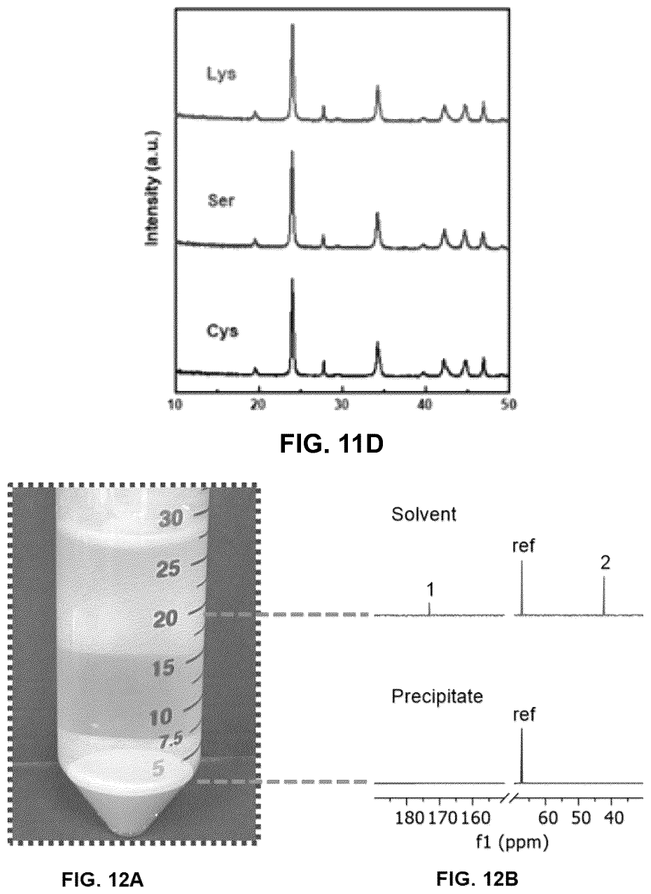

Various amino acids, including Cys, Ser, Lys, Gln, and Arg, were tested as replacements for Gly in the CO₂ capture and nanoparticle production process. Results in FIGS. 11A-11D demonstrated that these amino acids were effective in generating nanoparticles, with specific variations in size and morphology depending on the amino acid used. Specifically, the particles from Cys (FIG. 11A) exhibited a uniform diameter ranging from 100 nm to 150 nm, with a diameter-to-length ratio varying from 1:1 to 1:6. Particles derived from Ser (FIG. 11B) maintained a consistent diameter between 50 nm and 100 nm, and a diameter-to-length ratio from 1:1 to 1:2. Meanwhile, particles from Lys (FIG. 11C) varied in diameter from 50 nm to 150 nm, with the diameter-to-length ratio ranging from 1:1 to 1:6. FIG. 11D revealed that regardless of the amino acid used, the XRD spectra remained consistent, confirming that all precipitates were pure BaCO₃ and the crystalline phase remained unchanged.

### Flue Gas was Used to Produce Carbonate Nanomaterials

Table 1 shows concentration of CO₂ in flue gas from various sources.

TABLE 1
CO₂ in Flue Gas Sources

| Source | CO₂ Concentration (%) |
|---|---|
| Coal-fired power plants | 12-15 |
| Natural gas-fired power plants | 3-6 |
| Oil-fired power plants | 8-12 |
| Refineries | 8-12 |
| Industrial boilers | 5-15 |
| Petrochemical industry | 5-10 |
| Cement production | 14-33 |
| Steel production | 20-25 |
| Waste incineration | 7-10 |
| Biomass combustion | 10-20 |

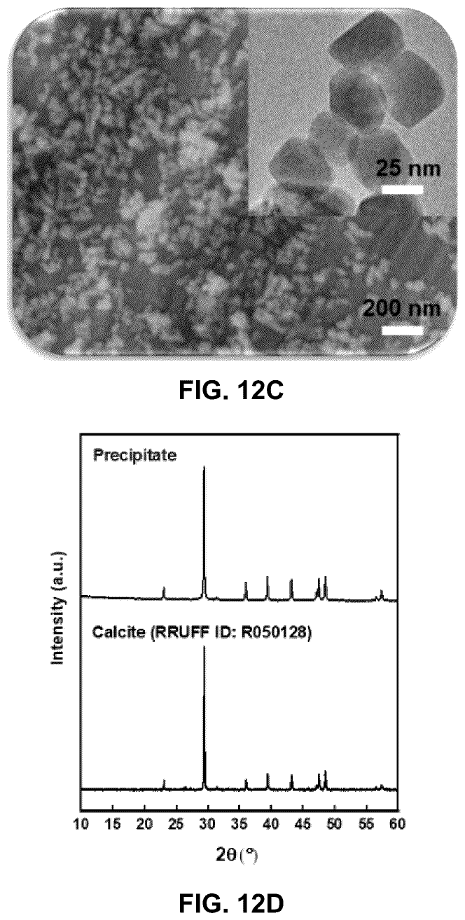

CaCO₃ nanomaterials were produced from simulated flue gases with a wide range of CO₂ levels: CO₂ capture and mineralization experiments were carried out and characterized, with the results presented in FIGS. 12A-12D. Initially, 0.025 mol Gly and 0.025 mol NaOH were dissolved in 25 mL pure water, which served as solvent. Simulated flue gas (SFG), composed of 12% CO₂ (24 sccm) and 88% N₂ (176 sccm), was then bubbled through this solution for 30 min at a flow rate of 200 sccm. Subsequently, 0.0125 mol of CaCl₂) was introduced. Following a 24-hour settling period (FIG. 12A), ¹³C NMR analysis was performed on both the supernatant and the precipitates to determine the presence of carbon-containing species (FIG. 12B). The NMR spectra revealed the presence of Gly in the solvent through peaks (1 and 2), with no detectable carbon-containing organic compounds in the precipitates (FIG. 12B). Examination by SEM and TEM (FIG. 12C) confirmed the formation of uniformly distributed nanoparticles, approximately 30 nm in size. XRD analysis verified that these nanoparticles were composed of pure calcite which was the most stable phase of calcium carbonate (FIG. 12D). This comprehensive characterization underscored the successful synthesis of calcite nanoparticles with potential implications for CO₂ sequestration technology.

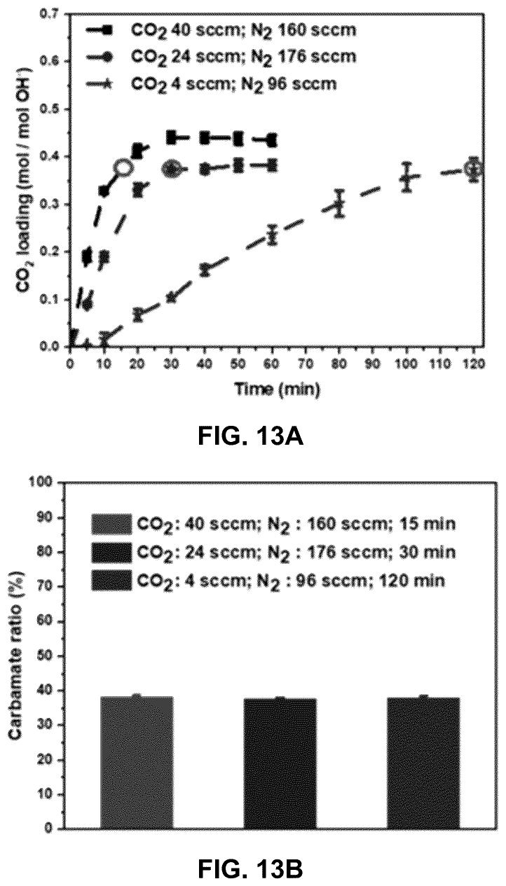

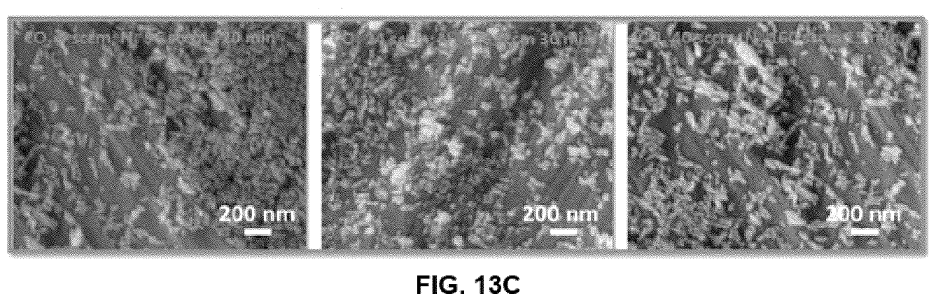

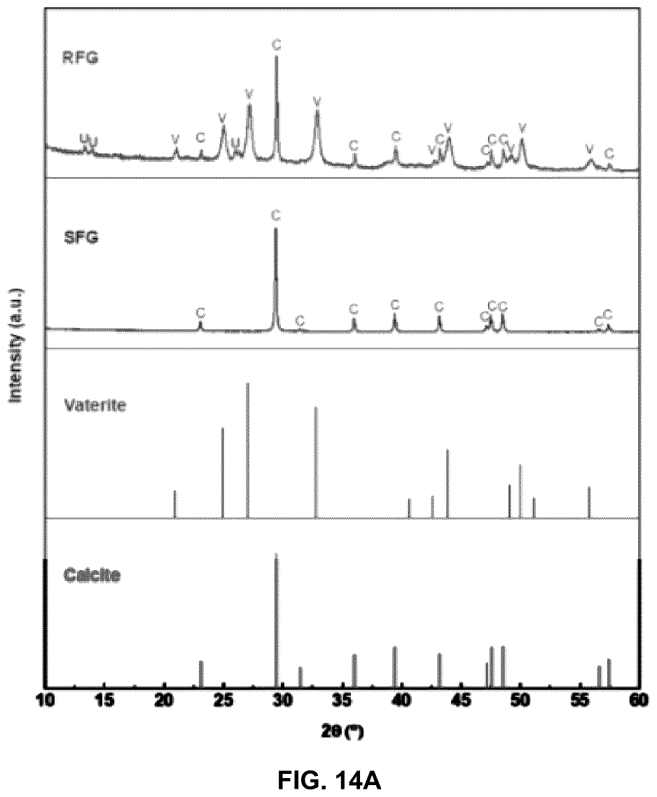

CaCO₃ nanomaterials were produced using simulated flue gas (SFG, mixing CO₂ and nitrogen) with different CO₂ concentrations (4%, 12%, 20%). It was found that CaCO₃ nanoparticles with similar morphology were obtained using SFG of different CO₂ concentrations (FIG. 13C). These experiments indicated that the disclosed method was able to convert CO₂ from SFG of different CO₂ concentrations to CaCO₃ nanomaterials.

CaCO₃ Nanomaterials were Produced from Real Flue Gas from a Power Plant

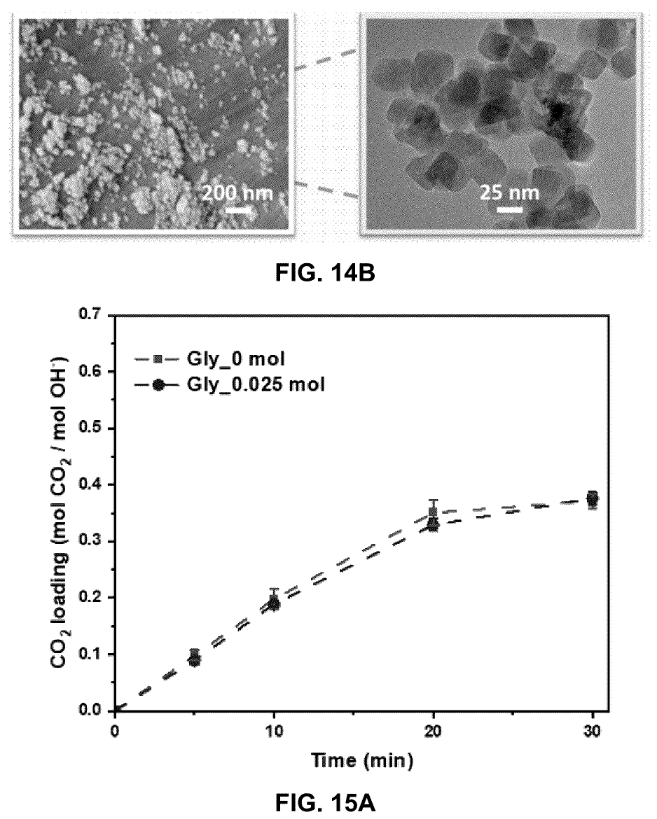

Real flue gas (RFG) was sourced from the Longview Power Plant located in Morgantown, WV, with its composition detailed in Table 2. It was used as a CO₂ source to produce CaCO₃ nanomaterials. This process resulted in the formation of nanoparticles with an average size of approximately 25 nm (FIG. 14B). XRD analysis (FIG. 14A) revealed that while CaCO₃ was the predominant chemical component, calcite was not the sole phase. Vaterite, another phase of CaCO₃, was also detected. Moreover, some unidentified peaks in the XRD spectra suggested the presence of impurities, the exact nature of which remains undetermined. These findings suggested that the presence of additional chemicals in RFG insignificantly affects nanoparticle morphology but alters the phase of CaCO₃.

TABLE 2
Composition of RFG
Pressure (atm) 1
Temperature (K) 324.82

| Component | Mol % |
|---|---|
| CO₂ | 12.022 |
| CO | 3.144 × 10⁻³ |
| O₂ | 4.79 |
| N₂ | 79.198 |
| H₂O | 0.03 |
| Ar | 0.98 |
| SO₂ | 3.319 × 10⁻³ |
| NO₂ | 3.547 × 10⁻³ |

Production of Ag₂CO₃ from Simulated and Real Flue Gas

Synthesis of nano-Ag₂CO₃ using RFG: RFG will be used as a CO₂ source to produce nano-Ag₂CO₃ based on the optimized parameters (amino acid species and amount) using SFG.

Characterization of nano-Ag₂CO₃: The solid precipitates, after being vacuum-dried, will be distributed onto a stainless-steel disc which will be subsequently adhered to SEM Specimen Stubs-Short Pin. The samples on stainless-steel discs will be coated with gold using a Denton Desk V Sputter Coater for 240 sec at an 18-mA current in an atmosphere of nitrogen. The samples will be analyzed using a JEOL JSM-7600F SEM. TEM will also be used to visualize the precipitates using a JEOL JEM-2100 TEM at an operating voltage of 120 kV; samples will be prepared by dissolving the precipitates in ethanol and then depositing onto a copper grid. ImageJ will be employed to analyze the sizes and size distribution of the particles using the SEM and TEM images. XRD analysis of the precipitates will carried out using a PANalytical X'Pert Pro diffractometer, equipped with a copper Kα radiation source. The operational settings will be maintained at 40 kV and 25 mA and utilize a LynxEye detector for enhanced sensitivity and resolution. Diffraction patterns will be systematically recorded over a 2θ angle range from 5 to 80 degrees.

Shape and size of Ag₂CO₃ can be tuned by adjusting amount and type of amino acids.

### Example 3: Antibacterial Properties of Ag₂CO₃ Nanoparticles

Nanomaterials offer several unique advantages due to their versatile composition and the ability to precisely control their physicochemical properties like size, surface chemistry, and shape. The tailored properties may enable nanomaterials to exhibit intrinsic antibacterial activities, which include causing physical damage to bacterial cell membranes, inducing nutrient starvation, and promoting light-assisted antibacterial actions and immunomodulation. It is hypothesized that nano-Ag₂CO₃ will cause the formation of free radicals (e.g., reactive oxygen species), which will adhere to bacteria cell membranes, inducing their destabilization.

The antibacterial properties of nano-Ag₂CO₃ can be evaluated using both Gram-positive and Gram-negative bacteria by measuring the minimum inhibitory concentration (MIC) and minimum bactericidal concentration (MBC). Given concerns regarding nanotoxicity, an MTT assay will also be performed to assess cytotoxicity. Nano-Ag₂CO₃ produced by RFG will be used.

Testing the antibacterial properties of nano-Ag₂CO₃: To evaluate the antibacterial properties of nano-Ag₂CO₃, MRSA (Mu50) and E. coli (DH5α) will be used as representative Gram-positive and Gram-negative bacteria, respectively. The MIC and MBC will be measured as follows:

The MIC will be determined using a broth microdilution method, where an inoculum of MRSA and E. coli will be incubated with brain heart infusion (BHI) broth at 37° C. for 18 hours while shaking at 200 revolutions per min (rpm). This inoculum will be diluted to achieve a final concentration of 1×10⁵ colony forming unit (CFU) for antibacterial studies. A stock solution of Ag₂CO₃ will be prepared and serially diluted with buffered saline (PBS) to obtain a range of concentrations (2, 4, 8, 16, and 32 μg/mL) in a 96-well plate. An equal volume of bacterial inoculum will then be introduced into the wells containing Ag₂CO₃. The plates will be incubated for 20 hours at 37° C. while shaking at 200 rpm. The MIC will be determined by examining the wells for the lowest concentration of Ag₂CO₃ that completely inhibits bacterial growth, indicated by an OD₆₀₀ reading similar to the blank (no visible growth).

Following the MIC determination, the MBC will be determined by subculturing 100 μL of the solutions that displayed no visible growth onto blood agar plates. The plates will be inverted and incubated for 24 hours at 37° C., where CFU/mL will be obtained. The MBC is determined by the lowest concentration of Ag₂CO₃ at which there is no growth observed on the blood agar plates.

The bacterial survival rate will be evaluated using the following equation:

$$Bacterial survival rate ( \% ) = N / C \times 100$$

where N is the number of colonies on the group with nano-Ag₂CO₃, and C is the number of colonies on the control group (no nanoparticles). By following these steps, the antibacterial properties of nano-Ag₂CO₃ can be systematically studied, providing insights into its effectiveness against Gram-positive and Gram-negative bacteria.

Evaluate nanotoxicity of nano-Ag₂CO₃: This experiment aims to evaluate the effects of nano-Ag₂CO₃ on the viability of human osteoblast cells under conditions where it is used as an antibacterial agent. Human osteoblast cells will be cultured in a 1:1 Ham's F12/DMEM medium supplemented with 10% fetal bovine serum (FBS), 1% penicillin-streptomycin, and 0.3 mg/ml G418 at 37° C. and 5% CO₂, with the medium refreshed every two days. Osteoblast cells will be seeded at a density of 5000 cells per well in a tissue culture-treated 96-well plate. The cells will be incubated at 37° C. and 5% CO₂ for 24-48 hours to allow adhesion and 70% confluency before treatment with nano-Ag₂CO₃. Various concentrations of nano-Ag₂CO₃ (2, 4, 8, 16, and 32 μg/mL) will be added to the wells with growth medium without phenol red and incubated for 24 hours at 37° C. Fresh medium without nano-Ag₂CO₃ will serve as the negative control. After incubation with nano-Ag₂CO₃, 10 μL of MTT solution will be added to the wells containing 100 μL of media without phenol red. This will be incubated for 4 hours at 37° C. to allow for the formation of formazan crystals. Finally, 150 μL of DMSO will be added to the wells to dissolve the crystals and the absorbance of the wells will be recorded at 570 nm.

The cell viability percentage will be calculated by comparing the absorbance of treated samples with that of the control group. All experiments will be performed in triplicate, and the average values will be reported.

By following these steps, the nanotoxicity of nano-Ag₂CO₃ on human osteoblast cells can be evaluated, providing insights into its safety and efficacy as an antibacterial agent.

Characterization of bacteria: SEM will be utilized to observe the changes after nano-Ag₂CO₃ treatment. 1 mL of bacterial suspensions will be treated with 1 mL of nano-Ag₂CO₃ aqueous suspension and incubated with shaking at 37° C. for 2 hours. The mixture will then be separated by centrifugation at 3600 rpm for 3 min, rinsed with PBS buffer, and fixed with 4% glutaraldehyde for 4 hours at 4° C. The bacterial samples will be rinsed with PBS again, followed by dehydration using sequential ethanol treatments (each step lasting 12 min), and then dried for further characterization. Bacteria without nano-Ag₂CO₃ treatment will serve as the control group.

Effectiveness of Nano-Ag₂CO₃ for Wound Recovery

CO₂ can promote angiogenesis, this mechanism enhances the formation of new blood vessels, improving blood supply to damaged tissues and promoting wound healing. CO₂ activates cellular metabolic activities, increasing cell proliferation and protein synthesis. This is crucial for the functional recovery of key cells, such as fibroblasts, aiding in wound healing. Angiogenesis is a prerequisite for morphological and functional healing, leading to the rebuilding of damaged vessels, re-establishment of blood flow, and restoration of the O₂ supply to tissues. Human umbilical vein endothelial cells (HUVECs), which are widely used for in vitro angiogenesis, will be used to study the potential effectiveness of nano-Ag₂CO₃ for angiogenesis. It is hypothesized that CO₂ will be released from Ag₂CO₃ in simulated biological fluids, and that the CO₂ released from Ag₂CO₃ will stimulate angiogenesis.

Experimental Design: HUVECs will be cultured and assessed for proliferation and tube formation before and after nano-Ag₂CO₃ treatment. Techniques including AlamarBlue for proliferation and matrigel assays for tube formation will be used for comprehensive evaluation of cell behavior.

HUVEC Culture: HUVECs will be isolated using 0.1% type I collagenase and cultured in complete endothelial cell medium (ECM) supplemented with 5% fetal bovine serum (FBS), endothelial cell growth supplement, and penicillin/streptomycin at 37° C. in a 5% CO₂ incubator. The medium will be changed every two days until the cells reach 80-90% confluence. Third-passage (P3) HUVECs will be used for the experiments. HUVECs will be seeded at a density of 30,000 cells/50 μL in media containing different concentrations of nano-Ag₂CO₃ solutions (2, 4, 8, 16, 32 μg/mL) and incubated for one hour at 37° C. in a 5% CO₂ atmosphere. Non-adherent cells will be gently washed off with PBS, and the cells will be continuously cultured in media containing the respective concentrations of nano-Ag₂CO₃.

Cell Proliferation Assessment: Cell proliferation will be assessed using the AlamarBlue assay. HUVECs will be cultured in media containing nano-Ag₂CO₃ solutions at concentrations of 2, 4, 8, 16, and 32 μg/mL. After the specified incubation periods, the cells will be incubated with 10% AlamarBlue reagent at 37° C. in a 5% CO₂ atmosphere for four hours. The optical density (OD) will be measured at 570 nm with a reference wavelength of 600 nm using a microplate reader to determine the cell proliferation rate.

Matrigel tube formation assay: A matrigel tube formation assay will be conducted to evaluate the effects of nano-Ag₂CO₃ on HUVEC angiogenesis. Tissue culture dishes will be pre-coated with a thin layer of 2% agarose gel, creating three holes. Nano-silver carbonate solutions at different concentrations will be mixed with media and 1×10⁵ HUVECs in 80 μL of medium at a 1:1 ratio with Matrigel. The mixture will be applied into the holes and incubated at 37° C. for 15 min to allow the Matrigel to gel. Medium will be added, and the plates will be incubated at 37° C. in a 5% CO₂ atmosphere for seven days, with the medium changed every three days. On day seven, the Matrigel will be removed from the samples and placed on a glass slide for examination of capillary-like tube formation under a light microscope, and images will be captured using a digital camera.

### Example 4: Conversion of CO₂ from Power Plants into CaCO₃ Nanoparticles

### Materials and Methods

Materials: Gly (≥98 wt %), NaOH (≥98 wt %), and CaCl₂) (≥97 wt %) were procured from Sigma-Aldrich for the purpose of CO₂ capture and mineralization. In addition, deuterium oxide (D₂O, ≥99.9 wt %) and 1,4-dioxane (≥99.5 wt %) were also purchased from the Sigma-Aldrich for nuclear magnetic resonance (¹³C NMR) analysis. Research grade CO₂ and ultra-high pure N₂ were supplied by Airgas.

BEAS-2B cells were obtained from the National Institute for Occupational Safety and Health (NIOSH) and maintained according to standard cell culture protocols, similar to previous studies. Fetal bovine serum (FBS), penicillin-streptomycin solution (100×), and Dulbecco's Modification Eagles Medium (DMEM) containing 14 g/L, L-glutamine, and sodium pyruvate, and BD-Falcon tissue culture plates, 96 well, flat bottom with low evaporation lid were purchased from Corning for BEAS-2B cell culture. Dulbecco's Phosphate Buffered Saline (PBS) without calcium chloride and magnesium chloride, as well as 0.25% Trypsin-EDTA (1×), were purchased from Gibco. The CyQUANT MTT Cell Viability Assay Kit was purchased from Thermo Fisher Scientific.

CO₂ absorption and mineralization: In this study, CO₂ absorption and conversion were studied in laboratory using real flue gas (RFG) collected from Longview Power Plant (Maidsville, WV). Major constituent data in RFG were supplied by Longview Power Plant personnel. Simulated flue gases (SFG) produced by mixing CO₂ and N₂ at different ratios were also studied and used to optimize the nanoparticle formation.

For CO₂ absorption and conversion, NaOH (0.025 mol), a specific amount of Gly (0 and 0,025 mol), and 25 ml of water were mixed in a centrifuge tube as the solvent for CO₂ absorption. A serological pipette was placed at the bottom of tube, acting as an outlet for flue gas. CaCO₃ nanoparticles were prepared in a two-step process: In the first step, CO₂ from RFG or SFG was absorbed using the Gly solvent prepared, and then in a second step, a Ca²⁺ salt was added to form nanoparticles. The concentration and flow rate of flue gas were controlled by mass flow controllers. Once the solvent was bubbled with flue gas for a predetermined duration, 0.0125 mol CaCl₂) was added to the solvent and the sample was vortexed for 1 min. After storage for 24 h, the tubes were subjected to centrifugation at 4,000 rpm for five min to isolate the precipitates. The precipitates were washed and subjected to repeated centrifugation five times. The amount of captured CO₂ was quantified by tracking the mass of the solution at different time points during the CO₂ bubbling process. The dry weight of the precipitates was determined after subjecting it to vacuum drying for 72 h at room temperature and employing an analytical balance that offers a precision up to 0.0001 g.

Because the dosage of NaOH (0.025 mol OH⁻) was identical under the various conditions, the CO₂ loading and CaCO₃ weight were evaluated as shown below:

$${CO}_{2} loading ( mol / mol {OH}^{-} ) = captured {CO}_{2} / 0.025 \qquad ( 1 )$$

$${CaCO}_{3} weight ( mol / mol {OH}^{-} ) = produced {CaCO}_{3} / 0.025 \qquad ( 2 )$$

### Characterization

¹³C Nuclear magnetic resonance (¹³C NMR): A 400 MHz Agilent NMR instrument was utilized to identify carbon species in solvents. Initially, a mixture comprising 500 μL of sample solution, 200 μL of D₂O, and 15 μL of 1,4-dioxane (as the internal standard) was prepared in a centrifuge tube and thoroughly mixed. This homogeneous solution was then transferred to an NMR tube for ¹³C NMR spectral analysis, conducted with a delay time of 1 sec and 256 scans. To determine the presence of Gly or carbamates in the precipitates, 10 mg of the precipitates were rinsed in a mixture of 700 μL D₂O and 15 μL 1,4-dioxane, and the supernatant was subsequently analyzed via NMR.

There were only five carbon-containing chemicals in the system, and their chemical shifts were numerically labelled (Scheme 1). Peak (6) in the NMR spectra, shifting between 161 ppm and 168 ppm, corresponds to CO₃²⁻/HCO₃⁻. The presence of carbamate was confirmed through its characteristic peaks at 46 ppm (peak 4, CH₂), 165 ppm (peak 5, C═O), and 180 ppm (peak 3, C═O). In addition, peaks representing Gly/Gly⁻ might shift from 182 to 174 ppm (peak 1, C═O) and from 45 to 42 ppm (peak 2, CH₂). The reference peak for 1,4-dioxane at 67.19 ppm was used for calibration but was not included in the final analysis to maintain focus on the targeted solutes. The identification of each carbon signal was numerically annotated for clarity.

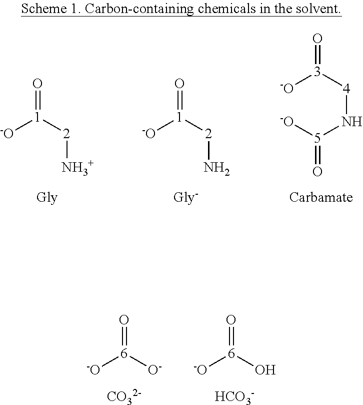

Given the molecular structure of carbon species involved in Scheme 1, Gly can only undergo conversion to carbamate. Therefore, the initial molar quantity of Gly/Gly⁻ should match the combined molar amounts of Gly/Gly⁻ and carbamate at any given time point. In this study, peaks 2 and 4 were assigned to Gly/Gly⁻ and carbamate, respectively. The variations in the areas of these peaks served to quantify the concentration changes of both carbamate and Gly/Gly⁻ in the solvent. This relationship was quantitatively expressed and analyzed using the following equations.

$$Carbamate ratio ( \% ) = ( area of peak 4 ) / ( area of peak 2 + area of peak 4 ) \times 100 \% \qquad ( 3 )$$

$$Gly / {Gly}^{-} percentage ( \% ) = 100 - Carbamate percentage \qquad ( 4 )$$

Electron microscopy: The preparation of the dry precipitates involved evenly spreading the precipitates on a stainless-steel disc, which was then fixed to a pin. This setup underwent a coating process in a Denton Desk V Sputter Coater for 240 sec at 18 mA, under a nitrogen atmosphere to avoid oxidation. The samples were examined with a JEOL JSM-7600F scanning electron microscope (SEM), capturing images at 5 kV acceleration voltage and about 15 mm working distance. To analyze the particle sizes in more detail, transmission electron microscopy (TEM) was employed. For TEM sample preparation, the precipitates were suspended in ethanol, forming a dilute suspension that was spread on a copper grid for imaging. The TEM imaging was conducted at an operational voltage of 120 kV.

X-ray diffraction (XRD): XRD assessment of the dried precipitates employed a PANalytical X'Pert Pro diffractometer, featuring a copper Kα radiation source. It operated at 40 kV and 25 mA, with a LynxEye detector. Diffraction patterns were consistently captured across a 2θ angle ranging from 5 to 80 degrees.

### BEAS-2B Cell Viability Evaluation

Particle preparation and cell culture conditions: CaCO₃ nanoparticles produced by RFG and SFG using a CO₂ concentration of 12% were evaluated for their toxicity. For cell viability experiments, CaCO₃ nanoparticle suspensions were prepared in sterile PBS at a stock concentration of 4 mg/mL. Nanoparticles in the stock were sonicated in a water bath for 30 sec to ensure particle dispersion. Prior to cytotoxicity experiments, the stock nanoparticle suspension was diluted to concentrations of 10 to 400 μg/mL using DMEM supplemented with 10% FBS. BEAS-2B cells were cultured in a T75 tissue culture flask using DMEM with 1 g/L glucose, 4 mM L-glutamine, 1 mM sodium pyruvate, 10% FBS, and 1% penicillin/streptomycin. The culture medium was replaced every other day and maintained in an incubator at in a humid atmosphere at 37° C. and 5% CO₂ air. When BEAS-2B cells reached 80% confluency, they were detached using trypsin, neutralized with DMEM containing FBS, transferred to a sterile 15 mL centrifuge tube, and centrifuged at 1200 rpm for 7 min at 4° C. to form a cell pellet.

Cell viability assay: Cells were suspended in DMEM and plated at a density of 1.0×10⁴ cells/well in a 96-well plate and incubated for 24 h to allow for cell adherence. Cells were exposed to 100 μL CaCO₃ nanoparticles at concentrations of 10, 100, and 400 μg/mL for exposure periods of 6, 18, and 24 h with three replicates per condition. A control group was treated with 100 μL of medium without nanoparticles under the same conditions. Following treatment, the media with the nanoparticles were removed and replaced with 100 μL of fresh DMEM medium without phenol-red. MTT cell viability assay was performed according to the CyQUANT MTT Cell Viability Assay Kit instruction, using MTT reagent and SDS-HCl as a solubilizing agent. After incubating with the solubilizing agent, the absorbance of each well was measured at 570 nm. The blank wells containing only media, MTT, and SDS-HCl (no cells) were subtracted from the absorbance readings for each well. Cell viability was determined by dividing the absorbance of the cells incubated with the particles by the absorbance of control cells containing only media.

Statistical analysis: Analysis of variance (ANOVA) was conducted, followed by Tukey's HSD test for multiple comparisons, using JMP Pro 18 software. Prior to analysis, key assumptions of ANOVA were assessed: normality of residuals was confirmed using the Quantile-Quantile plot, and homogeneity of variances was validated with Levene's test. A p-value of less than 0.05 was considered to indicate a statistically significant difference.

### Results

Capturing and converting CO₂ from RFG into CaCO₃ nanoparticles: Typically, flue gases emitted from fossil fuel-based power plants contain not only CO₂ and N₂ but also minor quantities of NOₓ, SOₓ, and particulate matter. In this study, flue gas, sourced from Longview Power Plant (Maidsville, WV), was studied; the composition of the flue gas was detailed in Table 2. Notably, the flue gas had about 12% CO₂ and 79% N₂, and some additional gases such as O₂, CO, SO₂, etc. In this study, the flue gas was bubbled at a flow rate of 200 standard cubic centimeters per minute (sccm) for 30 min through the solvent that was prepared by dissolving 0.025 mol NaOH and 0.025 mol Gly in 25 mL of pure water. This process resulted in the formation of solid precipitates. Interestingly, XRD analysis (FIG. 14A) revealed that while CaCO₃ was the predominant chemical component, calcite was not the sole phase; vaterite, another phase of CaCO₃, was also detected. Moreover, some peaks in the XRD spectra were not able to be identified, suggesting the capture and conversion of impurities into insoluble particles, the exact nature of which remains unknown. It is unknown if the small amounts of reactive gas components like SO₂ might have led to the formation of precipitates and future studies may look into this aspect. Electron microscope images showed that the precipitates were nanoparticles with an average size of approximately 25 nm (FIG. 14B).

Capturing and converting CO₂ from SFG into CaCO₃ nanoparticles: CO₂ capture and mineralization experiments using SFG were carried out and the results were presented in FIGS. 12A-12D. Initially, 0.025 mol of Gly and 0.025 mol of NaOH were dissolved in 25 mL of pure water and served as the solvent. SFG, composed of 12% CO₂ (24 sccm) and 88% N₂ (176 sccm), was then bubbled through this solvent for 30 min at a flow rate of 200 sccm. Next, 0.0125 mol of CaCl₂) was introduced to mineralize the captured CO₂. Following a 24-h settling period (FIG. 12A), ¹³C NMR analysis was performed on both the supernatant and the precipitates to determine the presence of carbon-containing species (FIG. 12B). The NMR spectra revealed the presence of Gly/Gly⁻ in the solvent through peaks (1 and 2), with no detectable carbon-containing organic compounds in the precipitates (FIG. 12B). Moreover, NMR data showed that carbamate (represented by peaks 3, 4, and 5) was clearly formed at 10 min and CO₃²⁻/HCO₃⁻ (represented by peak 6) appeared at 30 min. Examination by SEM and TEM (FIG. 12C) confirmed the formation of nanoparticles, approximately 25 nm in size. XRD analysis found that these nanoparticles were composed of pure calcite which was the most stable phase of calcium carbonate (FIG. 12D).

### Factors Influencing CaCO₃ Nanoparticle Production

Role of Gly on CaCO₃ formation: To determine the role of Gly in CO₂ capture and nanomaterials production when flue gas was used as the CO₂ source, comparative experiments were conducted with and without Gly. First, solvent was prepared by dissolving 0.025 mol NaOH in 25 mL of pure water with 0 mol or 0.025 mol of Gly. Then SFG (CO₂: 12%; N₂: 88%) was bubbled through the solvent for 30 min at a flow rate of 200 sccm. After that, 0.0125 mol of CaCl₂) was added to form CaCO₃ precipitates.

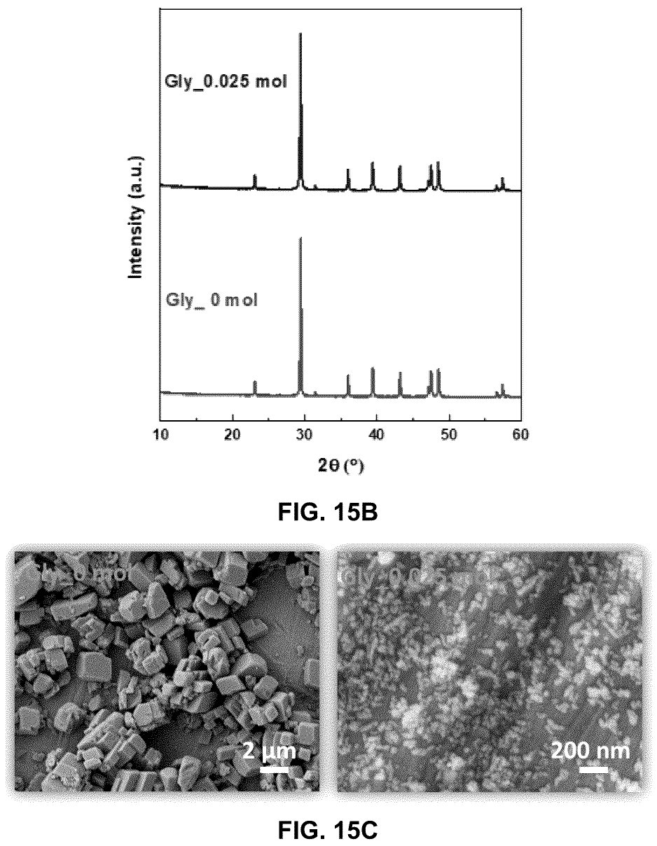

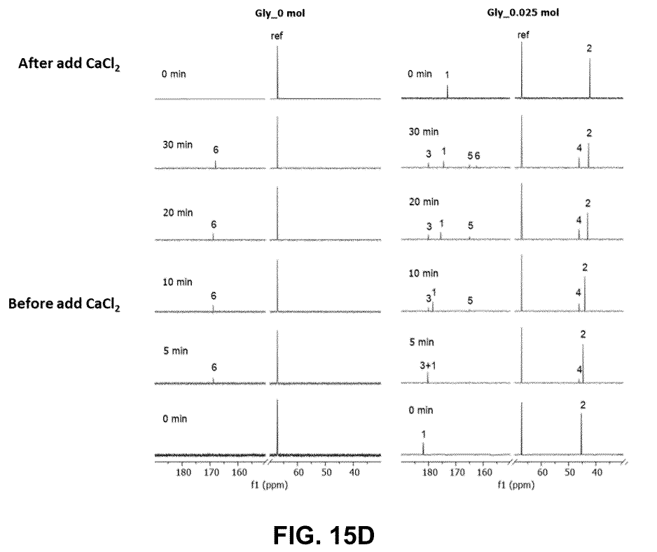

As illustrated in FIG. 15A, the CO₂ absorption curves were similar with and without Gly, suggesting that the addition of Gly did not impact CO₂ loading. XRD (FIG. 15B) confirmed that Gly did not influence the type of phases and the sole crystalline phase calcite was produced in both the presence and absence of Gly. However, the SEM images (FIG. 15C) revealed a stark contrast in particle size between the two solvents. Without Gly, the particles averaged around 1-2 μm, making them roughly 50 times larger than those obtained from the Gly-containing solvent, where particles were averagely 25 nm. This suggested that Gly played a critical role in deciding the size of the CaCO₃ particles.

Analyzing the NMR spectra (FIG. 15D) offered insights into the different reaction pathways. In the absence of Gly, no peaks appeared prior to CO₂ bubbling. Upon CO₂ bubbling, peak 6 associated with CO₃²⁻/HCO₃⁻ emerged, shifting from 5 min (168.89 ppm) to 30 min (168.11 ppm), and subsequently disappeared after the addition of CaCl₂), indicating full consumption into CaCO₃. Conversely, the solvent containing Gly/Gly⁻ showed peaks 1 and 2 before CO₂ bubbling; the introduction of CO₂ led to the gradual appearance of peaks 3, 4, and 5, which correspond to carbamate, alongside peak 6 for CO₃²⁻/HCO₃⁻ at the 30-min mark. After CaCl₂) addition, all peaks disappeared except for those (peaks 1 and 2) representing Gly/Gly⁻, indicating the complete consumption of carbamate and CO₃²⁻/HCO₃⁻ to form CaCO₃ and left only Gly/Gly⁻ in the solution.

Effects of time when CaCl₂) was added on CaCO₃ formation: Solvent was prepared by dissolving 0.025 mol NaOH and 0.025 mol Gly in 25 mL pure water. This solvent was then bubbled with SFG (12% CO₂, 88% N₂) at a flow rate of 200 sccm for varying durations, after which 0.0125 mol of CaCl₂) was introduced to form CaCO₃ precipitates.

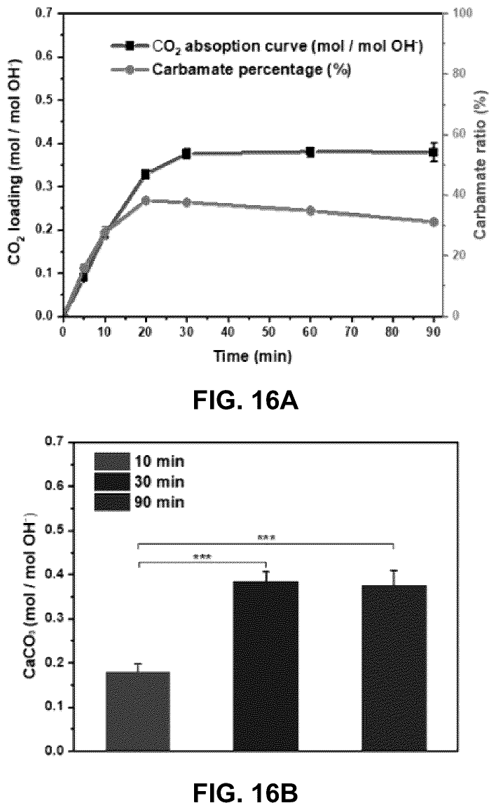

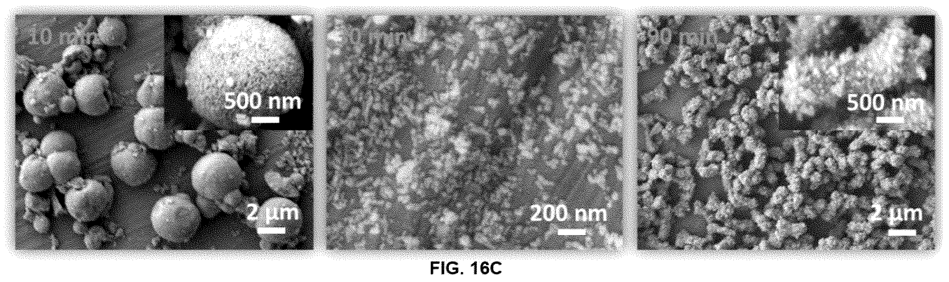

FIG. 16A displayed the temporal dynamics of CO₂ capture, showing that maximum loading, approximately 0.38 mol CO₂/mol OH⁻, was obtained after 30 min bubbling. Prolonging the bubbling time beyond 30 min did not change the CO₂ loading. NMR data (FIG. 16A) from 0 to 30 min confirmed an initial increase in carbamate concentrations, which then decreased from 30 min (~38%) to 90 min (~31%). The molar mass of CaCO₃, derived from the OH⁻ dosage, showed yields of about 0.18 mol CaCO₃/(mol OH) at 10 min, which increased to 0.38 mol CaCO₃/(mol OH⁻) at both 30 and 90 min intervals (FIG. 16B). This molar mass of CaCO₃ aligned with the moles of captured CO₂. The size and shape of CaCO₃, influenced by the varying durations of CO₂ bubbling, were presented in FIG. 16C. At 10 min, porous balls of various sizes appeared, while at 30 min, nanoparticles (roughly 25 nm in size) were observed. At 90 min, uniformly structured rods, approximately 800 nm in diameter and 2 μm in length, were formed.

Effects of CO₂/N₂ ratio on CaCO₃ formation: To assess the versatility and effectiveness of the disclosed method in adapting to various industrial emissions (e.g. power plant, cement plant, chemical plant) with different CO₂ concentration and to reflect the potential for broader implementation in CO₂ capture strategies, SFGs with CO₂ concentrations of 4% were employed (CO₂: 8 sccm; N₂: 192 sccm), 12% (CO₂: 24 sccm; N₂: 176 sccm), and 20% (CO₂: 40 sccm; N₂: 160 sccm) as the CO₂ sources, maintaining a constant flow rate of 200 sccm. The solvent used for these experiments consisted of 0.025 mol NaOH and 0.025 mol Gly in 25 mL pure water.

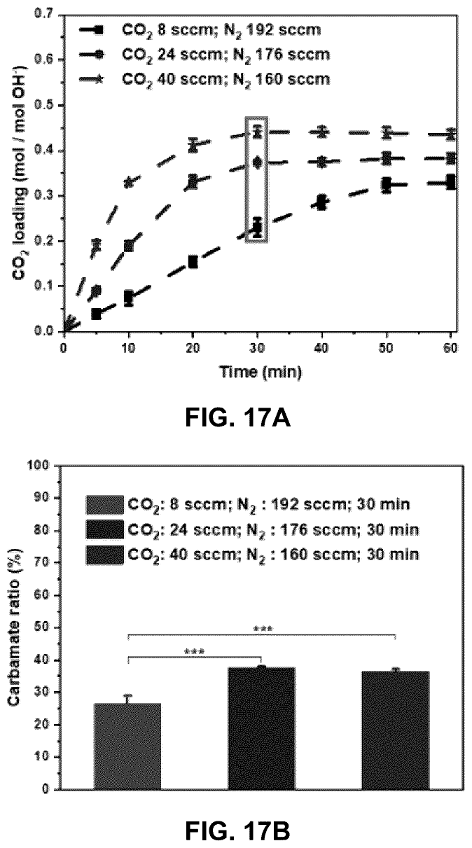

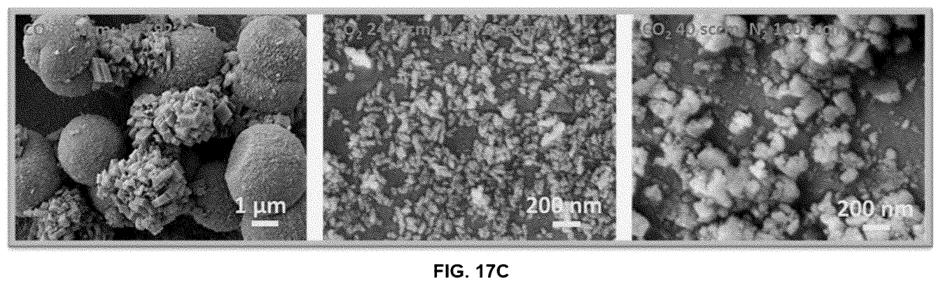

The CO₂ loading curve (FIG. 17A) distinctly illustrated that the rate of CO₂ absorption varied markedly across different CO₂ concentrations. Specifically, at a 30-min interval before the addition of CaCl₂), CO₂ loading achieved values of 0.23, 0.37, and 0.44 mol CO₂/(mol OH⁻) for CO₂ concentrations of 4%, 12%, and 20%, respectively. Furthermore, NMR spectra analysis (FIG. 17B) revealed variations in the quantity of carbamates formed, obtaining 27%, 38%, and 36% of carbamate at the respective CO₂ concentrations of 4%, 12%, and 20% right before the addition of CaCl₂) at the 30-min mark. SEM imaging (FIG. 17C) depicted the differences of size and shape of the precipitates using different CO₂ concentrations after CaCl₂) was added at 30 min. At a CO₂ concentration of 4%, the SEM showcased a mix of fluffy spherical microparticles (~3 μm) and aggregates of cubic particles (~500 nm in size). At a CO₂ concentration of 12%, nanoparticles (roughly 25 nm in size) were observed. At a CO₂ concentration of 20%, the particle sizes were noticeably uneven, displaying a wide diameter range from just a few nanometers up to approximately 200 nm. SEM imaging indicated significant size and shape changes correlating with CO₂ concentration levels.

Influence of N₂ flow rate on CaCO₃ formation: Solvent was prepared by dissolving 0.025 mol NaOH and 0.025 mol Gly in 25 mL pure water. 24 sccm of CO₂ was combined with varying N₂ flow rates of 100 sccm, 176 sccm, and 250 sccm through the solvent for 30 min.

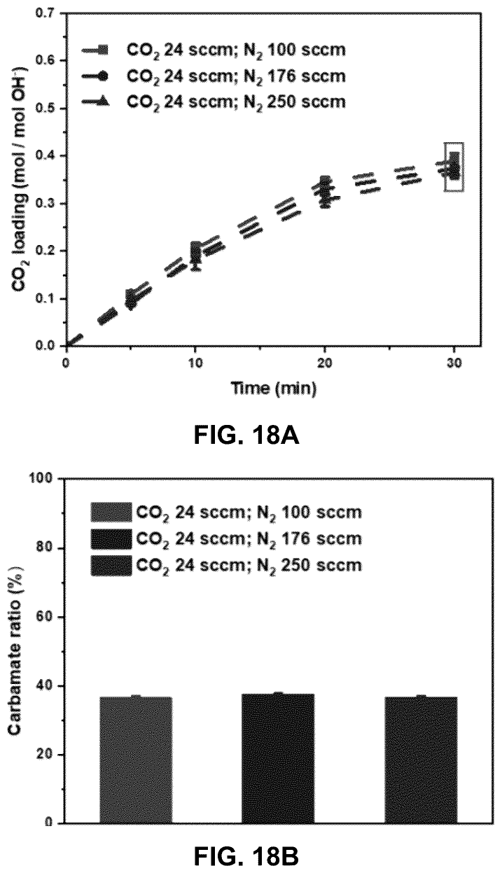

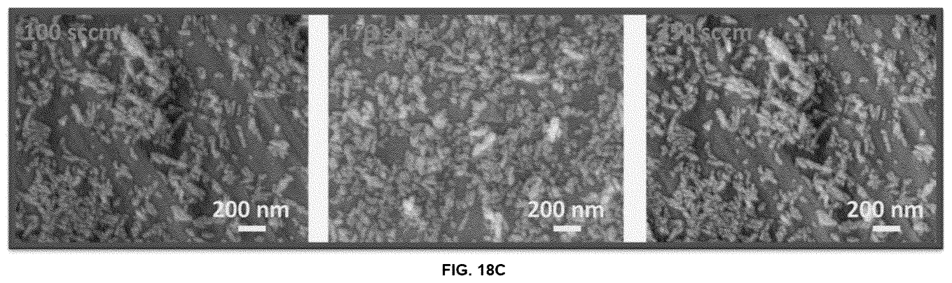

As observed in FIG. 18A, the CO₂ absorption did not undergo significant changes. NMR data indicated that the carbamate levels (FIG. 18B) remained almost constant at the 30-min mark. Upon analyzing the particle size and shape (FIG. 18C), particles of nearly identical size and shape were formed after the addition of CaCl₂) at 30 min. These findings suggested that the presence of N₂, when the CO₂ flow rate was fixed at 24 sccm, did not have significant effects on the CO₂ loading and the size and shape of the resulting particles.

Producing similar CaCO₃ nanoparticles using SFGs with different CO₂ concentrations: Methods to synthesize nanoparticles of similar sizes and shapes from SFGs with varying CO₂ concentrations were sought. Solvents were prepared containing 0.025 mol NaOH and 0.025 mol Gly and bubbled them with SFGs; different CO₂ ratios, flow rates, and bubbling time were applied to achieve the same CO₂ loading and carbamate percentages. For the CO₂ concentration of 20%, the bubbling time was decreased from 30 min to 15 min. Just before the addition of CaCl₂), the CO₂ loading (FIG. 13A) and carbamate percentage (FIG. 13B) were about the same as those observed with a 12% CO₂ concentration at 30 min. For the CO₂ concentration of 4%, the SFG flow rate was reduced from 200 sccm to 100 sccm and extended the bubbling time to 120 min, which led to comparable CO₂ loading (FIG. 13A) and carbamate levels (FIG. 13B) with those of 12% CO₂ concentration at 30 min. SEM images confirmed that similar CaCO₃ nanoparticle sizes and shapes were obtained in the three different (i.e., 4%, 12,%, and 20%) CO₂ levels (FIG. 13C).

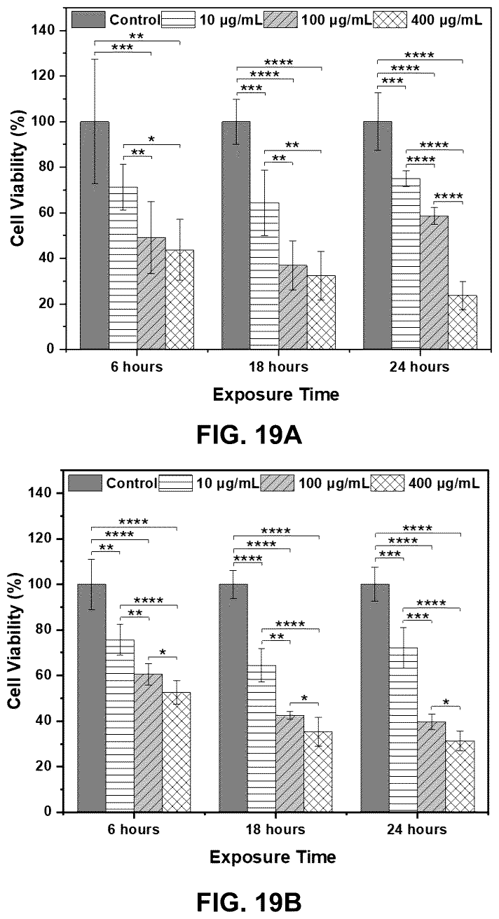

Toxicity of CaCO₃ nanoparticles produced from RFG and SFG: BEAS-2B cells were exposed to CaCO₃ nanoparticles produced from RFG at various timepoints and concentrations (FIG. 19A). After 6 h exposure, the cell viability significantly decreased by 36% and 45% in the 100 and 400 μg/mL nanoparticle treated samples when compared to the control. Although 10 μg/mL was not significantly different from the control, the decrease in viability approached significance (p=0.053). There was also a significant decrease in cell viability when exposed to 100 and 400 μg/mL when compared to 10 μg/mL. The effects of exposure were also measured after 18 h, where there was a significant decrease of 15%, 46%, and 77% in 10, 100, and 400 μg/mL when compared to the control. Additionally, cell viability significantly decreased at 100 and 400 μg/mL compared to 10 μg/mL. After an exposure of 24 h, there was a significant reduction in viability at concentrations of 10, 100, and 400 μg/mL when compared to the control. The cell viability also significantly decreased at 100 and 400 μg/mL compared to 10 μg/mL, with an additional reduction observed at 400 μg/mL compared to 100 μg/mL.

BEAS-2B cells were also exposed to CaCO₃ nanoparticles obtained from SFG at various timepoints and concentrations (FIG. 19B). After 6 h of exposure to the nanoparticles, cell viability significantly decreased by 22%, 29%, and 32% at 10, 100, and 400 μg/mL compared to the control. There was also a significant decline in viability at 100 and 400 μg/mL compared to 10 μg/mL, with an additional decrease observed at 400 μg/mL compared to 100 μg/mL. A similar trend was observed after 18 and 24 h of exposure, with higher concentrations consistently leading to greater reductions in viability.

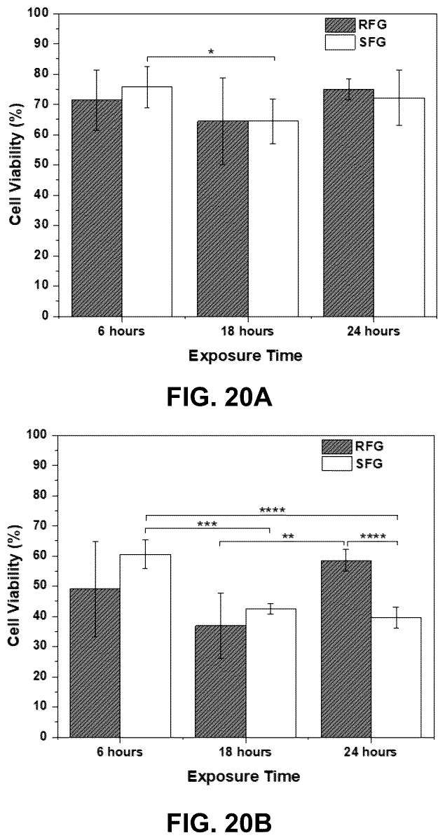

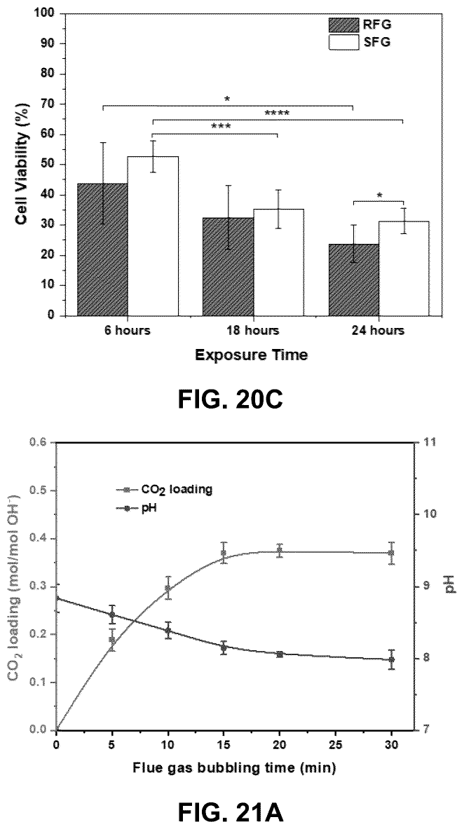

The cell viability upon exposure to CaCO₃ nanoparticles was further compared between the nanoparticle treatment time and between nanoparticles produced from RFG and SFG (FIGS. 20A-20C). When treated with 10 μg/mL CaCO₃ nanoparticles, cell viability higher than 60% was observed in all cases, and there was no significant difference in the cell viability between nanoparticles produced from RFG and SFG. However, significantly reduced cell viability was observed at 18 h compared to 6 h exposure when exposed to nanoparticles produced from SFG (FIG. 20A). When treated with 100 μg/mL CaCO₃ nanoparticles (FIG. 20B), significantly reduced cell viability was found at 18 h and 24 h when compared to 6 h treatment of nanoparticles from SFG, while significantly higher cell viability was observed at 24 h compared to 18 h for nanoparticles from RFG. There was no significant difference in cell viability between RFG and SFG at 6 h and 18 h and a significant difference was observed at 24 h exposure. At 400 μg/mL, when exposed to nanoparticles from RFG, there was a significant decrease in viability between 6 and 24 h (FIG. 20C). When comparing exposure to nanoparticles from SFG, there were significant decreases in cell viability at 18 h and 24 h when compared to 6 h exposure. There were no significant differences in cell viability for nanoparticles produced from SFG and RFG at 6 h and 18 h exposures, while a significant difference was observed at 24 h exposure.

### Discussion

CO₂ capture is crucial for reducing carbon emissions from fossil fuel combustion. To keep atmospheric CO₂ levels below 350 ppm, at least 550 gigatons of CO₂ need to be removed by the end of this century. CO₂ can be captured from sources (point source capture) or ambient air (direct air capture, DAC). Compared to point source capture, DAC is an emerging technology and currently quite costly, with expenses reaching up to $1,000/ton of CO₂ removed. This high cost is primarily due to the low concentration of CO₂ in the air, necessitating large and expensive capture units. In addition, DAC is highly energy-intensive, requiring substantial amounts of electrical or thermal energy. There are three main methods for capturing CO₂ from point source: pre-combustion, post-combustion, and oxy-fuel combustion. Pre-combustion capture involves conversion of fossil fuels to CO₂ and H₂, where CO₂ is captured before combustion while hydrogen is used as fuel. Oxy-fuel combustion burns fuel in oxygen, not air, leading to a flue gas primarily composed of CO₂ and the impurities. Post-combustion carbon capture is highly efficient for reducing carbon emissions as it requires minimal modifications to existing facilities and operates under near ambient conditions. It is particularly relevant for addressing climate change since many current emission sources, such as electricity generating units, can only work with post-combustion capture technologies.

Amine-based technology has been commercially used for post-combustion CO₂ management. However, it faces several challenges, including the toxic volatiles, corrosion, and hazardous by-products. Amino acid salt solvents are considered as a promising candidate for CO₂ management because they are more environmentally friendly and have lower evaporation and higher stability. In addition, amino acids like Gly can be recovered and reused using existing method. Carbon mineralization is thermodynamically favored, and CO₂ is permanently fixed.

In this study, amino acid salt solvents were developed for post-combustion CO₂ absorption and conversion, and CaCO₃ nanoparticles (~25 nm) were synthesized, for the first time, using flue gases from a power plant. These nanoparticles presented two phases (i.e., calcite and valerate), and this was different from those nanoparticles produced from SFGs which had only calcite. The reason for this difference is unknown and might be related to the other chemical components within the power plant flue gases; flue gases from power plants usually contain not only CO₂ and N₂ but also water, acidic gases, and some other impurity. Another difference observed was that there was a very small amount of other chemicals presented in the nanoparticle products obtained from the power plant flue gases since there were unidentified weak peaks in the XRD data. Such nanoparticles synthesized from power plant flue gases may be used in industries with relatively low requirement of impurity like construction, but they should be purified before they are used in areas like biomedical applications where high purity is required.

Moreover, the data (FIGS. 13A-13C and 15A-18C) clearly illustrated the crucial role of Gly in the production of CaCO₃ nanoparticles; without Gly, most of CaCO₃ particles were 1-2 μm, and with Gly the CaCO₃ were about 25 nm (FIGS. 15A-15D). With Gly, CO₂ reacted with deprotonated Gly to form carbamate first, and then hydrolyzed to bicarbonate. Without Gly, CO₂ directly reacted with OH to form HCO₃ (FIG. 15D). It seems that different carbamate percentages (FIG. 16A) led to different particle sizes and shapes (FIG. 16C), and different CO₂ loadings (FIG. 17A) also resulted in different particle sizes and shapes (FIG. 17C) no matter if the carbamate percentage was the same or different (FIG. 17B). However, the similar CO₂ loading (FIG. 18A) and similar carbamate percentages (FIG. 18B) led to similar particle size and shapes (FIG. 18C). Therefore, it was hypothesized that CaCO₃ particles of similar shapes and sizes could be obtained from different flue gases of different CO₂ levels if the same Gly dosage, CO₂ loading, and carbamate levels could be obtained. The present experimental findings (FIGS. 13A-13C) validated this hypothesis and confirmed that the nanoparticles were determined by factors including Gly dosage, CO₂ loading, and the amount of carbamate formed (before CaCl₂) was added). It was shown that by adjusting the bubbling time and flue gas flow rate, CO₂ loading and carbamate ratio can be adjusted to produce CaCO₃ nanoparticles using flue gases with different CO₂ concentrations (FIGS. 13A-13C). This suggested that CaCO₃ nanoparticles of similar sizes and shapes can be obtained using flue gases from various sources like coal- and natural gas-fired power plants.

In this study, CaCl₂) was used as a model chemical source for Ca²⁺. For industrial applications, CaCl₂) can be found in naturally occurring minerals like Antarcticite and Sinjarite. CaCl₂) can be extracted from natural brines and recovered as a by-product from chemical processes such as neutralization of hydrochloric acid with limestone/lime or from chlor-alkali operations. Besides CaCl₂), other materials with Ca like CaO and Ca(OH)₂ may also be used in this technology. To obtain CaCO₃ nanoparticles upon CO₂ conversion, centrifugation was used in this study. Similarly, in large scale industrial production processes, centrifugation may also be used to separate the nanoparticles form the amino acid solvent. Moreover, a spray tower contactor or a membrane may be used to separate the nanoparticles from the solvent.

Cytotoxicity studies indicated that CaCO₃ nanoparticles converted from CO₂ may present cytotoxicity toward BEAS-2B cells, depending on the nanoparticle concentration, exposure time, and if the nanoparticles were produced from RFG or SFG. At 10 mg/mL nanoparticle treatment, cell viability was higher than 60%. In general, higher concentration and longer exposure time led to higher toxicity (FIGS. 19A-20C). Previous studies have reported that CaCO₃ nanoparticles generally exhibit low toxicity in lung epithelial cells at concentrations up to 50 mg/mL. Interestingly, CeO₂ has also generally been considered biocompatible at lower concentrations. However, a recent study demonstrated significant cytotoxicity of SiO₂ nanoparticles at a concentration of 10 mg/mL after 6 hours of exposure, a finding comparable to what was observed with CaCO₃ nanoparticles in this study. These findings suggest that although CaCO₃ and similar nanoparticles generally have high biocompatibility, their toxicity is depending on various conditions, such as particle aggregation and sedimentation. Upon visual inspection, CaCO₃ nanoparticles were settled at the bottom of the wells at higher concentrations. This sedimentation may have resulted in an accumulation of nanoparticles at the cell surface, contributing to a decrease in cell viability. Additionally, partial dissolution of the nanoparticles could have released Ca ions and a release of CO₃⁻, which could alter the pH leading to damaging effects to the cells. A similar mechanism is described for the toxicity of zinc oxide (ZnO) nanoparticles, where the toxicity was related to the dissolution and release of zinc ions, leading to cell damage.

Although the exact mechanism underlying the reduced viability of BEAS-2B cells upon exposure to CaCO₃ nanoparticles has not been experimentally validated, previous studies suggested that the toxicity of nanoparticles and ultrafine particles is closely linked to their physicochemical properties, which can induce lung inflammation upon inhalation. Given their small size, CaCO₃ nanoparticles may behave similarly to dust, potentially causing irritation to the respiratory mucosa. When airborne, these nanoparticles can deposit in the lungs, potentially increasing oxidative stress through the production of reactive oxygen species. Studies in the future may elucidate the specific mechanisms contributed to the observed reduction in cell viability.

### Conclusion

In this study, Gly salt has been used to capture and mineralize CO₂ from flue gas into CaCO₃ nanoparticles which is widely applied in industrial as a filler material and biomedical fields for drug delivery. These findings indicated that the size and shape of CaCO₃ particles were significantly influenced by initial CO₂ loading levels, and Gly and carbamate amounts. By adjusting reaction parameters, the ability to tailor the nanoparticle production process to accommodate flue gases with varying CO₂ concentrations was demonstrated, highlighting the adaptability of the disclosed method. The application of the disclosed process using flue gases from power plants, despite their complex compositions, yielded nanoparticles with similar sizes and shapes as those from simulated flue gases, underscoring the robustness of our approach. Overall, this research contributes to the growing body of knowledge on carbon management technologies, offering a promising pathway for converting CO₂ into valuable nanomaterials.

### Example 5: Use of CaO in Combination with Amino Acid Salts to Convert CO₂ to CaCO₃ Under Mild pH and Low Temperature

### Experimental Section

Materials: L-glycine (Gly, ≥98 wt. %), sodium hydroxide (NaOH, ≥98 wt. %), calcium oxide (CaO, reagent grade), deuterium oxide (D₂O, ≥99.9 wt. %), and 1,4-dioxane (≥99.5 wt. %) were purchased from MilliporeSigma (Burlington, MA). CO₂ (99.99%) and nitrogen (N₂, ultra-high purity) were obtained from Airgas (Radnor, PA). All materials were used as received.

Methods: Amino acid salt solvents for CO₂ capture were prepared by mixing Gly, the simplest, inexpensive, and stable amino acid, with 0.025 mol NaOH at various molar ratios (0:1, 1:1, 2:1, and 3:1) in 25 ml of water in a 50 ml tube. The mixtures were placed in a water bath preheated to 60° C. and kept for 20 min. Although flue gas may contain many trace components (e.g. NO_{X}, SO₂, H₂S), its main constitutes, CO₂, N₂, H₂O and O₂, typically account for more than 99% of the total volume. In this study, H₂O and O₂ were excluded from consideration as they do not react with the solvent. Therefore, simulated flue gas containing 12% CO₂ (coal-fired power plant) was obtained by mixing CO₂ at a flow rate of 24 standard cubic centimeter per minute (sccm) with N₂ at 176 sccm and utilized as CO₂ source. A serological pipette was inserted into the tube to serve as the flue gas outlet. After 15 min of flue gas bubbling, 0.009 mol (0.517 g) CaO was added to the solvent, and the mixture was vortexed for 1 min. The tube was then centrifuged at 4,000 revolutions per minute for 30 min to collect the CaCO₃, which was subsequently washed and centrifuged three times. The amino acid salt solvent was reused without any additional treatment for subsequent cycles of CO₂ capture and mineralization.

The CO₂ loading of the solvent was determined by weighing the solvent during the bubbling process, following the method described in previous publications. The mass of CaCO₃ was measured using an analytical balance after 48 h of vacuum drying at 50° C. The pH value of the solvents was measured with a pH meter (Apera MP511).

The CO₂ loading was quantified relative to the NaOH dose (0.025 mol), expressed as CO₂ (mol) to OH⁻ (mol). The relative concentration of Gly was described by the peak area (1+2) of Gly to the peak area of reference. The yield of CaCO₃ was expressed as the molar ratio of CaCO₃ to CaO multiplied by 100%.

### Characterization

¹³C Nuclear Magnetic Resonance (¹³C NMR): An NMR instrument (Agilent 400-MR DD2, US) was utilized to obtain ¹³C NMR spectra, which were used to identify carbon species. For solvent analysis, a mixture of 500 μL sample, 200 μL D₂O, and 15 μL 1,4-dioxane was combined in a 1 mL centrifuge tube and then transferred to an NMR tube. D₂O was used as the lock and 1,4-dioxane as the internal reference. The ¹³C NMR tests were performed with 256 scans. To determine if chemicals like Gly might bind to the surfaces of the precipitates, 10 mg of the precipitates were mixed with 700 μL D₂O and 15 μL 1,4-dioxane and vortexed for 30 sec, and then the upper solution was transferred to an NMR tube for analysis. According to previous studies, the peaks in the NMR spectra were numerically labeled, and their corresponding assignments were detailed as follows. Peaks 1 and 2 correspond to the two carbons (CH₂, C═O) of Gly. Peaks 3, 4, and 5 represent the three carbons (CH₂, C═O, C═O) of carbamate, while peak 6 was attributed to HCO₃⁻/CO₃²⁻. The reference peak for 1,4-dioxane was observed at 67.19 ppm.

Electron Microscopy: The dried CaCO₃ samples were placed onto a stainless-steel disc and coated with a thin layer of gold using a Denton Desk V Sputter Coater for 240 sec. Scanning electron microscopy (SEM) images of CaCO₃ were captured using a SEM (JSM-7600F, Japan) at an acceleration voltage of 5 kV. For transmission electron microscopy (TEM) analysis, the CaCO₃ samples were suspended in ethanol and sonicated for 30 min before TEM (JEM-2100F, Japan) imaging on a copper grid.

X-ray Diffraction (XRD): Dried CaCO₃ samples were analyzed using a PANalytical X-ray diffractometer operated (X'Pert Pro, Netherlands) at 40 kV and 25 mA. Diffraction patterns were collected across a 20 range of 5° to 80° to determine the crystalline phases.

Statistical Analysis: Data with standard error bars were obtained in triplicate. Statistical analyses were conducted using JMP software. Statistical significance was determined at a p-value<0.05 through analysis of variance (ANOVA) followed by Tukey's Honest Significant Difference (HSD) test.

Control of Solvent pH and Mineralization of CO₂ into CaCO₃ Nanoparticles Using Gly/NaOH Molar Ratio of 3:1

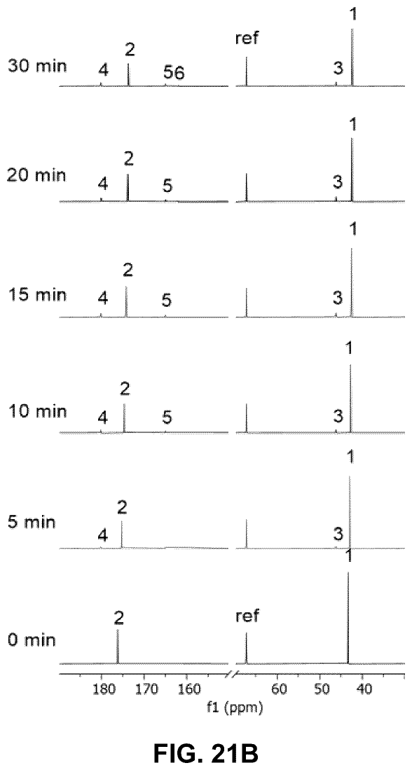

CO₂ capture: Amino acid salt solvent of a Gly/NaOH molar ratio of 3:1 in 25 ml of water was bubbled with flue gas for 30 min. During the bubbling process, the CO₂ loading increased steadily and reached a plateau of 0.369 mol/(mol OH⁻) after 15 min (FIG. 21A). The pH of the solvent decreased throughout the bubbling process, with a more rapid drop observed in the first 15 min (from 8.8 to 8.1) followed by a slower decline in the subsequent 15 min (from 8.1 to 8.0) (FIG. 21A).

The NMR spectra (FIG. 21B) showed that only peaks (1 and 2) representing Gly/Gly⁻ were present before flue gas bubbling. Peaks corresponding to carbamate began to form after 5 min of bubbling. By the 30-min mark, a small peak (6) representing HCO₃⁻/CO₃²⁻-appeared, though its intensity remained low. These NMR findings confirmed that the reaction pathway was consistent with the previous studies using a 1:1 molar ratio of Gly to NaOH/KOH and pure CO₂ as the carbon source. The primary reactions are as follows: (R1) The base NaOH deprotonates Gly to enable Gly ready to react with CO₂, (R2) the deprotonated Gly reacts with CO₂ to form carbamate, and (R3) carbamate is hydrolyzed to HCO₃⁻ and deprotonated Gly.

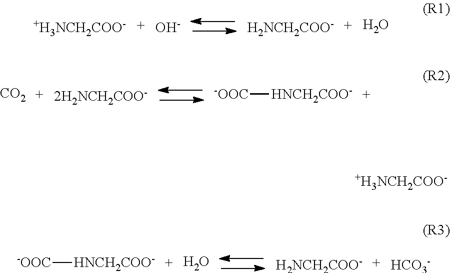

The NMR data also confirmed that the CO₂ capture step was rapid, whereas the carbamate hydrolysis step proceeded much more slowly.

CO₂ conversion using CaO: The highest CO₂ loading in the system was observed at 15 min, with a loading of 0.369 mol/(mol OH⁻) (FIG. 21A). If all captured CO₂ can be converted to CaCO₃, 0.009 mol of CaO would be required based on the following reaction (R4). Therefore, 0.009 mol (0.517 g) CaO was added to the solvent after 15 min of flue gas bubbling.

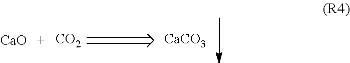

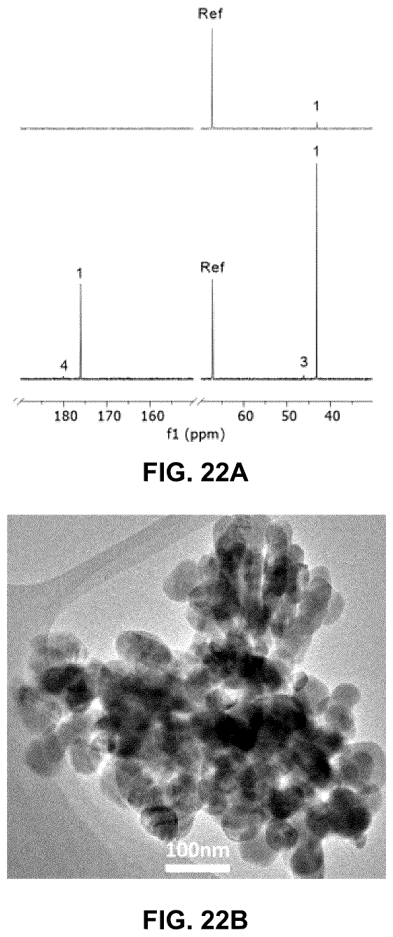

After the addition of CaO (FIG. 22A), the NMR spectra revealed that peak 6, representing HCO₃⁻/CO₃²⁻, disappeared. Peak 5, corresponding to one carbon (C═O) of carbamate, also disappeared, while the intensities of peaks 3 and 4, representing the other carbamate carbons (CH₂, C═O), significantly weakened. These results indicated that both HCO₃⁻/CO₃²⁻ and carbamate were consumed during the reaction. Based on the data and previous studies, HCO₃⁻/CO₃²⁻ reacted with CaO to form CaCO₃ and OH⁻, which further enabled the hydrolysis of carbamate. The primary reactions are as follows:

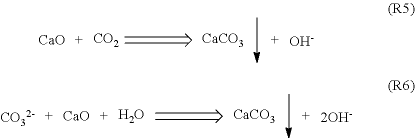

Trace amounts of Gly/Gly⁻ (peak 1) were detected in the solution samples from washing the precipitates, suggesting that Gly/Gly⁻ adhered to the surfaces of the CaCO₃ particles (FIG. 22A). TEM and SEM images (FIG. 22B) demonstrated that CaCO₃ particles formed were approximately 50 nm in size.

pH change during the whole process: The pH of the solvent is a critical parameter that significantly influences facility reliability and operational safety. In the pH 8-10 range, the passive film on stainless steel is generally more stable and less susceptible to breakdown compared to more acidic or highly alkaline environments. Here, when the solvent was placed in water bath (60° C.), the pH was 8.9 before bubbling and dropped to 8.3 after 15 min of flue gas bubbling; after the addition of 0.009 mol CaO, the pH returned to 8.9 (Table 3). When measured at room temperature (25° C.), the pH values were higher at all stages (Table 3): 9.8 before bubbling, 9.0 after 15 min of bubbling, and 9.8 after the addition of CaO. Overall, the pH of the solvent remained between 8.0 and 10.0, regardless of whether the solvent was capturing CO₂ or not. The pH change trend also supported the proposed reactions (R1-R6), indicating that OH⁻ was consumed during CO₂ capture, the captured CO₂ was mineralized, and OH⁻ was regenerated after the addition of CaO. Those results indicated that whether the solvent was in use at 60° C. or while stored at room temperature (i.e., 25° C.), the pH remained within a range that might cause minimal corrosion to the reactors.

TABLE 3
pH of solvent before bubbling, after 15 min bubbling, and after
CaO was added measured at 60° C. and 25° C.^{a}

pH value (average ± standard deviation)

| Temperature | Before | After 15 min | After adding |
|---|---|---|---|
| (° C.) | bubbling | bubbling | CaO |
| 60 | 8.9 ± 0.2 | 8.3 ± 0.3 | 8.9 ± 0.1 |
| 25 | 9.8 ± 0.0 | 9.0 ± 0.0 | 9.8 ± 0.2 |

^{a}Experimental conditions: NaOH (0.025 mol), Gly (0.075 mol), flue gas (12% CO₂), CaO (0.009 mol), and bubbling time (15 min).

Comparison of Solvent with Gly/NaOH Ratio from 0:1 to 3:1

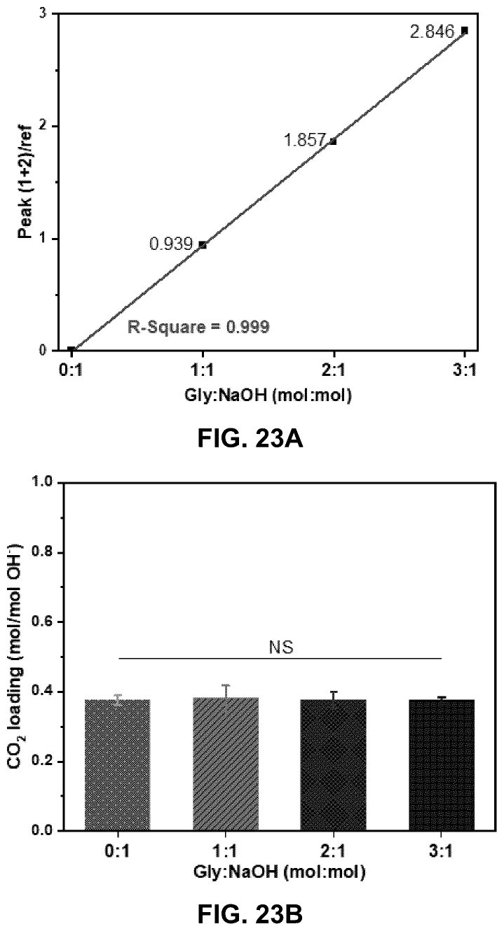

CO₂ capture: NMR spectra showed that no peaks, except for the reference peak, appeared when no Gly was used. When Gly was introduced at ratios from 1:1 to 3:1, peaks (1 and 2) representing Gly were observed. The peak intensities (1 and 2) were linearly related to the Gly dose (FIG. 23A). Peak 1 shifted from 45 ppm to 43 ppm, while peak 2 shifted from 182 ppm to 176 ppm, reflecting the changes in the ratio of Gly to Gly. NMR spectra demonstrated that, after bubbling the solvent for 15 min, only peak 6, representing HCO₃⁻/CO₃²⁻, was present when no Gly was used (Gly:NaOH=0:1). However, when Gly was used (Gly:NaOH=1:1, 2:1, 3:1), carbamate peaks were observed, while HCO₃⁻/CO₃²⁻ peaks decreased or even disappeared as more Gly was used, indicating that the reaction pathways were fundamentally different in the solvents with and without Gly. The reaction pathway involving Gly was discussed in R1, R2, and R3. The reaction pathway without amino acids is likely straightforward: CO₂ reacts with OH⁻ to form HCO₃⁻, which may further react with more OH⁻ to form CO₃²⁻.

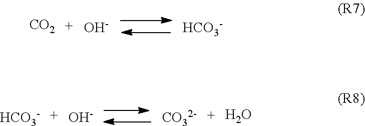

Although the dose of Gly influenced the ratio of Gly to Gly, the reaction pathway, and production of CO₃²⁻/HCO₃⁻, it did not influence the CO₂ loading (FIG. 23B).

After addition of CaO: As all solvents had the same CO₂ loading regardless of whether Gly was used or not (FIG. 23B), 0.009 mol of CaO was added to each solvent after 15 min of bubbling to mineralize the captured CO₂ and regenerate the solvent. Little HCO₃⁻/CO₃²⁻ remained after CaO was added to the solvent without Gly, and no peaks indicating HCO₃⁻/CO₃²⁻ were observed after CaO was added to the solvent containing Gly. Peaks (4 and 5) disappeared, while peak 3 remained but became weaker. The CO₂ mineralization and OH⁻ regeneration pathway involving Gly were discussed in reactions R1-R6. The reaction pathway without Gly was likely straightforward: HCO₃⁻/CO₃²⁻ reacted with CaO to form CaCO₃ and OH, as previously described in reactions R5 and R6.

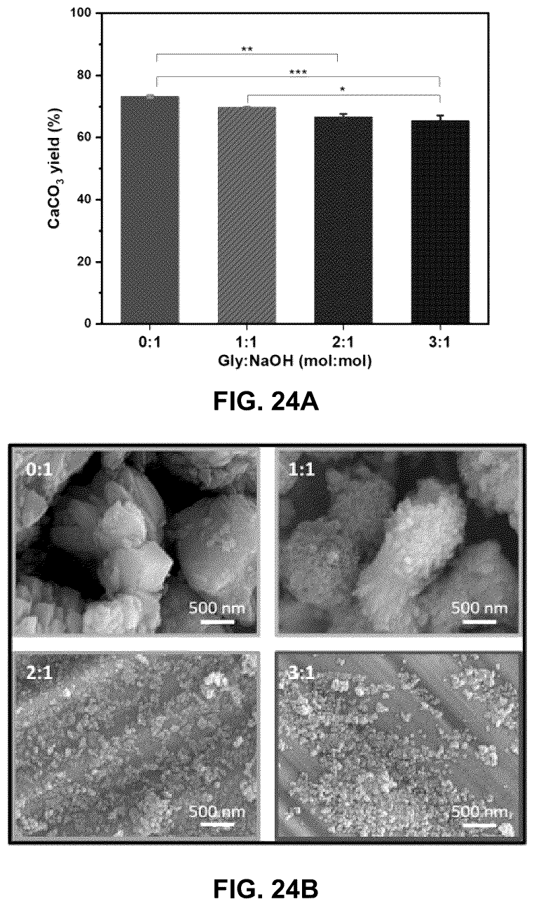

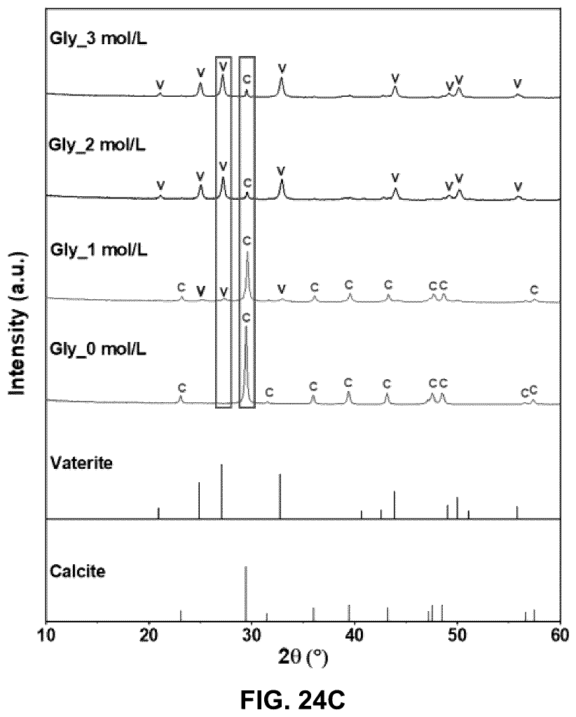

FIG. 24A showed that the CaCO₃ yield varied, with higher Gly levels resulting in relatively lower CaCO₃ yield. The size of the CaCO₃ particles differed significantly (FIG. 24B). Without Gly, the particles were relatively large with a wide size distribution (0.1-1 μm). In the presence of Gly, the particles became much smaller with a more uniform size distribution (~50 nm). However, when the Gly/NaOH ratio was 1:1, the particles aggregated, forming large porous structures. XRD spectra (FIG. 24C) revealed the phase changes. As the Gly content increased, the phase transitioned from calcite to vaterite, indicating that Gly played a crucial role in controlling the phase of CaCO₃ during synthesis. Without Gly, the CaCO₃ formed was calcite. With Gly, calcite was dominant at Gly/NaOH ratio of 1:1 while vaterite was dominant at Gly/NaOH ratios of 1:2 and 1:3 (FIG. 24C). The increased presence of vaterite observed at higher Gly/NaOH ratios can be attributed to the modulatory role of Gly in the CaCO₃ crystallization process. This finding is important, as different phases of CaCO₃ may have distinct industrial applications. For instance, while calcite is the most commonly used form of CaCO₃, vaterite holds great potential as sorbents for heavy metal ion removal.

pH change during the whole process: The pH values were measured at three timepoints: before flue gas bubbling, after 15 min of flue gas bubbling, and after the addition of CaO, all at a temperature of 60° C. In general, as the Gly concentration increased, the pH decreased at each respective timepoint. Without Gly, the pH was 12.1 before flue gas bubbling, dropped to 11.6 after 15 min of bubbling, and rose to 11.9 after the addition of CaO (Table 4). When the Gly dose was 0.025 mol, the pH was 10.8 before flue gas bubbling, dropped to 8.7 after 15 min of bubbling, and returned to 11.0 after the addition of CaO (Table 4). When the Gly dose was 0.050 mol, the pH was 9.2 before flue gas bubbling, dropped to 8.4 after 15 min of bubbling, and recovered to 9.2 after the addition of CaO (Table 4). When the Gly dose was 0.075 mol, the pH was 8.9 before bubbling and dropped to 8.3 after 15 min of flue gas bubbling, and after the addition of 0.009 mol CaO, the pH returned to 8.9 (Table 4).

These pH changes further confirmed the reaction pathway, demonstrating that OH⁻ was regenerated after the addition of CaO. In the presence of Gly, the pH was almost fully recovered, while in the absence of Gly, the pH was partially reversed (Table 4).

Industrial CO₂ capture systems must address the dual challenges of acidic and alkaline corrosion. While acidic environments readily attack steel surfaces, excessively high pH levels can lead to alkaline embrittlement, stress corrosion cracking, and damage to passivation films at elevated temperatures. Amino acid salts, typically prepared by combining amino acids (e.g., Gly, alanine, or sarcosine) with a strong base (e.g., NaOH or KOH) in a 1:1 molar ratio, are promising for CO₂ capture. Such amino acid salts normally result in a relatively high pH of approximately 12, as confirmed in this study, and such a high pH presents operational challenges, motivating efforts to identify and optimize solvent formulations. A mild alkaline environment (pH 8-10), obtained in this study, is ideal, as it facilitates the formation of stable oxide passivation films (e.g., Fe₃O₄ or Cr₂O₃), significantly reducing corrosion rates, and minimizing operational risks for workers.

TABLE 4
pH of solvent with different Gly doses at the timepoints
before flue gas bubbling, after 15 min flue gas bubbling,
and after the addition of CaO^{a}

pH value (average ± standard deviation)

| Gly:NaOH | Before | After 15 min | After adding |
|---|---|---|---|
| (mol:mol) | bubbling | bubbling | CaO |
| 0:1 | 12.1 ± 0.1 | 11.6 ± 0.2 | 11.9 ± 0.1 |
| 1:1 | 10.8 ± 0.1 | 8.7 ± 0.2 | 11.0 ± 0.2 |
| 2:1 | 9.2 ± 0.2 | 8.4 ± 0.1 | 9.2 ± 0.2 |
| 3:1 | 8.9 ± 0.2 | 8.3 ± 0.3 | 8.9 ± 0.1 |

^{a}Experimental conditions: NaOH (0.025 mol), Gly (0, 0.025, 0.050, 0.075 mol), flue gas (12% CO₂), bubbling time (15 min), and temperature (60° C.).

It has been well established that crystal nucleation and growth (influenced by pH, temperature, additives, ion concentration, etc.) determine the morphology of CaCO₃. When Ca²⁺ is introduced into the solution, it can form a coordination complex with Gly if present, and Gly can also be adsorbed onto the surface of CaCO₃. Both interactions disrupt the nucleation and growth of CaCO₃. Furthermore, these results align with previous studies, which demonstrated that lower pH promotes the formation of vaterite and inhibits its transformation into calcite.

In summary, Gly could be a potential candidate for controlling the size and phase of CaCO₃ and mitigating solvent corrosion. When the ratio of Gly/NaOH exceeded 2, the changes in solvent pH, particle size and phase of CaCO₃, and CaO conversion difference became negligible.

Recycling and Reusing Gly to Mineralize CO₂ into CaCO₃ Nanoparticles

Recycling and reusing Gly was the core of this method, as it could significantly reduce the costs for raw materials and operation, making the method potentially suitable for industrial applications. To evaluate the stability of the solvent, cyclic usage was conducted without any treatment, using a solvent with a Gly/NaOH ratio of 3:1.

Solvent change during cycling: Data showed that Gly (peaks 1 and 2) remained the dominant component in the solvent after each cycle. The remaining carbamate (peaks 3 and 4) could only be observed after magnifying the NMR spectra. The consistent intensity of peaks 3 and 4 after the cycles indicated that the solvent remained stable following the cycles.

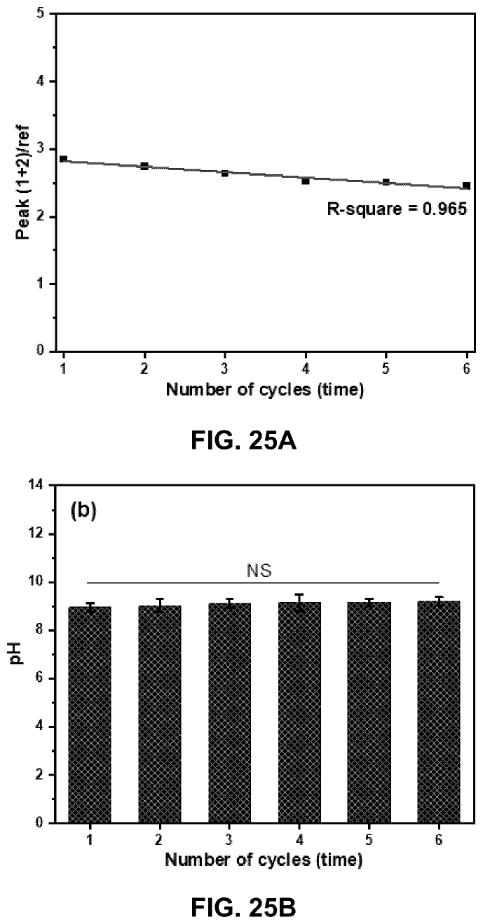

According to FIG. 23A, the ratio of peak area (1+2)/ref was linearly correlated with the Gly concentration. As depicted in FIG. 25A, this ratio gradually decreased with each cycle, indicating a nearly constant although small Gly loss during the cyclic process. This loss was due to the adherence of Gly to the surfaces of CaCO₃ nanoparticles as evidenced by the NMR data (FIG. 22A). It is worth noting that the Gly physically adhered to the particle surfaces can be reclaimed through simple methods, such as washing the particles with water. Alternatively, intentionally retaining Gly on the surface of the CaCO₃ particles can be considered for two main reasons: (1) Gly is environmentally friendly and biodegradable, and (2) due to their small size, nanoparticles are prone to aggregation, and the adsorption of glycine onto the particle surfaces can effectively inhibit this aggregation, thereby enhancing particle stability. FIG. 25B showed that there was no obvious change in the solution pH over the course of the cycles.

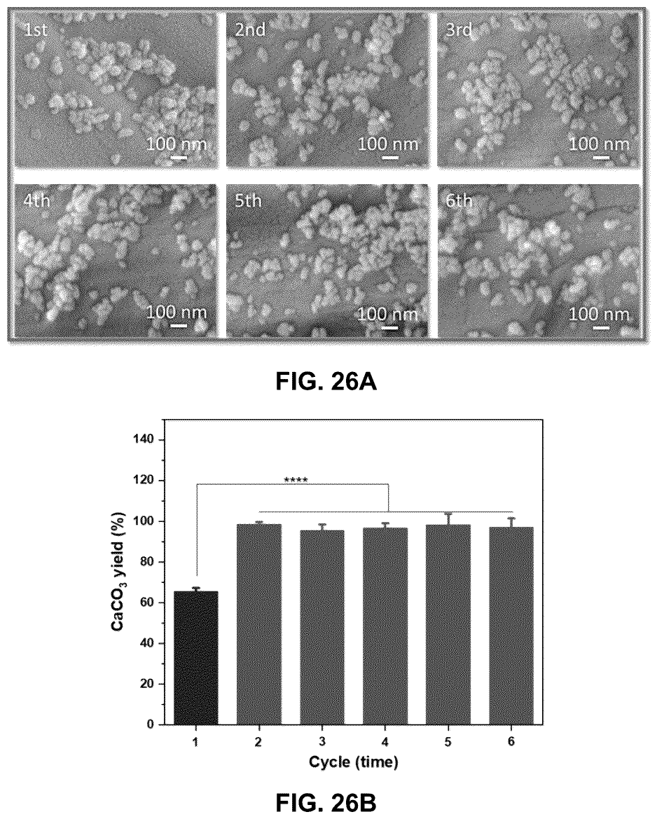

Product observation during cycling: FIG. 26A showed that the size of CaCO₃ particles remained unchanged across cycles, with a uniform distribution around 50 nm. The NMR spectra of the solution samples from washing the CaCO₃ precipitates presented a consistent presence of Gly indicating the attachment of Gly to the particles. This finding aligned with the small solvent changes shown in FIG. 25A. FIG. 26B revealed that the conversion of CaO in the first cycle was approximately 65%, which was significantly lower than that in subsequent cycles. After the first cycle, the system stabilized, allowing nearly complete conversion of CaO to CaCO₃ nanoparticles.

The cycling experiments demonstrated the reliability of the solvent for recycling and reuse in capturing CO₂ and producing CaCO₃ nanoparticles while simultaneously regulating the solvent's pH value. Although some loss of Gly is inevitable due to its tendency to adhere to the surfaces of CaCO₃ nanoparticles, this environmentally friendly and biodegradable adsorption helps prevent the nanoparticles which tend to aggregate easily because of their small size from clustering, thereby improving particle stability. The results clearly indicated that after the first cycle, the system became stabilized and the CaO conversion (FIG. 26B) reached consistently high levels (~100%). The conversion in the first cycle was not 100% because not all of the CO₂ in the carbamate was converted to CaCO₃, due to the presence of chemical equilibria in the system. In other words, the system had not yet reached equilibrium during the first cycle.

### Practical Applications and Future Research Prospects

The disclosed method for capturing and converting CO₂ into CaCO₃ nanoparticles has been demonstrated to be feasible. Moreover, a phase transition of CaCO₃ from calcite to vaterite was observed. This finding is significant, as different polymorphs of CaCO₃ exhibit distinct properties and serve various industrial applications. For example, calcite, the most thermodynamically stable form, is commonly used as a functional filler in industries such as plastics, rubber, paints, and paper. In contrast, vaterite shows great potential in advanced applications including heavy metal ion adsorption, drug delivery, bone tissue engineering, and cosmetic formulations.

Although trace components such as SO₂ and NOₓ are typically present in low concentrations in real flue gas, they may still interact with the active components in the present system, potentially affecting its long-term stability and overall efficiency. Therefore, future studies should incorporate more industrial flue gas—such as those from power plants, cement production facilities, and steel plants—to better evaluate the performance and durability of this method under practical operating conditions.

While this approach shows promise as an alternative to conventional amine-based CO₂ capture and as a potential contributor to the nanoparticle production industry, several specific factors must be addressed for successful industrial application. These include selecting appropriate materials for corrosion-resistant equipment compatible with the disclosed solvent, optimizing the process to prevent CaCO₃ product buildup or blockages in the system, and conducting comprehensive life cycle assessment (LCA) and techno-economic analysis (TEA) to ensure viability at scale.

### Conclusion

This study presented a sustainable and efficient strategy for capturing and mineralizing CO₂ from flue gas into CaCO₃ nanoparticles using CaO in combination with amino acid salt where Gly was applied to optimize the solvent pH and control the morphology of CaCO₃. This environmentally friendly and economically viable approach not only promoted the transition to sustainable industrial practices but also contributed to global carbon management efforts. The process effectively addressed key challenges such as solvent stability, corrosion risks, and product value, maintaining a mild alkaline pH range (8-10) to minimize reactor corrosion and enhance mineralization efficiency. The resulting product (i.e., CaCO₃ nanoparticles), with a uniform particle size of approximately 50 nm and tunable phases, holds significant industrial potential, further supported by the solvent's recyclability without additional treatment.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Ababneh, H., et al, 2022. Carbon capture from post-combustion flue gas using a state-of-the-art, anti-sublimation, solid-vapor separation unit. Processes, 10, 2406.
2. Adu, E., et al, 2020. Parametric process design and economic analysis of post-combustion CO₂ capture and compression for coal- and natural gas-fired power plants. Energies, 13, 2519.
3. Alavi, M. A.; et al. Syntheses of BaCO₃ nanostructures by ultrasonic method. Ultrasonics sonochemistry 2008, 15 (5), 833-838.
4. Alper, E et al., CO₂ utilization: Developments in conversion processes, Petroleum, 3 (2017) 109-126.
5. Armstead, A. L., et al, 2015. Acute inflammatory responses of nanoparticles in an intra-tracheal instillation rat model, PloS one 10, e0118778/6.
6. Armstead, A L et al., Nanotoxicity: Emerging concerns regarding nanomaterial safety and occupational hard metal (WC-Co) nanoparticle exposure, Int. J. Nanomed. (2016) 6421-6433.
7. Armstead, A L et al, Exploring the potential role of tungsten carbide cobalt (WC-Co) nanoparticle internalization in observed toxicity toward lung epithelial cells in vitro, Toxicol. Appl. Pharmacol. 278 (2014) 1-8.
8. Armstead, A. L.; et al. Toxicity and oxidative stress responses induced by nano- and micro-CoCrMo particles. Journal of materials chemistry B 2017, 5 (28), 5648-5657.
9. Bachu, S. Identification of oil reservoirs suitable for CO₂-EOR and CO₂ storage (CCUS) using reserves databases, with application to Alberta, Canada, Int. J. Greenhouse Gas Control 2016, 44, 152-165.
10. Bai, B.-k.; et al. Infectious disease surveillance in China. Journal of Infection 2015, 71 (6), 698-700.
11. Baig, N.; et al. Nanomaterials: A review of synthesis methods, properties, recent progress, and challenges. Materials Advances 2021, 2 (6), 1821-1871.
12. Baker, R W et al, CO₂ capture from cement plants and steel mills using membranes, Ind. Eng. Chem. Res. 57 (2018) 15963-15970.
13. Bao, Z et al., Innovative cycling reaction mechanisms of CO₂ absorption in amino acid salt solvents, Chem. Eng. J. Adv. (2022) 100250.
14. Barker, R., 1973. The reversibility of the reaction CaCO₃⇄CaO+CO₂, J. Appl. Chem. Biotechnol. 23, 733-742.
15. Bandilla, K. W. Carbon capture and storage. In Future energy, Elsevier, 2020; pp 669-692.
16. Betts, J. W.; et al. Novel antibacterials: alternatives to traditional antibiotics. In Advances in microbial physiology, Vol. 73; Elsevier, 2018; pp 123-169.
17. Black, M. S.; et al. Not yet on track to net zero: The urgent need for greater ambition and policy action to achieve Paris temperature goals; International Monetary Fund, 2021.
18. Brandi, C.; et al. The role of carbon dioxide therapy in the treatment of chronic wounds. in vivo 2010, 24 (2), 223-226.
19. Buckley, J. J.; et al. Silver carbonate nanoparticles stabilised over alumina nanoneedles exhibiting potent antibacterial properties. Chemical communications 2008, (34), 4013-4015.
20. Bui, M.; et al. Carbon capture and storage (CCS): the way forward. Energy & Environmental Science 2018, 11 (5), 1062-1176.
21. Burkart, M. D.; et al. Opportunities and challenges for catalysis in carbon dioxide utilization. ACS Catalysis 2019, 9 (9), 7937-7956.
22. Cao, M., et al, 2019. Effect of macro-, micro- and nano-calcium carbonate on properties of cementitious composites—A review. Materials, 12, 781.
23. Centers for Disease Control and Prevention, Antibiotic resistance threats in the United States. US Department of Health and Human Services: Washington, DC, USA 2019, 1, 67-100.
24. Chao, C., et al, 2021. Post-combustion carbon capture. Renewable Sustainable Energy Rev. 138, 110490.
25. Cortes, V et al., Economics of Direct Air Capture of Carbon Dioxide, Master's Project, Duke University, Durham, NC, 2020.
26. Courtney, C. M.; et al. Photoexcited quantum dots for killing multidrug-resistant bacteria. Nature materials 2016, 15 (5), 529-534.
27. Cui, X et al., A cluster-nanozyme-coenzyme system mimicking natural photosynthesis for CO2 reduction under intermittent light irradiation, Nat. Commun. 15 (2024) 9048.
28. Cui, X et al., Modulating carbon dioxide activation on carbon nanotube immobilized salophen complexes by varying metal centers for efficient electrocatalytic reduction, J. Colloid Interface Sci. 608 (2022) 1827-1836.
29. Dalisson, B.; et al. Bioinorganics and wound healing. Advanced Healthcare Materials 2019, 8.
30. Dang, H., et al, 2017. Facile batch synthesis of porous vaterite microspheres for high efficient and fast removal of toxic heavy metal ions, J. Environ. Chem. Eng. 5, 4505-4515.
31. Dawadi, S. et al. Current research on silver nanoparticles: synthesis, characterization, and applications. Journal of nanomaterials 2021, 2021, 1-23.
32. Dedman, A., et al, 1962. Calcium cyanamide synthesis. Part 4.—the reaction CaO+ CO₂═CaCO₃, Trans. Faraday Soc. 58, 2027-2035.
33. Deng, Q et al., CCS and CCUS technologies: Giving the oil and gas industry a green future, Front. Energy Res. 10 (2022) 919330.
34. Ding X, T. Challenges and innovations in treating chronic and acute wound infections: from basic science to clinical practice [J/OL]. Burns Trauma, 2022, 10: tkac014 [2022 Apr. 7].
35. Duan, Y., et al, 2014. ab initio thermodynamic study of the CO₂ capture properties of M₂CO₃ (M=Na, K)- and CaCO₃-promoted MgO sorbents towards forming double salts, Aerosol Air Qual. Res., 14, 470-479.
36. EPICA community members. Eight glacial cycles from an Antarctic ice core, Nature 429 (2004) 623-628.
37. Fu, L et al., Research progress on CO₂ capture and utilization technology, J. CO₂ Util. 66 (2022) 102260.
38. Fu, Q., et al, 2022. Effect of nano calcium carbonate on hydration characteristics and microstructure of cement-based materials: a review, J. Build. Eng., 50, 104220.
39. Gao, W et al., Industrial carbon dioxide capture and utilization: State of the art and future challenges, Chem. Soc. Rev. 49 (2020) 8584-8686.
40. Garrabrant, K. A., et al, 2019. Energy-efficient CO₂ capture from flue gas by absorption with amino acids and crystallization with a bis-iminoguanidine, Ind. Eng. Chem. Res. 58, 10510-10515.
41. Gaurina-Me\vec{d}imurec, N.; et al. Carbon capture and storage (CCS): Geological sequestration of CO₂. CO₂ Sequestration 2019, 1-21.
42. Gayathri, R et al., Carbon dioxide capture and bioenergy production by utilizing the biological system, In: M. Jawaid, A. Khan (eds), Sustainable Utilization of Carbon Dioxide, Springer, Singapore, 2023, pp. 159-194.
43. Geerlings, H., et al, 2013. CO₂ mineralization-bridge between storage and utilization of CO₂, Annu. Rev. Chem. Biomol. Eng. 4, 103-117.
44. Ghazi, S.; et al. Ag₂CO₃ phases in nanocomposites with palygorskite and their effects on photocatalytic and antibacterial activities. Applied Clay Science 2024, 252, 107342.
45. Gherasim, O.; et al. M. An updated review on silver nanoparticles in biomedicine. Nanomaterials 2020, 10 (11), 2318.
46. Gibbins, J et al., Carbon capture and storage, Energy policy, 36 (2008) 4317-4322.
47. Grand View Research, 2023. Nano calcium carbonate market size, share & trends analysis report by application (plastics, rubber, building & construction), by region (asia pacific, north America, europe), and segment forecasts, 2023-2030. www.grandviewresearch.com/industry-analysis/nano-calcium-carbonate-market (accessed 4 Apr. 2025).
48. Guo, S.; et al. Controllable synthesis porous Ag₂CO₃ nanorods for efficient photocatalysis. Nanoscale Research Letters 2015, 10, 1-8.
49. Hembram, K. C.; et al. Therapeutic prospective of plant-induced silver nanoparticles: application as antimicrobial and anticancer agent. Artificial cells, nanomedicine, and biotechnology 2018, 46 (sup3), 38-51.
50. Herzog, H. Direct air capture, In: M. Bui, N. M. Dowell (Eds.), Greenhouse Gas Removal Technologies, The Royal Society of Chemistry, 2022; pp. 115-137.
51. Hou, R et al., Current status and advances in membrane technology for carbon capture, Sep. Purif. Technol. 300 (2022) 121863.
52. Huang, Y., et al, 2022. Protonated amines mediated CO₂ mineralization of coal fly ash and polymorph selection of CaCO₃, Chem. Eng. J. 450, 138121.
53. Ingvarsdóttir, A. Comparison of Direct Air Capture Technology to Point Source CO₂ Capture in Iceland, Master's Project, KTH Royal Institute of Technology, Stockholm, Sweden, 2020.
54. Islam, R.; et al. Biomedical applications of Chinese herb-synthesized silver nanoparticles by phytonanotechnology. Nanomaterials 2021, 11 (10), 2757.
55. Jiang, B., et al, 2013. Development of amino acid and amino acid-complex based solid sorbents for CO₂ capture, Appl. Energy. 109, 112-118.
56. Jiutian, Z.; et al. Several key issues for CCUS development in China targeting carbon neutrality. Carbon Neutrality 2022, 1 (1), 17.
57. Kasturi, A., et al, 2025. Sub-ambient performance of potassium sarcosinate for direct air capture applications: CO₂ flux and viscosity measurements. Sep. Purif. Technol. 357, 130026.
58. Kasturi, A., et al, 2021. Carbon dioxide capture with aqueous amino acids: mechanistic study of amino acid regeneration by guanidine crystallization and process intensification. Sep. Purif. Technol. 271, 118839.
59. Ketabchi, M R et al., Latest advances and challenges in carbon capture using bio-based sorbents: A state-of-the-art review, Carbon Capture Sci. Technol. 6 (2023) 100087.
60. Khabbaz, R. F.; et al. Challenges of infectious diseases in the USA. The Lancet 2014, 384 (9937), 53-63.
61. Khalilpour, R et al., Membrane-based carbon capture from flue gas: A Review, J. Cleaner Prod. 103 (2015) 286-300.
62. Khanjani, M., et al, 2021. Tuning polymorphs and morphology of microbially induced calcium carbonate: Controlling factors and underlying mechanisms. ACS omega 6, 11988-12003.
63. Khraisheh, M et al., Solid sorbents as a retrofit technology for CO₂ removal from natural gas under high pressure and temperature conditions, Sci. Rep. 10 (2020) 269.
64. Kierzkowska, A. M., et al, 2013. CaO-based CO₂ sorbents: from fundamentals to the development of new, highly effective materials. ChemSusChem 6, 1130-1148.
65. Kim, S et al., Gas-liquid membrane contactors for carbon dioxide separation: A review, Chem. Eng. J. 411 (2021) 128468.
66. Laska, C., et al, 2020. Economics of direct air capture of carbon dioxid, Duke University. hdl.handle.net/10161/20450
67. Lee, N.-Y.; et al. Nanoparticles in the treatment of infections caused by multidrug-resistant organisms. Frontiers in pharmacology 2019, 10, 452171.
68. Li, B.; et al. Advances in CO₂ capture technology: A patent review. Applied Energy 2013, 102 (C), 1439-1447.
69. Li, B., et al, 2011. Innovative nano-layered solid sorbents for CO₂ capture. Chem Commun. 47, 1719-1721.
70. Li, B., et al, Layered solid sorbents for carbon dioxide capture, U.S. Pat. No. 8,658,561B2.
71. Li, B., et al. Amino acids react with carbon dioxide (CO₂) and form nanofibers and nanoflowers. U.S. Pat. No. 10,583,388B2.
72. Li, N. et al., A work group report on ultrafine particles (American Academy of Allergy, Asthma & Immunology): Why ambient ultrafine and engineered nanoparticles should receive special attention for possible adverse health outcomes in human subjects, J. Allergy Clin. Immunol. 138 (2016) 386-396.
73. Li Q, et al. (2025). Amino acid-driven one-step process to transform CO₂ into barium carbonate nanoparticles. Submitted. ACS Sustain Chem Eng (IF=7.1) DOI: 10.1021/acssuschemeng.4c10207.
74. Li Q, et al. (2025). Capturing and converting CO₂ using amino acids as various commercially valuable nano-carbonates. Sep Purif Techn (IF=7.28) 361:131207.
75. Li, Q et al., Unraveling the role of glycine in K₂CO₃ solvent for CO₂ removal, Ind. Eng. Chem. Res. 61 (2022) 12545-12554.
76. Li, S et al., Recent advances and future perspectives of low-concentration CO₂ enrichment and emerging applications. Energy Fuels 39 (2025) 20056-20083.
77. Li, W.-P.; et al. CO₂ delivery to accelerate incisional wound healing following single irradiation of near-infrared lamp on the coordinated colloids. ACS nano 2017, 11 (6), 5826-5835.
78. Liendo, F., et al, 2022. Factors controlling and influencing polymorphism, morphology and size of calcium carbonate synthesized through the carbonation route: a review. Powder Technol. 398, 117050.
79. Liendo, F., et al, 2022. Nucleation and growth kinetics of CaCO₃ crystals in the presence of foreign monovalent ions. J. Cryst. Growth 578, 126406.
80. Lin, P., et al, 2020. Preparation of vaterite calcium carbonate granules from discarded oyster shells as an adsorbent for heavy metal ions removal. Chemosphere 254, 126903.
81. Liu, M., et al, 2021. Integrated CO₂ capture and removal via carbon mineralization with inherent regeneration of aqueous solvents. Energy Fuels 35, 8051-8068.
82. Liu, M.; et al. Single-step, low temperature and integrated CO₂ capture and conversion using sodium glycinate to produce calcium carbonate. Fuel 2020, 275, 117887.
83. Liu, W., et al, 2012. Synthesis of CaO-based sorbents for CO₂ capture by a spray-drying technique. Environ. Sci. Technol. 46, 11267-11272.
84. Lo, J. F.; et al. Microfluidic wound bandage: localized oxygen modulation of collagen maturation. Wound Repair and Regeneration 2013, 21 (2), 226-234.
85. Lončarević, D.; et al. Antimicrobial and Photocatalytic Abilities of Ag₂CO₃ Nano-Rods. ChemistrySelect 2017, 2 (10), 2931-2938.
86. Luis, P. Use of monoethanolamine (MEA) for CO₂ capture in a global scenario: Consequences and alternatives, Desalination 380 (2016) 93-99.
87. Lyu, Y. et al., Nano-calcium carbonate affect the respiratory and function through inducing oxidative stress: A cross-sectional study among occupational exposure of workers and a further research for underlying mechanisms, J. Occup. Environ. Med. 65 (2023) 184-191.
88. Ma, L., et al, 2021. Controllable crystallization of pure vaterite using CO₂-storage material and different Ca²⁺ sources, J. CO₂ Util., 48, 101520.
89. Madhav, D.; et al. Amino acid promoted single-step carbon dioxide capture and mineralization integrated with polymer-mediated crystallization of carbonates. Journal of Cleaner Production 2023, 137845.
90. Madl, A K et al., Nanoparticles, lung injury, and the role of oxidant stress, Annu. Rev. Physiol. 76 (2014) 447-465.
91. Maeda, M., et al, 1990. Complex formation of calcium (II) with amino acids under physiological conditions. J. Chem. Soc., Dalton Trans. 2337-2339.
92. Majeed, H et al., Characterization of aerosol emissions from CO₂ capture plants treating various power plant and industrial flue gases, Int. J. Greenhouse Gas Control 74 (2018) 282-295.
93. Malik, S.; et al. Nanotechnology: A revolution in modern industry. Molecules 2023, 28 (2), 661.
94. Mani, Z. A.; et al. Adapting disaster preparedness strategies to changing climate patterns in Saudi Arabia: A rapid review. Sustainability 2023, 15 (19), 14279.
95. Meckling, J et al., A policy roadmap for negative emissions using direct air capture, Nat. Commun. 12 (2021) 2051.
96. Mergenbayeva, S.; et al. Photocatalytic degradation of 4-tert-butylphenol using solar light responsive Ag₂CO₃. Catalysts 2022, 12 (12), 1523.
97. Messaoudi, O.; et al. Characterization of Silver Carbonate Nanoparticles Biosynthesized Using Marine Actinobacteria and Exploring of Their Antimicrobial and Antibiofilm Activity. Marine Drugs 2023, 21 (10), 536.
98. Natan, M.; et al. From nano to micro: using nanotechnology to combat microorganisms and their multidrug resistance. FEMS microbiology reviews 2017, 41 (3), 302-322.
99. Neerup, R., et al, 2023. Solvent degradation and emissions from a CO₂ capture pilot at a waste-to-energy plant. J. Environ. Chem. Eng., 11, 111411.
100. Niu, Y., et al, 2022. Calcium carbonate: controlled synthesis, surface functionalization, and nanostructured materials. Chem. Soc. Rev. 51, 7883-7943.
101. Nosrati, H.; et al. Nanocomposite scaffolds for accelerating chronic wound healing by enhancing angiogenesis. Journal of Nanobiotechnology 2021, 19, 1-21.
102. Ochedi, F O et al., Carbon dioxide capture using liquid absorption methods: A review. Environ. Chem. Lett. 19 (2021) 77-109.
103. Oda, T.; et al. Effects of the duration of transcutaneous CO₂ application on the facilitatory effect in rat fracture repair. Journal of Orthopaedic Science 2020, 25 (5), 886-891.
104. Ordomsky, V. V.; et al. Challenges and role of catalysis in CO₂ conversion to chemicals and fuels. Nanotechnology in Catalysis: Applications in the Chemical Industry, Energy Development, and Environment Protection 2017, 803-850.
105. Pan, N.; et al. A comparison study of the performance of Ag₂CO₃ photocatalysts synthesized respectively in a surfactant-free microemulsion, water, and a water/propan-2-ol mixture. Journal of Materiomics 2022, 8 (4), 781-789.
106. Pan, S.-Y.; et al. CO₂ mineralization and utilization by alkaline solid wastes for potential carbon reduction. Nature Sustainability 2020, 3 (5), 399-405.
107. Parangusan, H., et al, 2021. A review of passivity breakdown on metal surfaces: influence of chloride- and sulfide-ion concentrations, temperature, and pH. Emergent Mater. 4, 1187-1203.
108. Parani, M.; et al. Engineered nanomaterials for infection control and healing acute and chronic wounds. ACS Applied Materials & Interfaces 2016, 8 (16), 10049-10069.
109. Portugal, J. et al., Toxicity of airborne nanoparticles: Facts and challenges, Environ. Int. (2024) 108889.
110. Rafique, M.; et al. History and fundamentals of nanoscience and nanotechnology. In Nanotechnology and photocatalysis for environmental applications, Elsevier, 2020; pp 1-25.
111. Rickards, L.; et al. Implications of climate change for. part or in whole) and any information or material contained in it. Publisher: 2021.
112. Rivers, R. J.; et al. The tissue response to hypoxia: How therapeutic carbon dioxide moves the response toward homeostasis and away from instability. International journal of molecular sciences 2023, 24 (6), 5181.
113. Salleh, A.; et al. The potential of silver nanoparticles for antiviral and antibacterial applications: A mechanism of action. Nanomaterials 2020, 10 (8), 1566.
114. Santos, F. D.; et al. The climate change challenge: A review of the barriers and solutions to deliver a Paris solution. Climate 2022, 10 (5), 75.
115. Saravanan, A.; et al. A comprehensive review on different approaches for CO₂ utilization and conversion pathways. Chemical Engineering Science 2021, 236, 116515.
116. Scholes, C., et al, 2016. Membrane-cryogenic post-combustion carbon capture of flue gases from NGCC. Technol., 4, 14.
117. Schraufnagel, D E. The health effects of ultrafine particles, Exp. Mol. Med. 52 (2020) 311-317.
118. Shivkumara, C., et al, 2006. Synthesis of vaterite CaCO₃ by direct precipitation using glycine and L-alanine as directing agents. Mater. Res. Bull. 41, 1455-1460.
119. Singh, G et al., Emerging trends in porous materials for CO₂ capture and conversion, Chem. Soc. Rev. 49 (2020) 4360-4404.
120. Sivanesan, D.; et al. Enhanced CO₂ absorption and desorption in a tertiary amine medium with a carbonic anhydrase mimic. Journal of industrial and engineering chemistry 2017, 52, 287-294.
121. Song, K., et al, 2022. Phase and morphology of calcium carbonate precipitated by rapid mixing in the absence of additives. RSC Adv. 12, 19340-19349.
122. Soosaiprakasam, I. R., et al, 2008. Corrosion and polarization behavior of carbon steel in MEA-based CO₂ capture process. Int. J. Greenhouse Gas Control 2, 553-562.
123. Štajner, L., et al, 2018. The effect of different amino acids on spontaneous precipitation of calcium carbonate polymorphs. J. Cryst. Growth 486, 71-81.
124. Stambouli, M., et al, 2023. Effect of calcium carbonate particle size and content on the thermal properties of PVC foamed layer used for coated textiles. Turk. J. Chem. 47, 40-46.
125. Sullivan, I., et al, 2021. Coupling electrochemical CO₂ conversion with CO₂ capture. Nat. Catal. 4, 952-958.
126. Sun, S., et al, 2006. Interfacial structures and mechanical properties of PVC composites reinforced by CaCO₃ with different particle sizes and surface treatments. Polym. Int. 55, 158-164.
127. Tabrizi, E., et al, 2025. From innovation to application: safety concerns in nanomaterial implant coatings. Nanomedicine 20 (12), 1373-1376.
128. Tang, D., et al, 2015. Effect of pH value on corrosion of carbon steel under an applied alternating current. Mater. Corros. 66, 1467-1479.
129. Tan, A. W.; et al. Enhanced in vitro angiogenic behaviour of human umbilical vein endothelial cells on thermally oxidized TiO₂ nanofibrous surfaces. Scientific reports 2016, 6 (1), 21828.
130. Tang, S.; et al. Antibacterial activity of silver nanoparticles: structural effects. Advanced healthcare materials 2018, 7 (13), 1701503.
131. Thunberg, J C et al., Process for recovering glycine from sodium chloride solutions. U.S. Pat. No. 3,985,801A, 1976.
132. Tobler, D. J., et al, 2016. Effect of pH on amorphous calcium carbonate structure and transformation. Cryst. Growth Des. 16, 4500-4508.
133. Trushina, D. B., et al, 2014. CaCO₃ vaterite microparticles for biomedical and personal care applications. Mater. Sci. Eng., C 45, 644-658.
134. U.S. Energy Information Administration, Monthly Energy Review, Environment. www.eia.gov/tools/faqs/faq.php?id=77&t=11, 2023 (accessed 13 Jun. 2025).
135. U.S. Environmental Protection Agency, Inventory of U.S. greenhouse gas emissions and sinks: 1990-2021. www.epa.gov/ghgemissions/inventory-us-greenhouse-gas-emissions-and-sinks-1990-2021, 2023 (accessed 13 Jun. 2025).
136. Unveren, E E et al., Solid amine sorbents for CO₂ capture by chemical adsorption: A review, Petroleum 3 (2017) 37-50.
137. Vakili, M., et al, 2024. Analysis, assessment, and mitigation of stress corrosion cracking in austenitic stainless steels in the oil and gas sector: A review. Surfaces 7, 589.
138. Valverde, J., et al, 2012. Enhancement of fast CO₂ capture by a nano-SiO₂/CaO composite at Ca-looping conditions. Environ. Sci. Technol. 46, 6401-6408.
139. Wang, R. et al. TEA of a unique two-pathways process for post-combustion CO₂ capture. J. Energy Power Technol. 4 (2022) 033.
140. Wang, R. et al. Development of an innovative process for post-combustion CO₂ capture to produce high-value NaHCO₃ nanoparticles, Int. J. Greenhouse Gas Control 120 (2022) 103761.
141. Wang, X et al., Phase change amino acid salt separates into CO₂-rich and CO₂-lean phases upon interacting with CO₂, Appl. Energy 161 (2016) 41-47.
142. Wang, X et al., Unique biological amino acids turn CO₂ emission into novel nanomaterials with three switchable product pathways, Environ. Technol. Innovation 32 (2023) 103279.
143. Wang, X., et al, 2013. Amino acid-functionalized Ionic liquid solid sorbents for post-combustion carbon capture. ACS Appl. Mater. Interfaces 5, 8670-8677.
144. Wang, X., et al, 2013. Immobilization of amino acid ionic liquids into nanoporous microspheres as robust sorbents for CO₂ capture. J. Mater. Chem. A 1, 2978-2982.
145. Wang, Y.; et al. Antibiotic-free antibacterial strategies enabled by nanomaterials: progress and perspectives. Advanced Materials 2020, 32 (18), 1904106.
146. White, M T et al., Review of supercritical CO₂ technologies and systems for power generation, Appl. Therm. Eng. 185 (2021) 116447.
147. Wickramasinghe, S.; et al. Carbon dioxide conversion to nanomaterials: Methods, applications, and challenges. Energy & Fuels, 2021, 35 (15), 11820-11834.
148. Wu, H.-C.; et al. Microstructure of BaCO₃ and BaTiO₃ coatings produced on titanium by plasma electrolytic oxidation. Applied Surface Science 2020, 506, 144858.
149. Xia, T. et al., Pulmonary diseases induced by ambient ultrafine and engineered nanoparticles in twenty-first century, Natl. Sci. Rev. 3 (2016) 416-429.
150. Xie, G.; et al. On-demand release of CO₂ from photothermal hydrogels for accelerating skin wound healing. Chemical Engineering Journal 2021, 403, 126353.
151. Xie, H., et al, 2015. Scientific and engineering progress in CO₂ mineralization using industrial waste and natural minerals. Eng. 1, 150-157.
152. Xie, X.; et al. Ag nanoparticles cluster with pH-triggered reassembly in targeting antimicrobial applications. Advanced Functional Materials 2020, 30 (17), 2000511.
153. Xu, C.; et al. Preparation, characterization, and photocatalytic properties of silver carbonate. Applied surface science 2011, 257 (20), 8732-8736.
154. Yaashikaa, P.; et al. A review on photochemical, biochemical and electrochemical transformation of CO₂ into value-added products. Journal of CO₂ Utilization 2019, 33, 131-147.
155. Yan, J.; et al. Chemical Synthesis of Innovative Silver Nanohybrids with Synergistically Improved Antimicrobial Properties. International Journal of Nanomedicine 2023, 2295-2305.
156. Yan, T., et al, 2024. Mechanism and anti-corrosion measures of carbon dioxide corrosion in CCUS: a review. Iscience.
157. Zhang, S.; et al. An optimization model for carbon capture utilization and storage supply chain: A case study in Northeastern China. Appl. Energy 2018, 231, 194-206.
158. Zheng, L., et al, 2016. Corrosion mitigation via a pH stabilization method in monoethanolamine-based solutions for post-combustion CO₂ capture. Corros. Sci. 106, 281-292.
159. Zhou, Z. et al., Lung inflammation perturbation by engineered nanoparticles, Front. Bioeng. Biotechnol. 11 (2023) 1199230.
160. Zoneff, E., et al. Controlled oxygen delivery to power tissue regeneration. Nature Communications 2024, 15 (1), 4361.

What is claimed is:

1. A method for producing a metal carbonate nanoparticle, the method comprising:
(a) bubbling a gas comprising carbon dioxide through an aqueous solution comprising an amino acid and a base to create a precursor solution; and
(b) admixing a metal salt into the precursor solution to form a nanoparticle solution;
wherein the metal carbonate nanoparticle precipitates from the nanoparticle solution following step (b).

2. The method of claim 1, wherein the gas comprises concentrated CO₂ or a gas mixture comprising CO₂.

3. The method of claim 2, wherein the gas mixture comprising CO₂ comprises real flue gas (RFG), simulated flue gas (SFG), air, or any combination thereof.

4. The method of claim 1, wherein the amino acid comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof.

5. The method of claim 1, wherein the base comprises sodium hydroxide (NaOH), potassium hydroxide (KOH), barium hydroxide (Ba(OH)₂), lithium hydroxide (LiOH), Mg(OH)₂, Mn(OH)₂, Fe(OH)₂, Al(OH)₃, NH₄OH, K₂CO₃, KHCO₃, Na₂CO₃, NaHCO₃, or any combination thereof.

6. The method of claim 1, wherein the metal salt comprises CaCl₂), BaCl₂, SrCl₂, MgCl₂, AgNO₃, Ca(NO₃)₂, Ba(NO₃)₂, Ag₂SO₄, CaO, BaO, SrO, MgO, Ag₂O, ZnO, Fe₂O₃, CuO, CaCl₂), Ca(NO₃)₂, Ca(CH₃COO)₂, CaBr₂, or any combination thereof.

7. The method of claim 1, wherein the metal carbonate nanoparticles comprise CaCO₃, BaCO₃, SrCO₃, MgCO₃, Ag₂CO₃, ZnCO₃, FeCO₃, CuCO₃, PbCO₃, CdCO₃, or any combination thereof.

8. The method of claim 1, wherein the metal carbonate nanoparticles have a diameter of from about 10 nm to about 150 nm.

9. The method of claim 1, wherein the metal carbonate nanoparticles have a spherical morphology, a rod-like morphology, a fiber morphology, or a tube morphology.

10. The method of claim 9, wherein the metal carbonate nanoparticles have a rod-like morphology and a diameter-to-length ratio of from about 1:1 to about 1:6.

11. The method of claim 1, further comprising removing the metal carbonate nanoparticles from the nanoparticle solution.

12. The method of claim 11, wherein the metal carbonate nanoparticles are removed by centrifugation, decanting, or filtration and wherein a supernatant or filtrate comprising the amino acid is retained.

13. A metal carbonate nanoparticle produced by the method of claim 1.

14. An article or composition comprising the metal carbonate nanoparticle of claim 13.

15. The article or composition of claim 14, wherein the article or composition comprises a filler, a catalyst, a drug, an electronic component, a ceramic material, a wound care product, an engineered tissue, a surface coating, or any combination thereof.

16. The article or composition of claim 15, wherein the drug is an osteoporosis drug and wherein the metal carbonate nanoparticles comprise SrCO₃.

17. The article or composition of claim 15, wherein the metal carbonate nanoparticles comprise Ag₂CO₃ and the wound care product comprises a bandage, a gauze, a film, a foam, a hydrogel, or any combination thereof.

18. The article or composition of claim 15, wherein the metal carbonate nanoparticles comprise CaCO₃ and the filler comprises a plastic, cement, rubber, paint, or paper filler.

19. The article or composition of claim 15, wherein the metal carbonate nanoparticles comprise BaCO₃ and the ceramic material comprises a high dielectric constant ceramic material.

20. The article or composition of claim 15, wherein the metal carbonate nanoparticles comprise BaCO₃ and the electronic component comprises a capacitor or an inductor.
